SEQUENCE LISTING (2 sequences)

SEQ ID NO: 1 - protein, 20 aa, synthetic construct
  MOD_RES 1: note = Biotinylated
CKKKENALLRYLLDKDDTKD

SEQ ID NO: 2 - protein, 20 aa, synthetic construct
CKKKENALLRYLLDKDDTKD

(19) United States
(12) Patent Application Publication
(10) Pub. No.: US 2026/0083751 A1
(43) Pub. Date: Mar. 26, 2026

(54) ALLOSTERIC MODULATORS OF ANDROGEN RECEPTOR COACTIVATOR RECRUITMENT FOR CRPC THERAPY

(71) Applicant: University of Pittsburgh – Of the Commonwealth System of Higher Education, Pittsburgh, PA (US)

(72) Inventors: Paul A. Johnston, Pittsburgh, PA (US); Andrew P. Hinck, Pittsburgh, PA (US); Carlos J. Camacho, Pittsburgh, PA (US); Peter Wipf, Pittsburgh, PA (US); Zhou Wang, Pittsburgh, PA (US); Lee A. McDermott, Pittsburgh, PA (US)

(73) Assignee: University of Pittsburgh – Of the Commonwealth System of Higher Education, Pittsburgh, PA (US)

(21) Appl. No.: 19/108,095

(22) PCT Filed: Aug. 30, 2023
(86) PCT No.: PCT/US2023/073186

§ 371 Date: Feb. 28, 2025

Related U.S. Application Data
(60) Provisional application No. 63/402,819, filed on Aug. 31, 2022.

(51) Int. Cl. A61K 31/553; A61K 31/4045; A61K 31/4155; A61K 31/4439; A61K 31/454; A61K 31/497; A61K 31/501; A61P 35/00

(52) U.S. Cl. CPC A61K 31/553; A61K 31/4045; A61K 31/4155; A61K 31/4439; A61K 31/454; A61K 31/497; A61K 31/501; A61P 35/00

(57) ABSTRACT

Small molecule allosteric modulators of androgen receptor are disclosed. The compounds may inhibit androgen receptor coactivator recruitment, such as by preventing formation of protein-protein interaction (PPI) complexes and/or disrupting PPI complexes. A method of treating prostate cancer, such as castration-resistant prostate cancer, includes administering a small molecule allosteric modulator of androgen receptor.

### CROSS-REFERENCE TO RELATED APPLICATION

This application claims the benefit of the earlier filing date of U.S. Provisional Application No. 63/402,819, filed Aug. 31, 2022, which is incorporated by reference in its entirety herein.

### ACKNOWLEDGMENT OF GOVERNMENT SUPPORT

This invention was made with government support under CA183882, CA160423, and NS073889 awarded by the National Institutes of Health. The government has certain rights in the invention.

### FIELD

Small molecule allosteric modulators of androgen receptor activity are disclosed, as well as methods of making and using the compounds.

### INCORPORATION OF ELECTRONIC SEQUENCE LISTING

The electronic sequence listing, submitted herewith as an XML file named 8123-108659-02.xml (3,083 bytes), created on Aug. 28, 2023, is herein incorporated by reference in its entirety.

### BACKGROUND

Prostate cancer (PC) is the 2nd leading cause of cancer death among men in the United States, 12.5% of whom will be diagnosed with PC in their lifetimes. In 2022, it is estimated there will be >265,000 new PC cases and 34,500 related deaths in the USA alone. Although 5-year relative survival rates for men with local or regional PC are ≥99%, rates decline to 31% for distant metastatic disease with a median survival of 36 months. Despite increased PC therapy options, most metastatic castration resistant PC (mCRPC) patients develop drug resistance and overall survival is extended by only 3-5 months. mCRPC remains incurable and is a major unmet clinical need. Despite castrate testosterone levels, mCRPC tumors retain a dependence on the androgen receptor (AR) that contributes to drug resistance mechanisms. Ligand bound AR activates target gene transcription after DNA binding by recruiting and forming protein-protein interaction (PPI) complexes with coactivators.

### SUMMARY

This disclosure concerns compounds that are small molecule allosteric modulators of androgen receptor (AR) activity, as well as methods of making and using the compounds. In some aspects, the compound is a hydrobenzo-oxazepine, a thiadiazol-5-piperidine carboxamide, a fluorophenyl-methyl-indole, a phenyl-methyl-indole, a heteroaliphatic- or heteroaryl-substituted methyl-indole, or any combination thereof.

A method of modulating AR-mediated activity includes contacting AR with an effective amount of a compound as disclosed herein. Contacting AR with the effective amount of the compound inhibits AR-coactivator (AR-CoA) protein-protein interaction (PPI) complexes, inhibits prostate specific antigen (PSA) expression in prostate epithelial cells and/or prostate cancer cells, inhibits PSA secretion by prostate epithelial cells and/or prostate cancer cells, inhibits AR-mediated PSA promoter-driven transcription in prostate cancer cells, inhibits AR splice variant 7 (AR-V7)-mediated PSA promoter driven transcription in prostate cancer cells, inhibits ubiquitin conjugating enzyme E2 C (UBE2C) promoter-driven transcription in prostate cancer cells, inhibits growth of prostate cancer cells, or any combination thereof.

In some aspects, inhibiting AR-CoA PPI complexes comprises reducing formation of and/or disrupting formed AR-CoA PPI complexes. In some examples, the CoA comprises transcriptional intermediary factor 2 (TIF2), steroid receptor coactivator (SRC1), or a combination thereof.

In any of the foregoing or following aspects, contacting may be performed in vivo, such as by administering the effective amount of the compound to a subject. In some aspects, treating prostate cancer comprises administering a therapeutically effective amount of a compound as disclosed herein to a subject. The subject may have, or be suspected of having, prostate cancer. In some implementations, the prostate cancer is CRPC, such as mCRPC.

The foregoing and other objects, features, and advantages of the invention will become more apparent from the following detailed description, which proceeds with reference to the accompanying figures.

### BRIEF DESCRIPTION OF THE DRAWINGS

FIG. 1 is a table showing activities of hydrobenzo-oxazepines in several assays.

FIG. 2 is a bar graph showing results of an androgen receptor cellular thermal shift assay (AR-CETSA) of the hydrobenzo-oxazepines of FIG. 1.

FIG. 3 is a table showing activities of thiadiazol-5-piperidine carboxamides in several assays.

FIGS. 4A-4C show that an exemplary thiadiazol-5-piperidine carboxamide inhibited DHT-induced AR stability at 46° C.; FIGS. 4A-4B are bar graphs showing activity of the compound (FIG. 4A) and controls (FIG. 4B) in an AR-CETSA assay; FIG. 4C is a graph showing inhibition of DHT-induced AR thermal stabilization as a function of compound concentration.

FIGS. 5A and 5B are a bar graph (FIG. 5A) and graph (FIG. 5B) showing that 20 μM and 50 μM concentrations of the compound of FIGS. 4A-4C inhibited DHT-enhanced AR stability at 46° C.

FIG. 6 is a table showing activities of methyl indoles in several assays.

FIGS. 7A-7D show results of AR CETSA assays with the methyl indoles. FIG. 7A shows that exposure of C4-2 cells to methyl indole compounds enhanced the AR signal to levels produced by cells exposed to DHT; FIG. 7B shows that exposures of cells AR antagonists and methyl indole compounds did not enhance AR thermal stability at 46° C.; FIG. 7C shows the controls; FIG. 7D shows that most of the methyl indoles inhibited ability of DHT to enhance AR thermal stability in a concentration-dependent manner.

FIGS. 8A-8C are a bar graph (FIG. 8A) and graphs (FIGS. 8B, 8C) showing that 20 μM and 50 μM concentrations of two methyl indoles inhibited DHT-enhanced AR stability at 46° C.

FIGS. 9A-9L show bioactivity profiles of three representative compounds-inhibition of DHT-induced AR-TIF2 PPI formation (FIG. 9A), disruption of pre-formed DHT-induced AR-TIF2 PPI complexes (FIG. 9B), inhibition of DHT-induced AR-TIF2 mammalian 2-hybrid PPI formation (FIG. 9C), inhibition of DHT-induced AR-SRC-1 mammalian 2-hybrid PPI formation (FIG. 9D), inhibition of DHT-induced AR-TIF2 box 3 LXXLL peptide binding (FIG. 9E), inhibition of H3-DHT binding to recombinant AR-LBD (FIG. 9F), inhibition of DHT-induced PSA6.1-luciferase reporter activity in C4-2 CRPC cells (FIG. 9G), inhibition of constitutive PSA6.1-luciferase reporter activity in AR-V7-GFP-PC-3 cells (FIG. 9H), inhibition of constitutive UBE2C-luciferase reporter activity in AR-V7-GFP-PC-3 cells (FIGS. 9I), and S1-1 (FIG. 9J), S2-6 (FIG. 9K), and S3-11 (FIG. 9L) growth inhibition in PC cell lines positive (LNCaP, C4-2, & 22Rv1) or negative (PC-3 & DU-145) for AR. Representative normalized % inhibition curves from one of three independent experiments that were conducted in 10-point concentration response assays performed in triplicate (n=3) wells for each compound concentration are presented for S1-1, S2-6, and S3-11. Symbols and error bars represent the mean±sd (n=3) normalized % inhibition at each compound concentration. The mean±SD IC₅₀s for S1-1, S2-6, and S3-11 in each of the bioassays are presented in FIGS. 1, 3, and 6, respectively.

FIGS. 10A-10C show that enzalutamide inhibits DHT-enhanced PSA expression in C4-2 cells. FIG. 10A is a western blot showing relative PSA and β-actin expression levels; FIG. 10B shows quantification of the PSA western blot results by scanning densitometry; FIG. 10C shows quantification the β-actin western blot results by scanning densitometry. Representative data from three independent experiments are presented.

FIGS. 11A-11D show inhibition of AR regulated prostate specific antigen (PSA) biomarker expression and secretion in C4-2 castration resistant prostate cancer cells by compounds S1-1, S2-6, and S3-11. FIG. 11A is a western blot showing relative PSA expression levels in C4-2 cells±DHT; PSA expression levels in C4-2 cells cultured for 24 h±10 nM DHT were compared by SDS-PAGE and western blots that were probed with a specific anti-PSA antibody. FIG. 11B is a graph showing quantification of the PSA western blots by scanning densitometry. FIG. 11C show relative PSA secretion levels in C4-2 conditioned media±DHT; relative PSA secretion levels in conditioned media collected from the corresponding C4-2 monolayers cultured for 24 h±10 nM DHT were compared on dot blots that were probed with the same PSA antibody. FIG. 11D shows quantification of PSA dot blots by scanning densitometry. Representative data from three independent experiments are presented.

FIGS. 12A-12D show inhibition of DHT-enhanced AR thermal stability in western blots of C4-2 castration resistant prostate cancer cells by the S1-1, S2-6, and S3-11 representative hits. FIG. 12A shows the amount of soluble AR protein in heat shocked C4-2 cell lysates; the amount of soluble AR protein remaining in heat shocked C4-2 cell lysis supernatants after centrifugation were compared by SDS-PAGE and western blots that were probed with a specific anti-AR antibody. FIG. 12B shows quantification of soluble AR levels on western blots of heat shocked C4-2 cell lysates by scanning densitometry; AR exhibited a characteristic reduction in soluble protein at increasing temperatures with a 50% reduction T_{agg} value of 44.9° C. using the left Y axis; for comparison the amount of total soluble protein determined in the BCA assay of cell lysate supernatants of C4-2 cells that were heat shocked at the indicated temperatures are presented using the right Y axis. FIG. 12C shows effects of S1-1, S2-6, or S3-11 pretreatment of C4-2 cells on AR thermal stability. FIG. 12D shows quantification of soluble AR levels on western blots of compound treated heat shocked C4-2 cell lysates by scanning densitometry. Representative data from three independent experiments are presented.

FIGS. 13A-13D show that S1-1, S2-6, and S3-11 do not enhance TIF2 thermal stability in western blots of C4-2 castration resistant prostate cancer cells. FIG. 13A shows the amount of soluble TIF2 protein in heat shocked C4-2 cell lysates. FIG. 13B shows quantification of soluble TIF2 protein in heat shocked C4-2 cell lysates by scanning densitometry; TIF2 exhibited a characteristic reduction in soluble protein at increasing temperatures with a 50% reduction T_{agg} value of 43.6° C. using the left Y axis; for comparison the amount of total soluble protein determined in the BCA assay of cell lysate supernatants of C4-2 cells that were heat shocked at the indicated temperatures are presented using the right Y axis. FIG. 13C shows the effects of S1-1, S2-6, or S3-11 pretreatment of C4-2 cells on TIF2 thermal stability. FIG. 13D shows quantification of soluble TIF2 levels on western blots of compound treated heat shocked C4-2 cell lysates by scanning densitometry. Representative data from three independent experiments are presented.

FIGS. 14A-14C show that enzalutamide inhibits DHT-enhanced AR thermal stability. FIG. 14A shows effects of enzalutamide pretreatment of C4-2 cells on AR thermal stability. FIG. 14B shows quantification of soluble AR levels on western blots of compound treated heat shocked C4-2 cell lysates by scanning densitometry. FIG. 14C shows AlphaScreen® AR CETSA (BMG Labtech, Cary, NC)—effects of enzalutamide pretreatment on AR thermal stability; AR AlphaScreen® RLU signals for lysates from non-heat shocked C4-2 cells (left, black), C4-2 cells heat shocked at 46° C. for 5 min (middle, white), and C4-2 cells pre-treated with 10 nM DHT for 1 h before heat shocking at 46° C. for 5 min (right, gray) are presented. The bars (FIG. 14C) and error bars represent the mean±SD (n=3) of triplicate determinations. Representative data from one of three independent experiments are presented.

FIGS. 15A-15D show AlphaScreen® CETSA format inhibition of DHT-enhanced AR thermal stability in C4-2 castration resistant prostate cancer cells by S1-1, S2-6, and S3-11. FIG. 15A shows AR CETSA plate controls; AR RLU signals in the absence of beads, antibodies, or cell lysates are compared to the signals for lysates from non-heat shocked C4-2 cells, C4-2 cells heat shocked at 46° C. for 5 min, and C4-2 cells pre-treated with 10 nM DHT for 1 h before heat shocking at 46° C. for 5 min. FIG. 15B shows effects of S1-1, S2-6 or S3-11 pretreatment on AR thermal stability; AR RLU signals for lysates from non-heat shocked C4-2 cells (left, black), C4-2 cells heat shocked at 46° C. for 5 min (middle, white), and C4-2 cells pre-treated with 10 nM DHT for 1 h before heat shocking at 46° C. for 5 min (right) are presented. FIG. 15C shows effects of S2-6 on the isothermal concentration fingerprint of DHT; C4-2 cells were pretreated for 1 h with DMSO or either 20 μM or 50 μM S2-6 prior to DHT treatment and heat shock. FIG. 15D shows effects of S3-11 on the isothermal concentration fingerprint of DHT; C4-2 cells were pretreated for 1 h with DMSO or either 20 μM or 50 μM S3-11 prior to DHT treatment and heat shock.

### DETAILED DESCRIPTION

Androgen ablation/depravation therapy (AAT/ADT) targets the earliest points of androgen receptor (AR) signaling, either the production or action of testicular androgens that provide critical growth and survival signals to prostate. Despite increased prostate cancer (PC) therapy options, most metastatic castration resistant PC (mCRPC) patients develop drug resistance and overall survival is extended by only 3-5 months. AAT toxicities and adverse events (AEs) include muscle atrophy, anemia, cognitive dysfunction, and treatment induced bone loss, and newer PC drugs share these liabilities.

Ligand bound AR activates target gene transcription after DNA binding by recruiting and forming protein-protein interaction (PPI) complexes with coactivators like Transcription Intermediary Factor 2 (TIF2/SRC-2). Allosteric modulator (AM) drugs that bind to AR to block the recruitment of coactivators for transcriptional activation would be novel. AM binding pockets are generally structurally, conformationally and functionally different than endogenous orthosteric ligand (OSL) binding sites and AM drugs can offer distinct advantages. AMs exhibit superior target selectivity than OSLs because their binding sites are less conserved and therefore reduce the incidence of side effects (SE) and/or adverse events (AE). Since AMs do not compete with endogenous OSLs, effective drug concentrations may be lower, further reducing potential SEs and AEs. AMs have no agonist activity and only exert functional effects when OSLs are present, protecting the spatiotemporal effects of the natural ligand. Some AMs also may be more chemically tractable with better physiochemical properties than OSLs.

This disclosure concerns aspects of allosteric modulators that bind to AR. In some aspects, the compound inhibits AR coactivator recruitment, e.g., by preventing formation of PPI complexes and/or disrupting PPI complexes. In some implementations, the compounds may additionally, or alternatively, inhibit prostate specific antigen (PSA) expression in prostate epithelial cells and/or prostate cancer cells, inhibit PSA secretion by prostate epithelial cells and/or prostate cancer cells, inhibit AR-mediated PSA promoter-driven transcription in prostate cancer cells, inhibit AR splice variant (AR-V7)-mediated PSA promoter driven transcription in prostate cancer cells, inhibit ubiquitin conjugating enzyme E2 C (UBE2C) promoter-driven transcription in prostate cancer cells, inhibit growth of prostate cancer cells, or any combination thereof. Some of the disclosed compounds may be useful for treating prostate cancer, such as mCRPC.

### I. Definitions and Abbreviations

The following explanations of terms and abbreviations are provided to better describe the present disclosure and to guide those of ordinary skill in the art in the practice of the present disclosure. As used herein, “comprising” means “including.”

Unless explained otherwise, all technical and scientific terms used herein have the same meaning as commonly understood to one of ordinary skill in the art to which this disclosure belongs. Although methods and materials similar or equivalent to those described herein can be used in the practice or testing of the present disclosure, suitable methods and materials are described below. The materials, methods, and examples are illustrative only and not intended to be limiting. Other features of the disclosure are apparent from the following detailed description and the claims.

The disclosure of numerical ranges should be understood as referring to each discrete point within the range, inclusive of endpoints, unless otherwise noted. Unless otherwise indicated, all numbers expressing quantities of components, molecular weights, percentages, temperatures, times, and so forth, as used in the specification or claims are to be understood as being modified by the term “about,” where the term “about,” unless otherwise specified, means±5% relative to the stated value(s). Accordingly, unless otherwise implicitly or explicitly indicated, or unless the context is properly understood by a person of ordinary skill in the art to have a more definitive construction, the numerical parameters set forth are approximations that may depend on the desired properties sought and/or limits of detection under standard test conditions/methods as known to those of ordinary skill in the art. When directly and explicitly distinguishing aspects from discussed prior art, the aspect numbers are not approximates unless the word “about” is recited.

Although there are alternatives for various components, parameters, operating conditions, etc. set forth herein, that does not mean that those alternatives are necessarily equivalent and/or perform equally well. Nor does it mean that the alternatives are listed in a preferred order unless stated otherwise.

Protected derivatives of the disclosed compounds also are contemplated. A variety of suitable protecting groups for use with the disclosed compounds are disclosed in Wuts et al., Greene's Protective Groups in Organic Synthesis; 3rd Ed.; John Wiley & Sons, New York, 2007. In general, protecting groups are removed under conditions which will not affect the remaining portion of the molecule. These methods are well known in the art and include acid hydrolysis, hydrogenolysis and the like. One preferred method involves the removal of an ester, such as cleavage of a phosphonate ester using Lewis acidic conditions, such as in TMS-Br mediated ester cleavage to yield the free phosphonate. A second preferred method involves removal of a protecting group, such as removal of a benzyl group by hydrogenolysis utilizing palladium on carbon in a suitable solvent system such as an alcohol, acetic acid, and the like or mixtures thereof. A t-butoxy-based group, including t-butoxy carbonyl protecting groups can be removed utilizing an inorganic or organic acid, such as HCl or trifluoroacetic acid, in a suitable solvent system, such as water, dioxane and/or methylene chloride. Another exemplary protecting group, suitable for protecting amino and hydroxy functions amino is trityl. Other conventional protecting groups are known and suitable protecting groups can be selected by those of skill in the art in consultation with Greene and Wuts, Protective Groups in Organic Synthesis; 3rd Ed.; John Wiley & Sons, New York, 1999. When an amine is deprotected, the resulting salt can readily be neutralized to yield the free amine. Similarly, when an acid moiety, such as a phosphonic acid moiety is unveiled, the compound may be isolated as the acid compound or as a salt thereof.

Particular examples of the presently disclosed compounds may include one or more asymmetric centers; thus these compounds can exist in different stereoisomeric forms. Accordingly, compounds and compositions may be provided as individual pure enantiomers or as stereoisomeric mixtures, including racemic mixtures. In certain aspects the compounds disclosed herein are synthesized in or are purified to be in substantially enantiopure form, such as in a 90% enantiomeric excess, a 95% enantiomeric excess, a 97% enantiomeric excess or even in greater than a 99% enantiomeric excess, such as in enantiopure form.

Definitions of common terms in chemistry may be found in Richard J. Lewis, Sr. (ed.), Hawley's Condensed Chemical Dictionary, published by John Wiley & Sons, Inc., 2016 (ISBN 978-1-118-13515-0).

Administration/administering: “Administration of” and “administering a” compound should be understood to mean providing a compound, a prodrug of a compound, or a pharmaceutical composition as described herein. The compound or composition can be administered by another person to the subject (e.g., intravenously) or it can be self-administered by the subject (e.g., tablets). Co-administration or co-administering means administering two or more therapeutic agents or modalities. Co-administration may occur simultaneously or sequentially in any order, and may occur by the same or different routes of administration. When administering simultaneously, the two or more therapeutic agents may be present in a single pharmaceutical composition or in separate pharmaceutical compositions. When administered sequentially, the two or more therapeutic agents are administered such that the therapeutic time windows of the agents coincide or overlap.
ADT: androgen deprivation therapies
AE: adverse events
AF-1: activation function 1 surface
AF-2: activation function 2 surface
Aliphatic: A substantially hydrocarbon-based compound, or a radical thereof (e.g., C₆H₁₃, for a hexane radical), including alkanes, alkenes, alkynes, and further including straight- and branched-chain arrangements, and all stereo and position isomers as well. Cyclic aliphatic compounds or radicals may be referred to as cycloaliphatic. Unless expressly stated otherwise, an aliphatic group contains from one to twenty-five carbon atoms; for example, from one to fifteen, from one to ten, from one to six, or from one to four carbon atoms. The term “lower aliphatic” refers to an aliphatic group containing from one to ten carbon atoms. An aliphatic chain may be substituted or unsubstituted. Unless expressly referred to as an “unsubstituted aliphatic,” an aliphatic group can either be unsubstituted or substituted. A substituted aliphatic compound includes at least one sp³-hybridized carbon or two sp²-hybridized carbons bonded with a double bond or at least two sp-hybridized carbons bonded with a triple bond. An aliphatic group can be substituted with one or more substituents (up to two substituents for each methylene carbon in an aliphatic chain, or up to one substituent for each carbon of a —C═C— double bond in an aliphatic chain, or up to one substituent for a carbon of a terminal methine group). Exemplary substituents include, but are not limited to, alkyl, alkenyl, alkynyl, alkoxy, alkylamino, alkylthio, acyl, aldehyde, amide, amino, aminoalkyl, aryl, arylalkyl, carboxyl, cyano, cycloalkyl, dialkylamino, halo, haloaliphatic, heteroaliphatic, heteroaryl, heterocycloaliphatic, hydroxyl, oxo, sulfonamide, sulfhydryl, thioalkoxy, or other functionality.
AM: allosteric modulator
AR: androgen receptor
AR-LBD: androgen receptor ligand binding domain
AR-V7: androgen receptor splice variant 7
Aryl: A monovalent aromatic carbocyclic group of, unless specified otherwise, from 6 to 15 carbon atoms having a single ring (e.g., phenyl) or multiple condensed rings in which at least one ring is aromatic (e.g., indole, benzodioxole, and the like), provided that the point of attachment is through an atom of an aromatic portion of the aryl group and the aromatic portion at the point of attachment contains only carbons in the aromatic ring. If any aromatic ring portion contains a heteroatom, the group is a heteroaryl and not an aryl. Aryl groups are monocyclic, bicyclic, tricyclic or tetracyclic.
BCA: bicinchoninic acid
BP-1: binding pocket 1 adjacent to AR orthosteric ligand binding site
BF-3: binding function 3 allosteric pocket of AR
BSA: bovine serum albumin
CETSA: Cellular enhanced thermal stability assays
CoA: coactivator
CoR: corepressor
CRPC: castration-resistant prostate cancer; mCRPC is metastatic CRPC.
DBD: DNA-binding domain
DHT: dihydrotestosterone
DMSO: dimethyl sulfoxide
Effective amount: An amount of the compound or composition sufficient to achieve a particular desired result, such as to modulate activity of a receptor; to elicit a desired biological or medical response in a tissue, system, subject or patient; to treat a specified disorder or disease; to ameliorate or eradicate one or more of its symptoms; and/or to prevent the occurrence of the disease or disorder. The amount of a compound which constitutes an “effective amount” may vary depending on the compound, the desired result, the disease state and its severity, the age of the patient to be treated, and the like.
ER: estrogen nuclear receptor
GR: glucocorticoid nuclear receptor
HCS: high-content screening
Heterocycle/Heterocyclic: Refers to a closed-ring compound, or radical thereof as a substituent bonded to another group, particularly other organic groups, where at least one atom in the ring structure is other than carbon, and typically is oxygen, sulfur and/or nitrogen. A heterocycle may be aryl (heteroaryl) or aliphatic (heterocycloaliphatic).
HTS: high throughput screening
IC₅₀: 50% inhibition concentration
LBD: ligand-binding domain
LUC: luciferase
mCRPC: metastatic castrate-resistant prostate cancer
mCSPC: metastatic hormone/castrate sensitive prostate cancer
MOA: mechanism of action
MR: mineralocorticoid nuclear receptor
NR: nuclear receptor
NTD: amino-terminal domain
OSL: orthosteric ligand
p160/SRC: p160 steroid receptor coactivator
PARPi: poly adenosine-5′-diphosphate ribose polymerase inhibitors
PC: prostate cancer
PPI: protein-protein interaction
PPIB: protein-protein interaction biosensor
PR: progesterone nuclear receptor
PSA: prostate specific antigen
RLUs: relative light units
Subject: An animal (human or non-human) subjected to a treatment, observation or experiment. Includes both human and veterinary subjects, including human and non-human mammals, such as rats, mice, cats, dogs, pigs, horses, cows, and non-human primates.
Substituent: An atom or group of atoms that replaces another atom in a molecule as the result of a reaction. The term “substituent” typically refers to an atom or group of atoms that replaces a hydrogen atom, or two hydrogen atoms if the substituent is attached via a double bond, on a parent hydrocarbon chain or ring. The term “substituent” may also cover groups of atoms having multiple points of attachment to the molecule, e.g., the substituent replaces two or more hydrogen atoms on a parent hydrocarbon chain or ring. In such instances, the substituent, unless otherwise specified, may be attached in any spatial orientation to the parent hydrocarbon chain or ring. Exemplary substituents include, for instance, alkyl, alkenyl, alkynyl, alkoxy, alkylamino, alkylthio, acyl, aldehyde, amido, amino, aminoalkyl, aryl, arylalkyl, arylamino, carbonate, carboxyl, cyano, cycloalkyl, dialkylamino, halo, haloaliphatic (e.g., haloalkyl), haloalkoxy, heteroaliphatic, heteroaryl, heterocycloaliphatic, hydroxyl, oxo, sulfonamide, sulfhydryl, thio, and thioalkoxy groups.
Substituted: A fundamental compound, such as an aryl or aliphatic compound, or a radical thereof, having coupled thereto one or more substituents, each substituent typically replacing a hydrogen atom on the fundamental compound. A person of ordinary skill in the art will recognize that compounds disclosed herein may be described with reference to particular structures and substituents coupled to such structures, and that such structures and/or substituents also can be further substituted, unless expressly stated otherwise or context dictates otherwise. Solely by way of example and without limitation, a substituted aryl compound may have an aliphatic group coupled to the closed ring of the aryl base, such as with toluene. Again solely by way of example and without limitation, a long-chain hydrocarbon may have a hydroxyl group bonded thereto. Groups which are substituted (e.g, substituted alkyl), may in some aspects be substituted with a group which is substituted (e.g, substituted aryl). In some aspects, the number of substituted groups linked together is limited to two (e.g, substituted alkyl is substituted with substituted aryl, wherein the substituent present on the aryl is not further substituted). In some aspects, a substituted group is not substituted with another substituted group (e.g, substituted alkyl is substituted with unsubstituted aryl).
TA: transcriptional activation
TAU1: transcription activation unit 1
TAU2: transcription activation unit 2
Therapeutically effective amount or dose: An amount sufficient to provide a beneficial, or therapeutic, effect to a subject or a given percentage of subjects. Ideally, a therapeutically effective amount of an agent is an amount sufficient to inhibit or treat the disease without causing a substantial cytotoxic effect in the subject.
Therapeutic time window: The length of time during which an effective, or therapeutic dose, of a compound remains therapeutically effective in vivo.
TIF2: Transcriptional Intermediary Factor 2
Treating or treatment: With respect to disease, either term includes (1) preventing the disease, e.g., causing the clinical symptoms of the disease not to develop in an animal that may be exposed to or predisposed to the disease but does not yet experience or display symptoms of the disease, (2) inhibiting the disease, e.g., arresting the development of the disease or its clinical symptoms, or (3) relieving the disease, e.g., causing regression of the disease or its clinical symptoms.

### II. Compounds

Disclosed herein are compounds for modulating AR-mediated activity. The compound may be an allosteric modulator that binds to AR. In some aspects, the compound is a hydrobenzo-oxazepine, a thiadiazol-5-piperidine carboxamide, a fluorophenyl-methyl-indole, a phenyl-methyl-indole, a heteroaliphatic- or heteroaryl-substituted methyl-indole, or any combination thereof. Some aspects of the disclosed compounds are useful for inhibiting or treating prostate cancer. In some aspects, AR is contacted with an effective amount of a compound disclosed herein, thereby inhibiting AR-coactivator (AR-CoA) protein-protein interaction (PPI) complexes, inhibiting prostate specific antigen (PSA) expression in prostate epithelial cells and/or prostate cancer cells, inhibiting PSA secretion by prostate epithelial cells and/or prostate cancer cells, inhibiting AR-mediated PSA promoter-driven transcription in prostate cancer cells, inhibiting androgen receptor splice variant 7 (AR-V7)-mediated PSA promoter driven transcription in prostate cancer cells, inhibiting ubiquitin conjugating enzyme E2 C (UBE2C) promoter-driven transcription in prostate cancer cells, inhibiting growth of prostate cancer cells, or any combination thereof.

Aspects of the disclosed hydrobenzo-oxazepines may have a general formula (I):

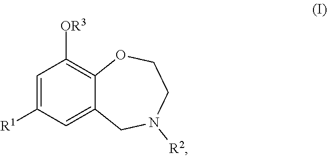

where R¹ is aryl or heteroaryl, R² is a heterocycle, and R³ is —H or alkyl. In some aspects, R³ is —H or C₁-C₅alkyl. In certain aspects, R³ is —H or —CH₃. In some implementations, R¹ is phenyl or thiophenyl and R² is thiazolyl or pyrimidinyl.

Aspects of the disclosed thiadiazol-5-piperidine carboxamides may have a general formula (II):

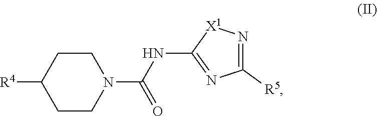

where X¹ is S, O, or N; R⁴ is —X²—R or halo, where X² is CH₂, S, or O, and R^{a} is aryl or heteroaryl; and R⁵ is aliphatic or a heterocycle. In some aspects, R⁵ is C₁-C₅ alkyl. In certain aspects, R⁵ is —CH₃ and X¹ is S. In some implementations, X² is S or C₁-C₅ alkyl. In certain implementations, X² is S or CH₂ and R^{a} is phenyl, imidazolyl, or pyrrolidinyl.

Aspects of the disclosed indoles may have a general formula III:

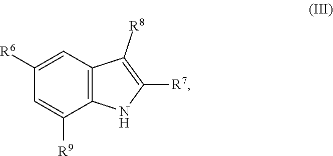

where R⁶ is —CH₂N(H)Y(CH₂)ₘ—R^{b}, H, halo, or alkyl, where Y is C(O) or S(O)₂, R is a heterocycle or —N(H)C(O)(CH₂)ₘCH₃, and each m independently is 0, 1, 2, or 3. R⁷ is is aryl, heteroaryl, H, or alkyl. R⁸ is alkyl, aryl, a heterocycle, or H. R⁹ is H, alkyl, or —CH₂N(H)Y(CH₂)ₘ—R^{b}. In any of the foregoing or following aspects, an alkyl group of R⁷-R⁹ may be C₁-C₅ alkyl. At least one of R⁶ and R⁹ is not H. At least one of R⁷ and R⁸ is not H. In some implementations, Y is C(O). In some aspects, R⁶ is —CH₂N(H)C(O)(CH₂)ₘ—R^{b}, H, F, or —CH₃, where m is 0, 1, or 2, and R^{b} is diazolyl, pyrimidinyl, pyridinyl,

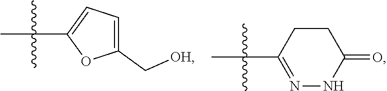 [SMILES: *c1ccc(CO)o1 ; *C1=NNC(=O)CC1]

or —N(H)C(O)CH₃. In certain aspects, R^{b} is

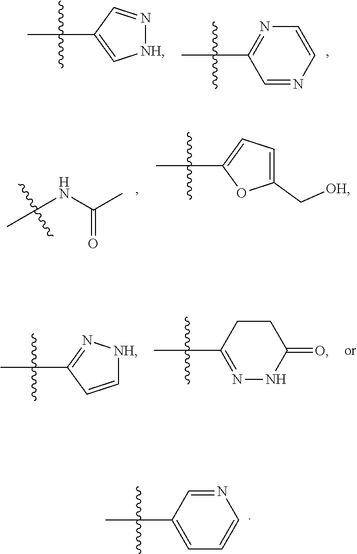

In some aspects, R⁷ is

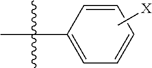

where X is halo, or R⁷ is H or —CH₂CH₃. In some examples, X is F. In some aspects, R⁸ is —CH₃,

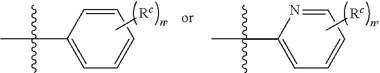

where n is 1 or 2, and each R^{c} independently is halo, —OR^{d}, —C(O)NHR^{d}, or aminoalkyl, where each R^{d} independently is H or C₁-C₅ alkyl.

In some aspects, one of R⁶-R⁹ is methyl, and the compound is a methyl-indole. In some implementations, the methyl-indole has a structure according to formula IIIA, IIIB, or IIIC:

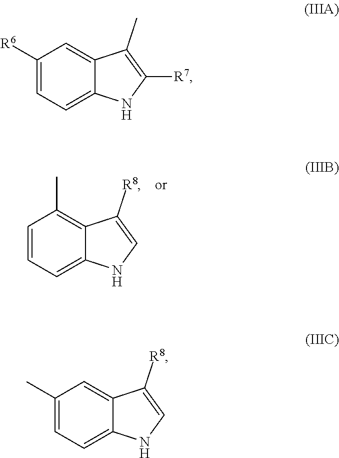

where R⁶ is —CH₂N(H)Y(CH₂)ₘ—R^{b}, Y is CO or S(O)₂; R^{b} is a heterocycle or —N(H)C(O)(CH₂)ₘCH₃; R⁷ is aryl or heteroaryl; and R⁸ is aryl or a heterocycle.

In some implementations, the compound has a structure according to formula IIIA, where R⁷ is fluorophenyl, H, or —CH₂CH₃, and R⁶ is

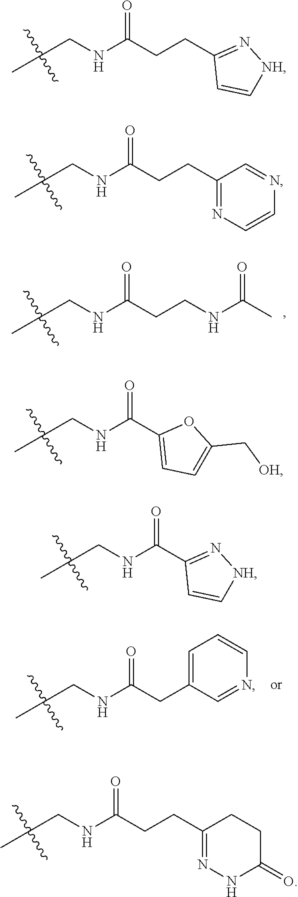

In some aspects, the compound has a structure according to formula IIIB or IIIC, where R⁸

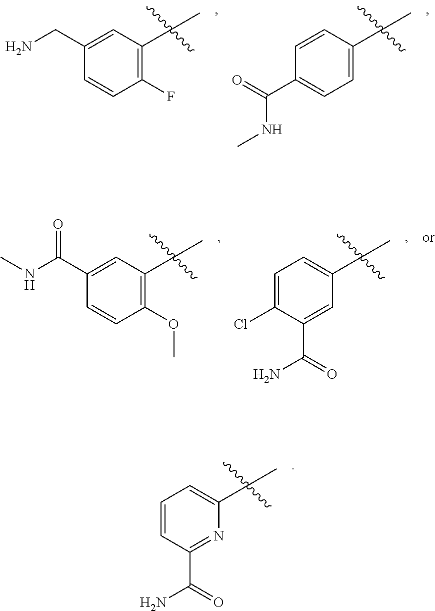

Exemplary compounds include, but are not limited to the compounds of Table A:

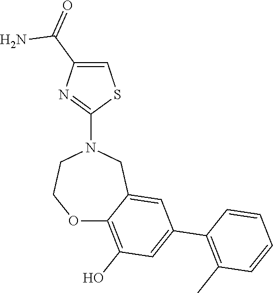 [SMILES: Cc1ccccc1-c1cc(O)c2c(c1)CN(c1nc(C(N)=O)cs1)CCO2]

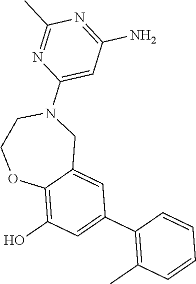 [SMILES: Cc1nc(N)cc(N2CCOc3c(O)cc(-c4ccccc4C)cc3C2)n1]

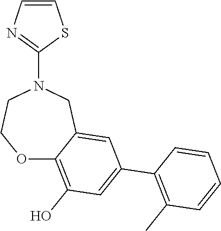 [SMILES: Cc1ccccc1-c1cc(O)c2c(c1)CN(c1nccs1)CCO2]

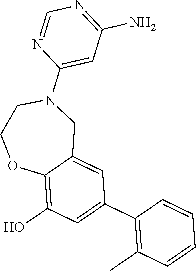 [SMILES: Cc1ccccc1-c1cc(O)c2c(c1)CN(c1cc(N)ncn1)CCO2]

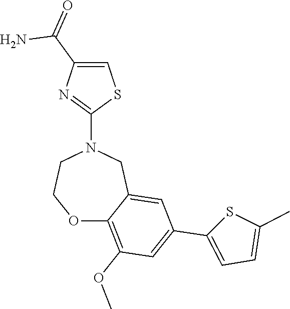 [SMILES: COc1cc(-c2ccc(C)s2)cc2c1OCCN(c1nc(C(N)=O)cs1)C2]

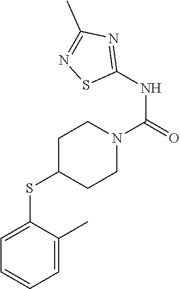 [SMILES: Cc1nsc(NC(=O)N2CCC(Sc3ccccc3C)CC2)n1]

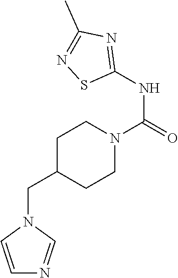 [SMILES: Cc1nsc(NC(=O)N2CCC(Cn3ccnc3)CC2)n1]

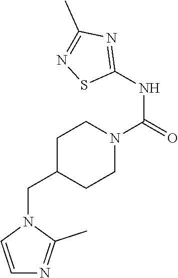 [SMILES: Cc1nsc(NC(=O)N2CCC(Cn3ccnc3C)CC2)n1]

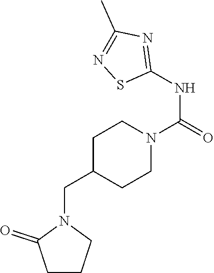 [SMILES: Cc1nsc(NC(=O)N2CCC(CN3CCCC3=O)CC2)n1]

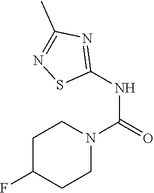 [SMILES: Cc1nsc(NC(=O)N2CCC(F)CC2)n1]

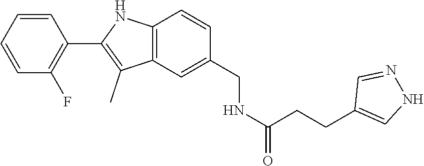 [SMILES: Cc1c(-c2ccccc2F)[nH]c2ccc(CNC(=O)CCc3cn[nH]c3)cc12]

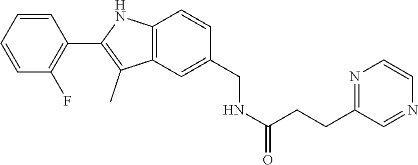 [SMILES: Cc1c(-c2ccccc2F)[nH]c2ccc(CNC(=O)CCc3cnccn3)cc12]

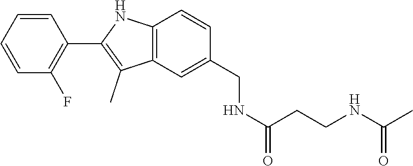 [SMILES: CC(=O)NCCC(=O)NCc1ccc2[nH]c(-c3ccccc3F)c(C)c2c1]

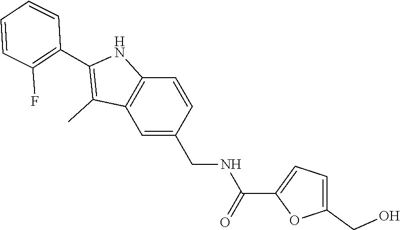 [SMILES: Cc1c(-c2ccccc2F)[nH]c2ccc(CNC(=O)c3ccc(CO)o3)cc12]

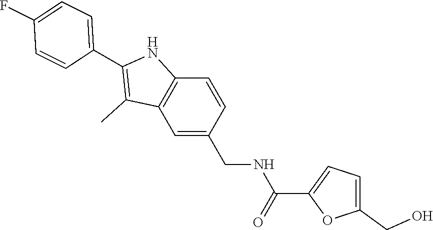 [SMILES: Cc1c(-c2ccc(F)cc2)[nH]c2ccc(CNC(=O)c3ccc(CO)o3)cc12]

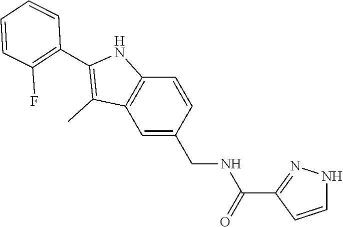 [SMILES: Cc1c(-c2ccccc2F)[nH]c2ccc(CNC(=O)c3cc[nH]n3)cc12]

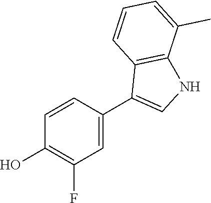 [SMILES: Cc1cccc2c(-c3ccc(O)c(F)c3)c[nH]c12]

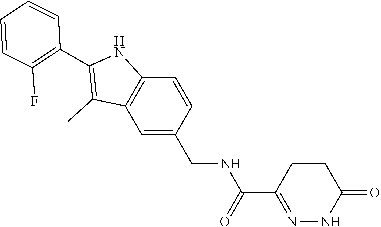 [SMILES: Cc1c(-c2ccccc2F)[nH]c2ccc(CNC(=O)C3=NNC(=O)CC3)cc12]

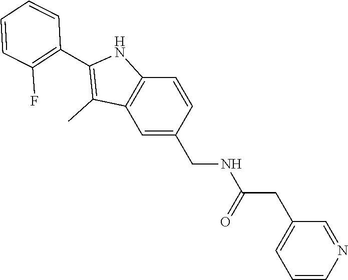 [SMILES: Cc1c(-c2ccccc2F)[nH]c2ccc(CNC(=O)Cc3cccnc3)cc12]

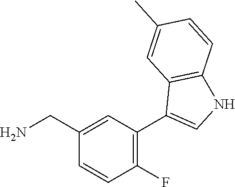 [SMILES: Cc1ccc2[nH]cc(-c3cc(CN)ccc3F)c2c1]

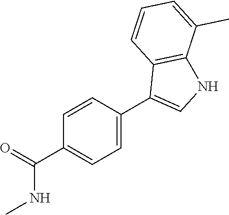 [SMILES: CNC(=O)c1ccc(-c2c[nH]c3c(C)cccc23)cc1]

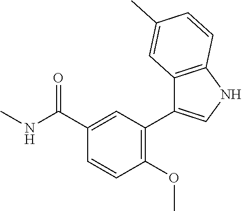 [SMILES: CNC(=O)c1ccc(OC)c(-c2c[nH]c3ccc(C)cc23)c1]

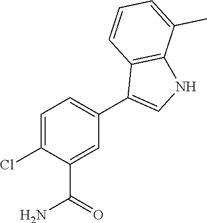 [SMILES: Cc1cccc2c(-c3ccc(Cl)c(C(N)=O)c3)c[nH]c12]

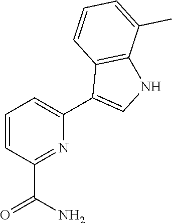 [SMILES: Cc1cccc2c(-c3cccc(C(N)=O)n3)c[nH]c12]

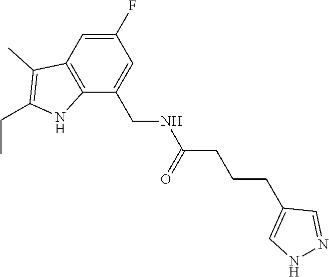 [SMILES: CCc1[nH]c2c(CNC(=O)CCCc3cn[nH]c3)cc(F)cc2c1C]

In certain aspects, the compound is:

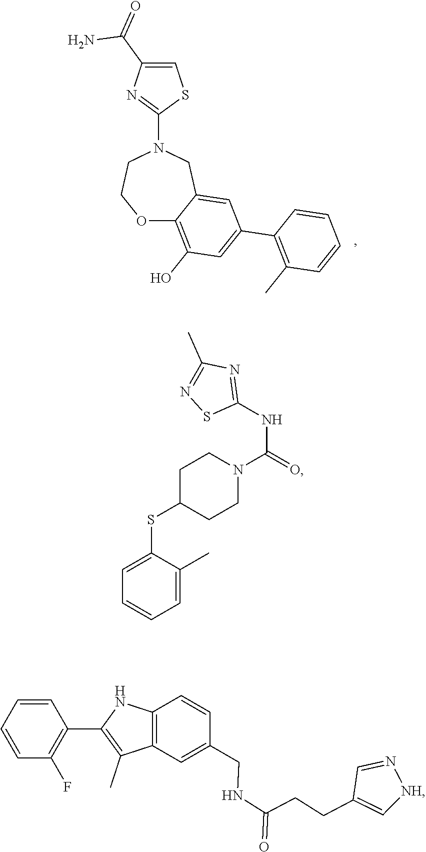 [SMILES: Cc1ccccc1-c1cc(O)c2c(c1)CN(c1nc(C(N)=O)cs1)CCO2 ; Cc1nsc(NC(=O)N2CCC(Sc3ccccc3C)CC2)n1 ; Cc1c(-c2ccccc2F)[nH]c2ccc(CNC(=O)CCc3cn[nH]c3)cc12]

or any combination thereof.

### III. Pharmaceutical Compositions and Methods of Use

Aspects of the disclosed compounds modulate AR-mediated activity. Some aspects of the disclosed compounds are useful for inhibiting or treating prostate cancer. In some aspects, AR is contacted with an effective amount of a compound disclosed herein, thereby inhibiting androgen receptor-coactivator (AR-CoA) protein-protein interaction (PPI) complexes, inhibiting prostate specific antigen (PSA) expression in prostate epithelial cells and/or prostate cancer cells, inhibiting PSA secretion by prostate epithelial cells and/or prostate cancer cells, inhibiting AR-mediated PSA promoter-driven transcription in prostate cancer cells, inhibiting androgen receptor splice variant 7 (AR-V7)-mediated PSA promoter driven transcription in prostate cancer cells, inhibiting ubiquitin conjugating enzyme E2 C (UBE2C) promoter-driven transcription in prostate cancer cells, inhibiting growth of prostate cancer cells, or any combination thereof. Inhibiting AR-CoA PPI complexes may include reducing formation of AR-CoA PPI complexes and/or disrupting formed AR-CoA PPI complexes. In some aspects, the CoA comprises transcriptional intermediary factor 2 (TIF2), steroid receptor coactivator (SRC1), or a combination thereof.

In any of the foregoing or following aspects, contacting may be performed in vivo. In some implementations, contacting in vivo comprises administering the effective amount of the compound to a subject.

In some aspects, a method for treating prostate cancer in a subject comprises administering to the subject a therapeutically effective amount of a compound as disclosed herein. In certain implementations, the prostate cancer is castration-resistant prostate cancer, such as metastatic CRPC. In some examples, the compound is orally administered, such as administered in an oral pharmaceutical composition. In any of the foregoing or following aspects, the method of treatment is used in combination with androgen deprivation therapy. In any of the foregoing or following aspects, the compound may be administered with another therapeutic agent. For example, the compound may be co-administered with abiraterone, enzalutamide, apalutamide, darolutamide, bicalutamide, flutamide, radium-223, olaparib, rcaparib, docetaxel, sipuleucel-T, or any combination thereof.

The compounds disclosed herein can be included in a pharmaceutical composition for administration to a subject. The pharmaceutical compositions for administration to a subject can include at least one further pharmaceutically acceptable additive such as carriers, thickeners, diluents, buffers, preservatives, surface active agents and the like in addition to the molecule of choice. Pharmaceutical compositions can also include one or more additional active ingredients such as antimicrobial agents, anti-inflammatory agents, anesthetics, and the like. The pharmaceutically acceptable carriers useful for these formulations are conventional. Remington: The Science and Practice of Pharmacy, The University of the Sciences in Philadelphia, Editor, Lippincott, Williams, & Wilkins, Philadelphia, PA, 21^{st} Edition (2005), describes compositions and formulations suitable for pharmaceutical delivery of the compounds disclosed herein and additional pharmaceutical agents.

The pharmaceutical compositions may be in a dosage unit form such as an injectable fluid, an oral delivery fluid (e.g., a solution or suspension), a nasal delivery fluid (e.g., for delivery as an aerosol or vapor), a semisolid form (e.g., a topical cream), or a solid form such as powder, pill, tablet, or capsule forms.

In general, the nature of the carrier will depend on the particular mode of administration being employed. For instance, parenteral formulations usually contain injectable fluids that include pharmaceutically and physiologically acceptable fluids such as water, physiological saline, balanced salt solutions, aqueous dextrose, glycerol or the like as a vehicle. For solid compositions (for example, powder, pill, tablet, or capsule forms), conventional non-toxic solid carriers can include, for example, pharmaceutical grades of mannitol, lactose, starch, or magnesium stearate. In addition to biologically-neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

The agents disclosed herein can be administered to subjects by a variety of mucosal administration modes, including by oral, rectal, intranasal, intrapulmonary, or transdermal delivery, or by topical delivery to other surfaces. Optionally, the agents can be administered by non-mucosal routes, including by intramuscular, subcutaneous, intravenous, intra-arterial, intra-articular, intraperitoneal, intrathecal, intracerebroventricular, or parenteral routes. In other alternative aspects, the agents can be administered ex vivo by direct exposure to cells, tissues or organs originating from a subject.

To formulate the pharmaceutical compositions, the agents can be combined with various pharmaceutically acceptable additives, as well as a base or vehicle for dispersion of the compound. Desired additives include, but are not limited to, pH control agents, such as arginine, sodium hydroxide, glycine, hydrochloric acid, citric acid, and the like. In addition, local anesthetics (for example, benzyl alcohol), isotonizing agents (for example, sodium chloride, mannitol, sorbitol), adsorption inhibitors (for example, Tween 80 or Miglyol 812), solubility enhancing agents (for example, cyclodextrins and derivatives thereof), stabilizers (for example, serum albumin), and reducing agents (for example, glutathione) can be included. Adjuvants, such as aluminum hydroxide (for example, Amphogel, Wyeth Laboratories, Madison, NJ), Freund's adjuvant, MPL™ (3-O-deacylated monophosphoryl lipid A; Corixa, Hamilton, IN) and IL-12 (Genetics Institute, Cambridge, MA), among many other suitable adjuvants well known in the art, can be included in the compositions. When the composition is a liquid, the tonicity of the formulation, as measured with reference to the tonicity of 0.9% (w/v) physiological saline solution taken as unity, is typically adjusted to a value at which no substantial, irreversible tissue damage will be induced at the site of administration. Generally, the tonicity of the solution is adjusted to a value of about 0.3 to about 3.0, such as about 0.5 to about 2.0, or about 0.8 to about 1.7.

The agents can be dispersed in a base or vehicle, which can include a hydrophilic compound having a capacity to disperse the compound, and any desired additives. The base can be selected from a wide range of suitable compounds, including but not limited to, copolymers of polycarboxylic acids or salts thereof, carboxylic anhydrides (for example, maleic anhydride) with other monomers (for example, methyl (meth)acrylate, acrylic acid and the like), hydrophilic vinyl polymers, such as polyvinyl acetate, polyvinyl alcohol, polyvinylpyrrolidone, cellulose derivatives, such as hydroxymethylcellulose, hydroxypropylcellulose and the like, and natural polymers, such as chitosan, collagen, sodium alginate, gelatin, hyaluronic acid, and nontoxic metal salts thereof. Often, a biodegradable polymer is selected as a base or vehicle, for example, polylactic acid, poly(lactic acid-glycolic acid) copolymer, polyhydroxybutyric acid, poly(hydroxybutyric acid-glycolic acid) copolymer and mixtures thereof. Alternatively or additionally, synthetic fatty acid esters such as polyglycerin fatty acid esters, sucrose fatty acid esters and the like can be employed as vehicles. Hydrophilic polymers and other vehicles can be used alone or in combination, and enhanced structural integrity can be imparted to the vehicle by partial crystallization, ionic bonding, cross-linking and the like. The vehicle can be provided in a variety of forms, including fluid or viscous solutions, gels, pastes, powders, microspheres and films for direct application to a mucosal surface.

The agents can be combined with the base or vehicle according to a variety of methods, and release of the agents can be by diffusion, disintegration of the vehicle, or associated formation of water channels. In some circumstances, the agent is dispersed in microcapsules (microspheres) or nanocapsules (nanospheres) prepared from a suitable polymer, for example, isobutyl 2-cyanoacrylate (see, for example, Michael et al., J. Pharmacy Pharmacol. 43:1-5, 1991), and dispersed in a biocompatible dispersing medium, which yields sustained delivery and biological activity over a protracted time.

The compositions of the disclosure can alternatively contain as pharmaceutically acceptable vehicles substances as required to approximate physiological conditions, such as pH adjusting and buffering agents, tonicity adjusting agents, wetting agents and the like, for example, sodium acetate, sodium lactate, sodium chloride, potassium chloride, calcium chloride, sorbitan monolaurate, and triethanolamine oleate. For solid compositions, conventional nontoxic pharmaceutically acceptable vehicles can be used which include, for example, pharmaceutical grades of mannitol, lactose, starch, magnesium stearate, sodium saccharin, talcum, cellulose, glucose, sucrose, magnesium carbonate, and the like.

Pharmaceutical compositions for administering the agents can also be formulated as a solution, microemulsion, or other ordered structure suitable for high concentration of active ingredients. The vehicle can be a solvent or dispersion medium containing, for example, water, ethanol, polyol (for example, glycerol, propylene glycol, liquid polyethylene glycol, and the like), and suitable mixtures thereof. Proper fluidity for solutions can be maintained, for example, by the use of a coating such as lecithin, by the maintenance of a desired particle size in the case of dispersible formulations, and by the use of surfactants. In many cases, it will be desirable to include isotonic agents, for example, sugars, polyalcohols, such as mannitol and sorbitol, or sodium chloride in the composition. Prolonged absorption of the compound can be brought about by including in the composition an agent which delays absorption, for example, monostearate salts and gelatin.

In certain aspects, the agents can be administered in a time release formulation, for example in a composition which includes a slow release polymer. These compositions can be prepared with vehicles that will protect against rapid release, for example a controlled release vehicle such as a polymer, microencapsulated delivery system or bioadhesive gel. Prolonged delivery in various compositions of the disclosure can be brought about by including in the composition agents that delay absorption, for example, aluminum monostearate hydrogels and gelatin. When controlled release formulations are desired, controlled release binders suitable for use in accordance with the disclosure include any biocompatible controlled release material which is inert to the active agent and which is capable of incorporating the compound and/or other biologically active agent. Numerous such materials are known in the art. Useful controlled-release binders are materials that are metabolized slowly under physiological conditions following their delivery (for example, at a mucosal surface, or in the presence of bodily fluids). Appropriate binders include, but are not limited to, biocompatible polymers and copolymers well known in the art for use in sustained release formulations. Such biocompatible compounds are non-toxic and inert to surrounding tissues, and do not trigger significant adverse side effects, such as nasal irritation, immune response, inflammation, or the like. They are metabolized into metabolic products that are also biocompatible and easily eliminated from the body.

Exemplary polymeric materials for use in the present disclosure include, but are not limited to, polymeric matrices derived from copolymeric and homopolymeric polyesters having hydrolyzable ester linkages. A number of these are known in the art to be biodegradable and to lead to degradation products having no or low toxicity. Exemplary polymers include polyglycolic acids and polylactic acids, poly(DL-lactic acid-co-glycolic acid), poly(D-lactic acid-co-glycolic acid), and poly(L-lactic acid-co-glycolic acid). Other useful biodegradable or bioerodable polymers include, but are not limited to, such polymers as poly(epsilon-caprolactone), poly(epsilon-caprolactone-CO-lactic acid), poly(epsilon.-caprolactone-CO-glycolic acid), poly(beta-hydroxy butyric acid), poly(alkyl-2-cyanoacrilate), hydrogels, such as poly(hydroxyethyl methacrylate), polyamides, poly(amino acids) (for example, L-leucine, glutamic acid, L-aspartic acid and the like), poly(ester urea), poly(2-hydroxyethyl DL-aspartamide), polyacetal polymers, polyorthoesters, polycarbonate, polymaleamides, polysaccharides, and copolymers thereof. Many methods for preparing such formulations are well known to those skilled in the art (see, for example, Sustained and Controlled Release Drug Delivery Systems, J. R. Robinson, ed., Marcel Dekker, Inc., New York, 1978). Other useful formulations include controlled-release microcapsules (U.S. Pat. Nos. 4,652,441 and 4,917,893), lactic acid-glycolic acid copolymers useful in making microcapsules and other formulations (U.S. Pat. Nos. 4,677,191 and 4,728,721) and sustained-release compositions for water-soluble peptides (U.S. Pat. No. 4,675,189).

The phannaceutical compositions of the disclosure typically are sterile and stable under conditions of manufacture, storage and use. Sterile solutions can be prepared by incorporating the compound in the required amount in an appropriate solvent with one or a combination of ingredients enumerated herein, as required, followed by filtered sterilization. Generally, dispersions are prepared by incorporating the compound and/or other biologically active agent into a sterile vehicle that contains a basic dispersion medium and the required other ingredients from those enumerated herein. In the case of sterile powders, methods of preparation include vacuum drying and freeze-drying which yields a powder of the compound plus any additional desired ingredient from a previously sterile-filtered solution thereof. The prevention of the action of microorganisms can be accomplished by various antibacterial and antifungal agents, for example, parabens, chlorobutanol, phenol, sorbic acid, thimerosal, and the like.

In accordance with the various treatment methods of the disclosure, the agent can be delivered to a subject in a manner consistent with conventional methodologies associated with management of the disorder for which treatment or prevention is sought. In accordance with the disclosure herein, a prophylactically or therapeutically effective amount of the agent is administered to a subject in need of such treatment for a time and under conditions sufficient to prevent, inhibit, and/or ameliorate a selected disease or condition or one or more symptom(s) thereof.

The administration of the agent can be for either prophylactic or therapeutic purpose. When provided prophylactically, the agent is provided in advance of any symptom. The prophylactic administration of the agents serves to prevent or ameliorate any subsequent disease process. When provided therapeutically, the compound is provided at (or shortly after) the onset of a symptom of disease or infection.

For prophylactic and therapeutic purposes, the agent can be administered to the subject by the oral route or in a single bolus delivery, via continuous delivery (for example, continuous transdermal, mucosal or intravenous delivery) over an extended time period, or in a repeated administration protocol (for example, by an hourly, daily or weekly, repeated administration protocol). The therapeutically effective dosage of the agent can be provided as repeated doses within a prolonged prophylaxis or treatment regimen that will yield clinically significant results to alleviate one or more symptoms or detectable conditions associated with a targeted disease or condition as set forth herein. Determination of effective dosages in this context is typically based on animal model studies followed up by human clinical trials and is guided by administration protocols that significantly reduce the occurrence or severity of targeted disease symptoms or conditions in the subject. Suitable models in this regard include, for example, murine, rat, avian, porcine, feline, non-human primate, and other accepted animal model subjects known in the art. Alternatively, effective dosages can be determined using in vitro models. Using such models, only ordinary calculations and adjustments are required to determine an appropriate concentration and dose to administer a therapeutically effective amount of the compound (for example, amounts that are effective to elicit a desired immune response or alleviate one or more symptoms of a targeted disease). In alternative aspects, an effective amount or effective dose of the agents may simply inhibit or enhance one or more selected biological activities correlated with a disease or condition, as set forth herein, for either therapeutic or diagnostic purposes.

The actual dosage of the agents will vary according to factors such as the disease indication and particular status of the subject (for example, the subject's age, size, fitness, extent of symptoms, susceptibility factors, and the like), time and route of administration, other drugs or treatments being administered concurrently, as well as the specific pharmacology of the agent for eliciting the desired activity or biological response in the subject. Dosage regimens can be adjusted to provide an optimum prophylactic or therapeutic response. A therapeutically effective amount is also one in which any toxic or detrimental side effects of the agent is outweighed in clinical terms by therapeutically beneficial effects. A non-limiting range for a therapeutically effective amount of an agent within the methods and formulations of the disclosure is about 0.01 mg/kg body weight to about 20 mg/kg body weight, such as about 0.05 mg/kg to about 5 mg/kg body weight, or about 0.2 mg/kg to about 2 mg/kg body weight. Dosage can be varied by the attending clinician to maintain a desired concentration at a target site (for example, the lungs or systemic circulation). Higher or lower concentrations can be selected based on the mode of delivery, for example, trans-epidermal, rectal, oral, pulmonary, or intranasal delivery versus intravenous or subcutaneous delivery. Dosage can also be adjusted based on the release rate of the administered formulation, for example, of an intrapulmonary spray versus powder, sustained release oral versus injected particulate or transdermal delivery formulations, and so forth.

### IV. Examples

### Materials and Methods

Reagents: Formaldehyde, dihydrotestosterone (DHT), flutamide, bicalutamide, and enzalutamide were purchased from Sigma-Aldrich (St. Louis, MO). Hoechst 33342 was purchased from Invitrogen (Carlsbad, CA). Dimethyl sulfoxide (DMSO) (99.9% high-performance liquid chromatography grade, under argon) was from Alfa Aesar (Ward Hill, MA). Dulbecco's Mg2+ and Ca2+ free phosphate-buffered saline (PBS) was purchased from Corning (Tewksbury, MA). The AlphaScreen® Histidine (Nickel Chelate) Detection Kit, 500 assay points was purchased from Perkin Elmer (Waltham, MA), Geneticin™ Selective Antibiotic (G418 Sulfate) powder, was purchased from Fisher Scientific (Pittsburgh, PA). FuGENE™ 6 and FuGENE™ HD transfection Reagents were purchased from Promega (Madison, WI). Bright-Go™ Luciferase Assay System was purchased from Promega. Dihydrotestosterone [1,2,4,5,6,7-3H(N)]-(5 alpha-ANDROSTAN-17 beta-3-ol) was purchased from Perkin Elmer.

Cell Lines and Tissue Culture: PC-3 and DU-145 cells were provided by the National Cancer Institute (NCI) as part of the NCI 60 tumor cell line panel. LNCaP (CRL-1740) and 22Rv1 (CRL-2505) cells were obtained from the American Type Culture Collection (Manassas, VA). C4-2 cells were purchased from UroCor (Oklahoma City, OK) and kindly provided by Dr. Zhou Wang (University of Pittsburgh, Pittsburgh, PA). All the prostate cancer cell lines were maintained in RPMI 1640 medium with 2 mM L-glutamine (Invitrogen, Carlsbad, CA) supplemented with 10% fetal bovine serum (Gemini Bio-Products, West Sacramento, CA), and 100 U/mL penicillin and streptomycin (Invitrogen, Carlsbad, CA). PC3 cells that stably express AR-V7-GFP were kindly provided by Dr. Michael Mancini in the Departments of Molecular and Cellular Biology, and Pharmacology and Chemical Biology, Baylor College of Medicine, Houston, TX. PC3-AR-V7-GFP cells were maintained in DME/F12 (Gibco, Gaithersburg, MD) and supplemented with 10% FBS and 500 μg/mL Geneticin (G418) (Fisher Scientific). The U-2 OS osteosarcoma cell line was acquired from American Type Culture Collection and was maintained in McCoy's 5A medium with 2 mM L-glutamine (Invitrogen, Carlsbad, CA) supplemented with 10% fetal bovine serum (Gemini Bio-Products, West Sacramento, CA), and 100 U/mL penicillin and streptomycin (Invitrogen, Carlsbad, CA). HEK 293 cells (CRL-1537) were purchased from the American Type Culture Collection (Manassas, VA) and were maintained in DMEM (Cellgro™ 10013CV cell culture medium) (Corning, Tewksbury, MA) with 2 mM L-glutamine (Invitrogen) that was supplemented with 10% fetal bovine serum (Gemini Bio-products), and 100 U/mL penicillin and streptomycin (Invitrogen). All cell lines were maintained in a humidified incubator at 37° C., 5% CO₂, and 95% humidity.

Compounds and Compound Handling: To determine 50% inhibition (IC₅₀) or growth inhibition (GI₅₀) concentrations in each assay, 10-point two-fold or three-fold serial dilutions of test compounds in 100% DMSO were performed using a 384-well P30 dispensing head on the Janus MDT automated liquid handling platform (Perkin Elmer, Waltham, MA). Daughter plates containing 2 μL of the serially diluted compounds in DMSO were prepared and replicated from 384-well serial dilution master plates using a Janus MDT platform outfitted with a 384-well transfer head. Aluminum adhesive plate seals were applied, and plates were stored at −20° C. For bioassay testing, daughter plates were withdrawn from −20° C. storage, thawed to ambient temperature, and centrifuged for 1 min at 100×g, and plate seals were removed before 38 μL of serum-free media (SFM) was transferred into wells using a Matrix™ pipettor (ThermoFisher, Waltham, MA), to generate an intermediate stock concentration of validation compounds ranging from 0.977 to 500 μM (5.0% DMSO). Diluted compounds were mixed by repeated aspiration and dispensation using a 384-well P30 dispensing head on the Janus MDT platform and then, 5 μL of diluted compounds was transferred to assay plate wells to provide a final concentration range from 0.0977 to 50 μM (0.5% DMSO).

AR-TIF2 Protein-Protein Interaction Biosensor Assay: The AR-TIF2 PPIB HCS assay was performed in U-2 OS osteosarcoma cells as described previously (Fancher et al., Assay Drug Dev Technol. 2016, 14:453-477; Fancher et al., Assay Drug Dev Technol. 2018, 16:297-319; Hua et al., Assay Drug Dev Technol. 2014, 12:395-418; Hua et al., Methods Mol Biol. 2018, 1683:211-227). Briefly, U-2 OS cells were coinfected with recombinant adenovirus biosensor expression constructs and seeded at 2,500 cells per well in 384-well collagen-coated microplates (Greiner Bio-One #781956) and plates were incubated overnight at 37° C. in 5% CO₂ and 95% humidity. To block DHT-induced AR-TIF2 PPI formation, assay plates were pre-incubated with compounds for 3 h prior to exposure to 25 nM DHT for 90 minutes. To disrupt pre-existing AR-TIF2 PPI complexes, assay plates were pre-incubated with 25 nM DHT for 90 minutes prior to the transfer of compounds for an additional 3 h incubation. Maximum plate control wells (n=32, columns 1 & 2) were exposed to 25 nM DHT and ≤0.25% DMSO, and minimum plate control wells (n=32, columns 23 & 24) were treated with ≤0.25% DMSO. Diluted compounds, DHT or DMSO (5 μL) were transferred at the indicated concentrations as described above. After the appropriate time, assay plates were fixed by transfer of 50 μL of pre-warmed (37° C.) 7.4% formaldehyde and 2 μg/mL Hoechst 33342 in PBS and incubation at room temperature for 30 minutes. Liquid was aspirated, plates were washed twice with 85 μL PBS, leaving the final wash in the plate. Plates were sealed with adhesive aluminum plate seals, and fluorescent images of three fields of view were acquired in the DAPI (Hoechst stained nuclei), FITC (TIF2-GFP) and Texas Red (AR-RFP) channels on an ImageXpress® Micro (IXM) automated HCS platform (Molecular Devices LLC, Sunnyvale, CA) using a 10× Plan Fluor 0.3 NA objective. Images were analyzed using the Translocation Enhanced (TE) image analysis module of the MetaXpress® software (Molecular Devices, LLC, San Jose, CA) as described previously (Fancher et al., Assay Drug Dev Technol. 2016, 14:453-477; Fancher et al., Assay Drug Dev Technol. 2018, 16:297-319; Hua et al., Assay Drug Dev Technol. 2014, 12:395-418; Hua et al., Methods Mol Biol. 2018, 1683:211-227).

TIF2 and SRC1 Mammalian 2-Hybrid Assays: The 5×GAL4-TATA-luciferase reporter plasmid was a gift from Dr. Richard Maurer from the Oregon Health and Science University (Gonzalez et al., Cell 1989, 59:675-80), and constructs pGAL4-hAR-658-919 (AR-LBD amino acids 658-919 expressed as a fusion protein with Gal4-DBD) (Askew et al., J Biol Chem 2007, 282:25801-16), pVP16-SRC1 (full-length SRC1 expressed as a fusion with VP16 activation domain) (He et al., J Biol Chem 1999, 274:37219-25) and pVP16-Empty vectors were kindly provided to us by Dr. Elizabeth Wilson, from UNC Chapel Hill. pVP16-TIF2 was generated as described previously (Fancher et al., Assay Drug Dev Technol. 2019, 17:364-386). HEK 293 cells were transiently co-transfected with 5 ng of pGal4-AR-LBD, 10 ng of either pVP16-TIF2 or pVP16-SRC1, and 20 ng of the 5×Gal4-TATA-Luc reporter as described previously (Ibid.). HEK 293 cells were bulk co-transfected with the three plasmids that had been individually incubated with FuGENE® 6 transfection agent (Fugent LLC, Middleton, WI) at a 3:1 ratio for 25 min at room temperature (RT) in serum free media (SFM) and then combined with HEK 293 cells that were suspended in DMEM (Cellgro10013CV) with 2 mM L-glutamine (Invitrogen) that was supplemented with 10% fetal bovine serum, and 5,000 cells in a volume of 40 μL were seeded into the wells of white opaque 384-well assay plates (Greiner Bio-one, #781080) and cultured overnight at 37° C., 5% CO₂, and 95% humidity. 24 h post cell seeding into assay plates, 5 μL of serially diluted compounds were transferred to assay wells and plates were incubated at 37° C., 5% CO₂, and 95% humidity for 3 h before 5 μL of 0.25 μM DHT (25 nM final) was transferred into each well, and the assay plates were returned to the incubator for an additional 24 h. 25 μL of BrightGlo® reagent (Promega, Madison, WI) was added to the plate and the relative luminescence units (RLUs) were captured on a SpectraMax® M5e microtiter plate reader (Molecular Devices, LLC, San Jose, CA).

Prostate Specific Antigen (PSA)-6.1 Luciferase Reporter Assay in the C4-2 CRPC Cell Line: The PSA-6.1-Luc luciferase reporter plasmid was provided by Dr. Zhou Wang in the Urology department of the University of Pittsburgh Cancer Institute. The PSA-6.1-Luc reporter is controlled by a fragment of the PSA promoter that contains at least three AREs. The PSA-6.1-Luc plasmid (12 ng/well) was combined with FuGENE® 6 transfection agent at a ratio 6:1 in SFM and incubated for 25 minutes at room temperature before being combined with C4-2 cells suspended in RPMI 1640 media containing 1% penicillin-streptomycin, 1% L-glutamine, and 10% FBS. Transfected cells were then seeded into white opaque 384-well assay plates (Greiner Bio-one, #781080) at 6,000 cells per well in a volume of 30 μL and incubated in 5% CO₂, 37° C., and 95% humidity for 24 h. After 24 h, 5 μL of compounds were transferred to the wells and then 5 μL of DHT (50 nM final in well) in SFM was transferred to each well and the assay plates were returned to the incubator for an additional 24 h before 20 μL of Bright-Glo™ luciferase reagent (Promega, Madison, WI) was added to the wells and the relative light units (RLUs) were captured on a SpectraMax® M5e plate reader (Molecular Devices LLC, Sunnyvale, CA) as described previously (Fancher et al., Assay Drug Dev Technol. 2016, 14:453-477; Fancher et al., Assay Drug Dev Technol. 2019, 17:364-386).

PSA6.1 Promoter Driven Luciferase Reporter Assay in PC3-AR-V7-GFP Cells: PC3-AR-V7-GFP cells were bulk transfected with a mixture of FuGENE® HD transfection agent and the PSA-6.1-Luc reporter plasmid (20 ng/well) combined at a 3:1 (μL:μg) ratio in Opti-MEM™ medium (Gibco, Gaithersburg, MD) that had been incubated for 25 min at RT before being added to PC3-AR-V7-GFP cells that were suspended in RPMI 1640 (Gibco) media containing 1% L-glutamine (Invitrogen), and 10% fetal bovine serum (Gemini Bio-products). Bulk transfected PC3-AR-V7-GFP cells were seeded into white opaque 384-well assay plates (Greiner Bio-one, #781080) at 3,000 cells per well in a volume of 40 μL and incubated at 5% CO₂, 37° C., and 95% humidity for 24 h. After 24 h, 5 μL of compounds were transferred to assay wells and the plates were returned to the incubator for an additional 24 h before 25 μL of BrightGlo® luciferase reagent (Promega) was added to the wells and the RLU's were captured on a SpectraMax® M5e plate reader (Molecular Devices LLC) as described previously (Fancher et al., Assay Drug Dev Technol. 2019, 17:364-386).

UBE2C Promoter Driven Luciferase Reporter Assay in PC3-AR-V7-GFP Cells: The pGL4.28-UBE2C 20bpX3 luciferase reporter plasmid (Xu et al., Cancer Res 2015, 75:3663-71) was provided by Dr. Yan Dong from Tulane University. FuGENE® HD transfection agent and the UBE2C-Luc plasmid (10 ng/well) were combined at a 3:1 (μL:μg) ratio, in Opti-MEM™ medium and incubated for 25 min at RT before being added to PC3-AR-V7-GFP cells that were suspended in RPMI 1640 (Gibco) media containing 1% L-glutamine (Invitrogen), and 10% fetal bovine serum (Gemini Bio-products). Bulk transfected PC3-AR-V7-GFP cells were seeded into white opaque 384-well assay plates (Greiner Bio-one, #781080) at a density of 3,000 cell per well in a volume of L and incubated at 5% CO₂, 37° C., and 95% humidity for 24 h. After 24 h, 10 μL of compounds were transferred to assay wells and the plates were returned to the incubator for an additional 24 h before 25 μL of BrightGlo® luciferase reagent (Promega) was added to the wells and the RLU's were captured on a SpectraMax® M5e plate reader (Molecular Devices LLC) as described previously (Ibid.).

Western and Dot Blotting Assay to Measure PSA Expression and Secretion in C4-2 Cells: To determine cellular PSA expression levels, C4-2 cells were suspended in RPII 1640 media containing 10% charcoal stripped FBS and seeded at 2-4×10′ cells/well in Costar 12-well plates (Corning, #3513) that were incubated overnight at 5% CO₂, 37° C., and 95% humidity. C4-2 monolayers were washed 1× with serum free RPMI 1640 medium, and then 900 μL of Opti-MEM™ medium (Gibco, Gaithersburg, MD) containing either DMSO (0.2%) or compounds (20 μM, 0.2% DMSO) were added to wells and incubated for 3 h before addition of 100 μL of Opti-MEM™ medium with or without 100 nM DHT (10 nM final). After a 24 h incubation at 5% CO₂, 37° C., and 95% humidity conditioned media was collected and used for dot bots (see below) and C4-2 cell monolayers were washed once with PBS then lysed in 100 μL of cell lysis buffer (500 mM NaCl, 1% NP-40, 1× protease inhibitor cocktail in PBS), transferred to PCR tubes and placed on ice for an additional 30 min. Cell lysate protein concentrations were determined in a bicinchoninic acid (BCA) assay. Equal amounts of cell protein were mixed with SDS-PAGE sample buffer and placed in a heat block at 100° C. for 5 min. The protein constituents of C4-2 cells were separated by SDS-PAGE on 10% separating gels, transferred to nitrocellulose membranes and western blots were probed overnight at 4° C. with a 1:1000 dilution of a rabbit anti-hPSA (Cell Signaling, Danvers, MA) primary antibody in Tris-buffered saline (TBS) Tween 20 (TBST) containing 5% non-fat milk. Membranes were washed 3× in TBST for 10 min, then incubated for 1 h at room temperature with a 1:10,000 dilution of the goat anti-rabbit IgG horse radish peroxidase (HRP) conjugated secondary antibody (Invitrogen, Carlsbad, CA) in TBST containing 5% non-fat milk. Western blots were then washed 3× in TBST and developed with Pierce enhanced chemiluminescence (ECL) western blotting substrate (Thermo Fisher Scientific, Waltham, MA). Images of western blot ECL bands were acquired on an iBright™ 1500 imaging system (Thermo Fisher Scientific, Waltham, MA) and quantified by iBright™ image analysis software.

To determine PSA secretion levels, C4-2 cells were seeded at 1.4×10⁵ cells/well in 12-well plates and treated as described above for PSA cell expression experiments. After 3 h compound exposure and 24 h DHT treatment at 5% CO₂, 37° C., and 95% humidity, conditioned media was collected from wells, transferred to tubes, and centrifuged at 14,000 RPM (18,800×g) for 15 min. 500 μL of conditioned media supernatant was added to the wells of 96-well to Bio-blot apparatus (BioRad, Hercules, CA) containing a nitrocellulose membrane and was allowed to pass through and attach to the membrane under gravity for 3-4 h at room temperature. The membrane was washed 1× with 500 μL TBS under vacuum, blocked with 1% BSA in TBST for 1 h, and then incubated overnight at 4° C. with the primary rabbit anti-hPSA antibody (Cell Signaling, Danvers, MA) diluted 1:1000 in TBST plus 1% BSA. Dot blots were washed 3× in 10 mL of TBST for 10 min, then incubated for 1 h with secondary goat anti-rabbit-IgG HRP conjugated antibody (Invitrogen, Carlsbad, CA) diluted 1:10,000 in TBST plus 1% Bovine Serum Albumin (BSA), washed 3× with 10 nL of TBST for 10 min, and then developed with Pierce ECL western blotting substrate. Images of ECL dot blots were acquired on an iBright™ 1500 imaging system and quantified by iBright™ image analysis software (ThermoFisher Scientific, Waltham, MA).

AR-LBD::TIF2-Box I-LXXLL-Peptide Binding Assay: The pET28a-AR-LBD (622-919) construct (Feau et al., J Biomol Screen 2009, 14:43-8) was a gift from Dr. Fletterick and Dr. Nguyen of University of California San Francisco. Biotinylated (Biotin-HN-CKKKENALLRYLLDKDDTKD-CONH₂; SEQ ID NO: 1) and non-biotinylated TIF2-box-III (738-756) peptide (H₂N-CKKKENALLRYLLDKDDTKD-CONH₂; SEQ ID NO: 2) were synthesized by the Peptide & Peptoid Synthesis Facility, at the University of Pittsburgh Health Sciences Core Research Facilities. ALPHAScreen® streptavidin donor beads (SA-DB) and nickel chelate acceptor beads (Ni-AB) were purchased from Perkin Elmer (Waltham, MA). The assay was performed in 384-well white opaque plates (Greiner BioOne, #781080). 150 nM of biotinylated TIF2-box III peptide was incubated with 5 μg/μL SA-BD, and His₆-AR-LBD (400 ng/well) was incubated with 10 μM DHT plus 5 μg/μL Ni-ABs for 30 min at room temperature in the dark. 18 μL of the SA-DB bound biotinylated TIF2 peptide mixture was added to the assay plate before 5 μL of compounds were transferred into assay wells and 27 μL of the AlphaScreen® donor and acceptor B bead mixture was added to the plate. 32 wells containing 0.5% DMSO provided maximum controls and 32 wells containing a 500-fold excess of unlabeled TIF2-box-III (75 μM) were used as minimum controls. The combined head-protein-peptide-compound mixture was incubated for 1 h at room temperature in the dark, and then the RLU's were acquired at 520 nm after excitation at 680 nm on an EnVision® plate reader (Perkin Elmer, Waltham, MA) as described previously (Fancher et al., Assay Drug Dev Technol. 2019, 17:364-386).

H³-DHT Radioligand Binding Assay: The His₆-AR-LBD H³-DHT competition binding assay has been described previously (Fancher et al., Assay Drug Dev Technol. 2016, 14:453-477; Fancher et al., Assay Drug Dev Technol. 2019, 17:364-386). Briefly, 96-well Cu²⁺-coated plates (ThermoFisher) were incubated overnight at 4° C. with 5 g per well His₆-AR-LBD in 100 μL of PBS. Unbound His₆-AR-LBD was aspirated, the plate was washed 3× with 100 μL of 0.05% Tween 20 in PBS and then blocked with 100 μL of 1 mg/mL BSA in PBS for 1 h. After three more washes with 100 μL of PBS and 0.05% Tween 20, 40 μL of PBS was added to wells followed by 5 L each of diluted compounds and 100 nM H³-DHT transferred into the wells using a Matrix pipettor. Compounds were tested between 0.098 to 50 μM in the presence of 10 nM H³-DHT. After 1 h, compounds and H³-DHT were aspirated and washed 3× with 0.05% Tween 20 in PBS; 100 μL of Microscint™-20 micro-scintillation cocktail buffer (Perkin Elmer, Waltham, MA) was added to each well, plates were sealed with adhesive plastic covers; and the counts per minute (CPMs) were captured in a TopCount NXT microtiter plate reader (Perkin Elmer, Waltham, MA).

Prostate Cancer Cell Line Growth Inhibition Assays: The PC-3, DU-145, LNCaP, C4-2, and 22Rv1 PC cell line growth inhibition assays have been described previously (Fancher et al., Assay Drug Dev Technol. 2016, 14:453-477; Fancher et al., Assay Drug Dev Technol. 2019, 17:364-386). On day 1, each PC cell line was harvested, counted, and seeded into two 384-well assay plates, a time zero (T0) and a time 72 h (T72) plate. PC cell lines were all seeded at 1,000 cells per well in 45 μL of tissue culture media in uncoated white clear bottom 384-well assay plates (VWR, #82050-076) using a Matrix electronic multichannel pipette (Thermo Fisher Scientific, Waltham, MA) and cultured overnight at 37° C., 5% CO2, and 95% humidity. On day 2, 25 μL of the CellTiter-Glo® (CTG) (Promega Corporation, Madison, WI) detection reagent was dispensed into the wells of the TO assay plate using a Matrix electronic multichannel pipette, and the RLUs were captured on the SpectraMax® M5e (Molecular Devices LLC, Sunnyvale, CA) microtiter plate reader. Also on day 2, 5 μL of compounds were transferred into the test wells of the T72 384-well assay plates which were returned to the incubator for 72 h. Control wells received DMSO alone. On day 5, 25 μL of the CTG detection reagent was dispensed into the wells of the T72 assay plate using a Matrix electronic multichannel pipette, and the RLU's were captured on the SpectraMax® M5e microtiter plate reader platform.

Western Blotting Cellular Thermal Shift Assays for TIF2 and AR Target Engagement in C4-2 Cells: C4-2 cells were harvested by trypsinization, washed 1× by centrifugation at 270×g for 5 min and resuspension in PBS, counted, centrifugated at 270× g for 5 min and resuspended at 7×10⁶ cells per mL in Opti-MEM medium (Gibco, Gaithersburg, MD). 50 μL of C4-2 cell suspension (3.5×10⁵ cells) were then transferred to PCR tubes that were placed in a T-100 thermocycler (BioRad, Hercules, CA) and a 2° C. interval temperature step gradient from 37° C. to 53° C. was applied. Cells were maintained at each step of the temperature gradient for 5 min and then tubes were withdrawn and placed on ice. 50 μL of cell lysis buffer, 500 mM NaCl, 1% NP-40, 1× protease inhibitor cocktail in PBS were added to the heat shocked cell suspensions in PCR tubes and placed on ice for an additional 30 min. Cell lysates were then centrifuged at 14,000 RPM (18,800×g) at 4° C. for 15 min and supernatants were transferred to new tubes and protein concentrations were determined in a bicinchoninic acid (BCA) assay. 45 μL of cell lysis supernatants were mixed with 15 μL of 5×SDS-PAGE sample buffer and placed in a heat block at 100° C. for 5 min. The protein constituents of heat shocked C4-2 cell lysis supernatants were separated by SDS-PAGE on 8% separating gels, transferred to nitrocellulose membranes that were blocked for 1 h at room temperature in 5% non-fat milk in TBST, and then probed overnight at 4° C. with a 1:1000 dilution of either rabbit anti-AR (Cell Signaling, Danvers, MA) or rabbit anti-TIF2 (Bethyl Laboratories, Waltham, MA) primary antibodies in TBST containing 5% non-fat milk. Membranes were then washed 3× in TBST buffer for 10 min, then incubated with a 1:10,000 dilution of the goat anti-rabbit IgG HRP conjugated secondary antibody (Invitrogen, Carlsbad, CA) in TBST containing 5% non-fat milk for 1 h at room temperature. Western blots were then washed 3× in TBST buffer and developed with Pierce ECL western blotting substrate. Images of ECL western blots were acquired on an iBright™ 1500 imaging system and quantified using the iBright™ image analysis software.

AlphaScreen® Cellular Thermal Shift Assay (CETSA) for AR Target Engagement in C4-2 Cells: C4-2 cells were harvested by trypsinization, washed 1× by centrifugation at 270×g for 5 min and resuspension in PBS, counted, centrifugated at 270× g for 5 min and then resuspended at 3.125×10⁶ cells per mL in Opti-MEM medium (Gibco, Gaithersburg, MD). 32 μL of C4-2 cell suspension (1×10⁵ cells) were transferred to PCR tubes, and 4 μL of either DMSO (0.25% final) or compounds in DMSO were added and tubes were incubated for 1 h at 37° C., 5% CO2, and 95% humidity. Cells were then incubated with 4 μL of media or DHT (100 nM final) for 1 h at 37° C., 5% CO2, and 95% humidity before PCR tubes were placed in a T-100 thermocycler that was heated to 46 C and maintained for 5 min before 40 μL of 2× lysis buffer (2% Triton x-100, 100 mM NaCl, 1 mg/mL BSA, and protease inhibitor cocktail in PBS) was added and tubes were placed on ice for an additional 20 min. Cell lysates were then centrifuged at 14,800 RPM (21,000×g) at 4° C. for 20 min and the amount of soluble AR in supernatants was quantified in a modified version of an AlphaScreen® AR CETSA assay (Shaw et al., Sci Rep. 2018, 8:163-174) where one of the anti-AR antibodies was changed from the published protocol. Mouse anti-hAR (BD Biosciences, San Jose, CA) and rabbit anti-hAR (MilliporeSigma, Burlington, MA) were diluted 1:330 and 1:1000 fold respectively in PBS containing 0.5 mg/mL BSA and 4 μL of the combined diluted AR antibody pair were added to 4 μL of the cell lysate supernatant in a 384-well plate and incubated in the dark for 30 min at room temperature. To each well of the 384-well plate 4 μL of a combined solution of anti-mouse IgG Alpha Donor and anti-rabbit IgG (Fc specific) AlphaLISA® Acceptor beads (Perkin Elmer, Waltham, MA) suspended in 1× lysis buffer was added to yield a final donor and acceptor bead concentrations of 40 μg/mL and 10 μg/mL respectively. The bead-cell lysate-compound mixture was incubated overnight (16 h) at room temperature in the dark, and then RLU's were acquired at 520 nm after 680 nm excitation on an Envision plate reader (Perkin Elmer, Waltham, MA).

Molecular Docking Studies: A virtual screening pipeline of novel computational technologies was applied to dock the representative hit compounds S1-1, S2-6, and S3-11 to different AR structures using a variety of platforms to predict druggable sites, conduct pharmacophore-based interactive virtual screening, and the Smina version of AutoDock-Vina specially optimized to support high-throughput minimization and scoring (Koes el al., Nucleic Acids Res. 2012, 40:W387-392; Koes et al., PLoS One 2012 7′ Koes et al., J Chem Inf Model. 2013, 53:1893-1904). These methods have been prospectively validated both in terms of the accuracy of the predicted poses as well as ranking of those poses (Baumgartner et al., J Chem Inf Model. 2016, 56:1004-1012; Smith et al., J Chem Inf Model. 2016, 56:1022-1031). The poses presented are for the PDB 2AO6 crystal structure of the human androgen receptor ligand binding domain bound with TIF2 (iii) 740-753 peptide and R1881 (He et al., Trends Biochem Sci 2009, 34:579-588).

Data Processing, Visualization, Statistical Analysis and IC₅₀ Curve Fitting: In 384-well assays, DMSO minimum (n=32) and maximum (n=32) plate control wells were utilized to calculate signal-to-background ratios (S:B) and Z′-factor coefficient assay performance quality control statistics, and to normalize the signals of compound treated wells and to represent 0% and 100% respectively. For the 96-well AR-LBD H³-DHT radioligand binding assay, eight minimum and maximum plate control wells were utilized to normalize the data. For the PC cell line growth inhibition assays the DMSO control data from the TO and T72 assay plates was used to assess the dynamic range of the T0 to T72 cell growth, and to calculate S:B ratios and Z′-factor coefficient statistics for the assay signal window (T0 to T72). To normalize the 72 h compound exposure PC growth inhibition data, the signals from the compound treated wells were processed and expressed as % of the T72 DMSO plate controls. IC₅₀ and GI₅₀ values for each of the bioassays were calculated using GraphPad Prism 9 software to plot and fit data to curves using the Sigmoidal dose response variable slope equation Y=Bottom+[Top-Bottom]/[1+10\hat{ }(Log EC50−X)*HillSlope].

### Example 1

### Compound Screening

A positional AR-TFI2 protein-protein interaction biosensor (PPIB) assay was used to identify small molecules that inhibited DHT-induced formation of AR-TIF2 PPIs and/or disrupted pre-existing AR-TIF2 PPIs (Fancher et al., Assay Drug Dev Technol 2016, 14(8):453-477; Fancher et al., Assay Drug Dev Technol 2018, 16(6):297-319; Hua et al., Assay Drug Dev Technol 2014, 12:395-418; Hua et al., Methods Mol biol 2018, 1683:211-227). AR-LBD residues (662-919) were incorporated into one biosensor and TIF2 residues (725-840) containing the 3^{rd} NR box LXXLL motif into the second interacting biosensor partner (Id.) The AR-TIF2 PPIB recapitulates the orthosteric ligand (OSL)-induced translocation of AR from the cytoplasm into the nucleus where PPIs with TIF2 result in colocalization of both biosensors in the nucleolus. Using three libraries, a total of 143,535 compounds were screened—a 10,000-compound ChemDiv PPI focused diversity library, a 50,000-compound ChemBridge diversity library, and a 83,535-compound library provided by the NCI's Chemical Biology Consortium. The 10K and 50K diversity libraries were subsets of 142K and 810K parent libraries, enabling screening of a representative subset of >1×10⁶ compounds. AR-TIF2 PPI inhibitor and disruptor actives were confirmed in the HCS assay and counter screens designed to identify and exclude nuisance/interference or non-selective compounds (Fancher et al., Assay Drug Dev Technol 2018, 16(6):297-319). Fluorescent intensity HCS data was used to flag auto-fluorescent compounds, and a p53-hDM2 counter screen with the same biosensor design but different PPI partners was used to exclude assay format interfering compounds (Id.; Dudgeon et al., Assay Drug Dev Technol 2010, 8; 437-458; Dudgeon et al., J Biomol Screen 2010, 15:152-174). A GR nuclear translocation assay was used to exclude compounds that non-specifically blocked NR trafficking into nuclei (Fancher et al., Assay Drug Dev Technol 2018, 16(6):297-319; Daghestani, Assay Drug Dev Technol 2012, 10(1):46-60; Johnston el al., Assay Drug Dev Technol 2012, 10(5):432-456). An AR-GFP subcellular localization assay was used to exclude compounds that reduced AR expression and/or restricted its localization to the cytoplasm (Fancher et al., Assay Drug Dev Technol 2018, 16(6):297-319; Johnston et al., Assay Drug Dev Technol 2016, 14:226-239; Masodi et al., Mol Cancer Ther 2017, 16(10):2120-2129). Hits were structurally classified, clustered, and medicinal chemistry computational filters (PAINS/REOS) were used to exclude nuisance compounds and evaluate drug-like properties (Baell, J Med Chem 2010, 53(7):2719-2740; Baell, ACS Chem. Biol. 2018, 13:36-44; Johnston, Curr Opin Chem Biol 2011, 15:174-182; Walters, Adv Drug Deliv Rev 2002, 54:255-271). Hits were profiled in secondary, tertiary and target engagement assays to determine their MOAs and selectivity (Fancher et al., Assay Drug Dev Technol 2016, 14(8):453-477; Fancher et al., Assay Drug Dev Technol 2019, 17(8):364-386).

Five, 124, and 117 hits from the 10K, 50K, and 83K libraries, respectively, exhibited AR-TIF2 PPI inhibitor/disruptor IC₅₀s≤40 μM, passed medicinal chemistry computational filters, exhibited≥90% purity, and were commercially available for resupply. Medicinal chemistry evaluations of ADME/Tox bioavailability properties, chemical tractability, and potential synthetic strategies were used to prioritize hit selections further. Two hits from the 10K ChemDiv PPI library were purchased and subsequently deprioritized due to relatively weak potencies in the AR-TIF2 PPIB assay, IC₅₀s>20 μM for AR-TIF2 PPI formation and >100 μM for disruption. The NCI 83K library hits were deprioritized because they had unfavorable physicochemical properties or due to the presence of reactive functionality such as Michael acceptors (α,β-unsaturated carbonyl groups) or aldehyde moieties that may react covalently and indiscriminately with proteins. Five hits from the 50K ChemBridge diversity library representing three different structural series were prioritized because their IC₅₀s were <20 μM for AR-TIF2 PPI formation and <25 μM for disruption and they had favorable physiochemical properties: Series 1—hydrobenzo-oxazepines, Series 2-thiadiazol-5-piperidine-carboxamides, and Series 3—fluorophenyl-methylindoles (Table A supra). Series 3 compounds were predicted to bind to the allosteric BF-3 site, while Series 1 and 2 compounds bind to a novel BP-1 pocket adjacent to the OSL binding site.

The AR-LBD BF-3 pocket is lined by residues from helices 1, 3, and 9 and is topographically adjacent to but distinct from the AF-2 groove and distal to the OSL site (Buzón ei al., Mol Cell Endocrinol 2012, 384(2):394-402; Estébanez-Perpiñá, et al., PNAS USA 2007, 104(410):10674-10679). Missense mutations in the AR BF-3 pocket are linked to PC, infertility, and/or androgen insensitivity syndromes (Buzón et al.; Solène Grosdidier, Mol Endocrinol 2012, 26(7):1078-1090). BF-3 is a solvent exposed concave hydrophobic pocket that is conserved in steroid NR LBDs, MR, PR, GR, and to some extent ER isoforms.

### Example 2

### Compound Evaluation

Four structurally related analogs of the S1-1 and S2-6 hits were purchased from the ChemBridge parent library, eight analogs of the fluorophenyl-methyl indole hits S3-11 and S3-14, and four analogs of the phenyl-methyl indole hit S3-23. Hits and analogs of the three series were profiled in biochemical and cell based assays to elucidate potential MOA's. The five AF-2 and three AF-1 focused assays utilized to characterize the hits and analogs described here were previously bench marked and validated with seven known AR modulator compounds including; three AR antagonists (flutamide, bicalutamide, and enzalutamide) and one androgen synthesis inhibitor (abiraterone) that are FDA approved ADTs, two investigational molecules (compound #10 and EPI-001) that target the N-terminal domain of AR, and an inhibitor of the Hsp90 molecular chaperone (Fancher et al., Assay Drug Dev Technol. 2019, 17:364-386).

TABLE 4
Hydrobenzo-oxazepines

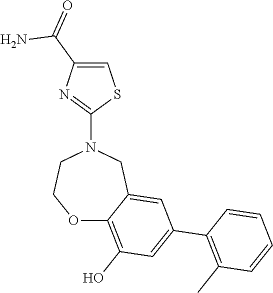 [SMILES: Cc1ccccc1-c1cc(O)c2c(c1)CN(c1nc(C(N)=O)cs1)CCO2]

S1-1 (98648798)
C then D IC50 5.64 μM
D then C IC50 26.6 μM
GI50 > 100 μM

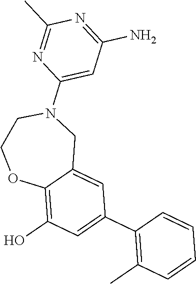 [SMILES: Cc1nc(N)cc(N2CCOc3c(O)cc(-c4ccccc4C)cc3C2)n1]

S1-2 (26996278)
C then D IC50 14.5 μM
D then C IC50 63.0 μM
GI50 51.9 μM

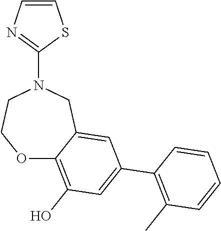 [SMILES: Cc1ccccc1-c1cc(O)c2c(c1)CN(c1nccs1)CCO2]

S1-3 (74301431)
C then D IC50 30.5 μM
D then C IC50 41.8 μM
GI50 > 100 μM

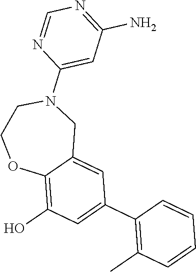 [SMILES: Cc1ccccc1-c1cc(O)c2c(c1)CN(c1cc(N)ncn1)CCO2]

S1-4 (61741848)
C then D IC50 > 100 μM
D then C IC50 > 100 μM
GI50 > 100 μM

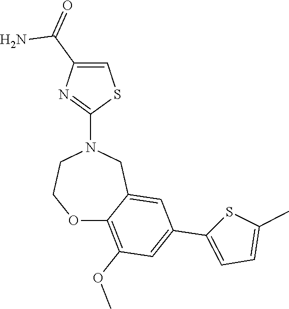 [SMILES: COc1cc(-c2ccc(C)s2)cc2c1OCCN(c1nc(C(N)=O)cs1)C2]

S1-5 (71147047)
C then D IC50 26.2 μM
D then C IC50 > 100 μM
GI50 > 100 μM

Compounds 1-5 were assessed to determine whether each compound could block DHT-induced AR-TIF2 PPI (protein-protein interaction complex) formation and/or disrupt preformed DHT-induced AR-TIF2 PPIs. Additionally, the following activities were evaluated:
inhibition of DHT-induced AR-mediated PSA promoter-driven transcription in C4-2 CRPC cells;
inhibition of DHT-induced AR-LBD PPI interactions with TIF2 and SRC1 coactivators;
inhibition of DHT-induced AR-LBD PPI interactions with TIF2 Box III-LXXLL peptide;
inhibition of H³-DHT binding to AR-LBD;
inhibition of DHT-induced AR-mediated PSA promoter-driven transcription in PC3-AR-FL-GFP cells;
inhibition of constitutive AR-V7-mediated PSA promoter-driven transcription in PC3-AR-V7 GFP cells;
inhibition of constitutive UBE2C promoter-driven transcription in PC3-AR-V7 GFP cells;
inhibition of growth in AR-negative prostate cancer (PCa) cell lines PC3 and DU145; and
inhibition of growth in AR-positive PCa cell lines LNCap, C4-2 (CRPC), and 22Rv1 (CRPC).

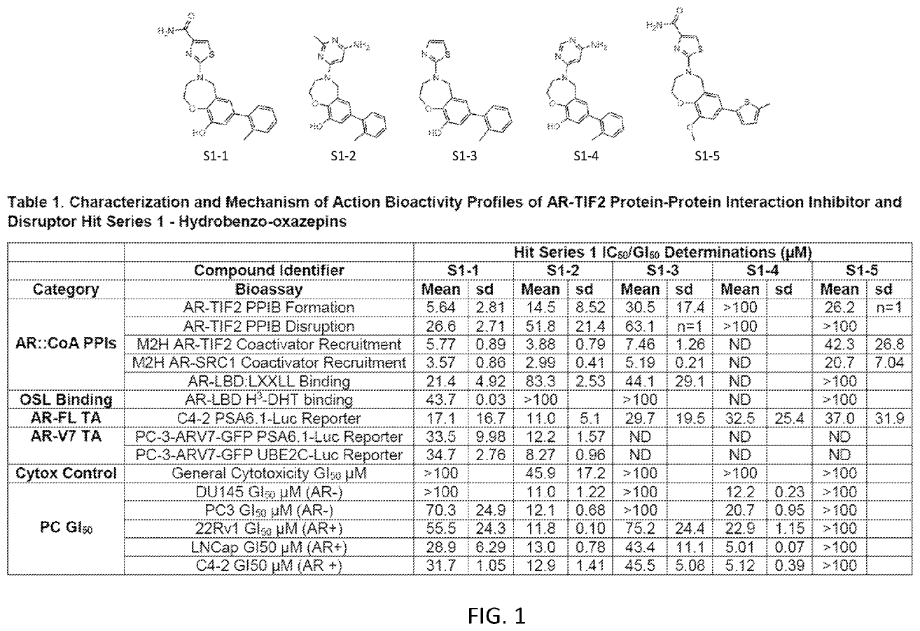

The results are shown in FIG. 1. Compounds 1-3 and 5 inhibited DHT-induced AR-TIF2 PPI formation with IC50s<30 μM. Compound 4 was inactive (IC50>100 μM). Compounds 1-3 disrupted preformed DHT-induced AR-TIF2 PPI complexes with IC50s<63 μM. Compounds 4 and 5 were inactive (IC50>100 μM). Compounds 1-5 inhibited DHT-induced AR-mediated PSA promoter-driven transcription in C4-2 CRPC cells with IC50s<37 μM. Compounds 1-3 inhibited DHT-induced AR-LBD PPI interactions with TIF2 and SRC1 coactivators with IC50s<10 PM. Compound 5 was less potent (IC50s 20-42 μM) and compound 4 was not tested. Compounds 1-3 inhibited DHT-induced AR-LBD PPI interactions with TIF2 Box III-LXXLL peptide with IC50s˜21-83 μM. Compound 5 was inactive (IC50>100 μM) and compound 5 was not tested. Compound 1 inhibited H³-DHT binding to AR-LBD with an IC50˜44 μM. Compounds 2, 3, and 5 were inactive (IC50>100 μM) and compound 4 was not tested. Compounds 1 and 2 inhibited DHT-induced AR-mediated PSA promoter-driven transcription in PC3-AR-FL-GFP cells with IC50s<44 μM. Compounds 3-5 were not tested. Compounds 1 and 2 inhibited constitutive AR-V7-mediated PSA promoter-driven transcription in PC3-AR-V7 GFP cells with IC50s<34 μM. Compounds 3-5 were not tested. Compounds 1 and 2 inhibited constitutive UBE2C promoter-driven transcription in PC3-AR-V7 GFP cells with IC50s<35 μM. Compounds 3-5 were not tested. Compound 5 did not inhibit the growth of any PCa cell lines at ≤100 μM. Compounds 2 and 4 produced GI50s˜5-23 μM range against all 5 PCa cell lines. Compound 1 produced GI50s˜29-70 μM range against all 5 PCa cell lines. Compound 3 produced GI50s˜44-75 μM range against all 5 PCa cell lines. Compounds 1-5 exhibited evidence of better potency against AR+ PCa cell lines.

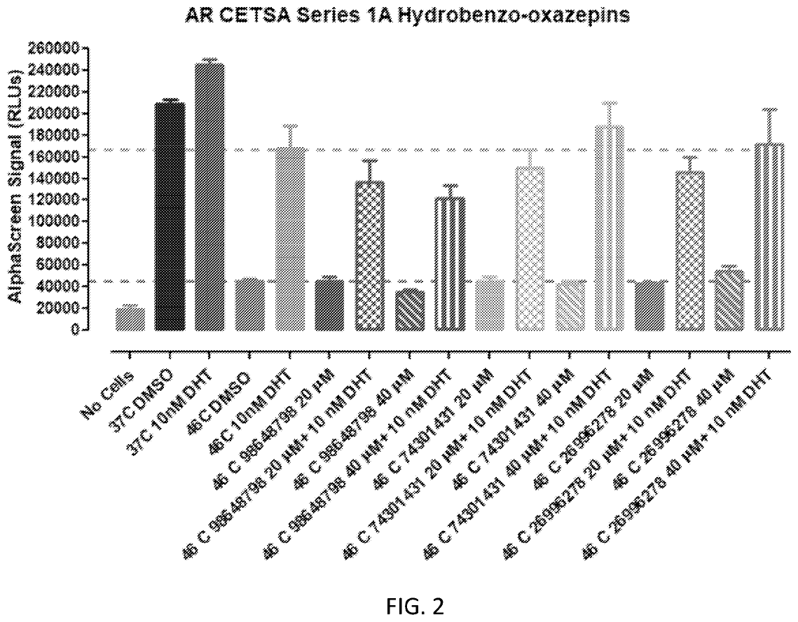

FIG. 2 shows the results of an androgen receptor cellular thermal shift assay (AR-CETSA) of the hydrobenzo-oxazepines. Compounds 1-3 at either 20 or 40 μM did not enhance AR stability at 46° C. (AR western blots and CETSA data). Compounds 1-3 at 20 μM did not enhance DHT-induced AR stability at 46° C. (AR western blots and CETSA data). Compounds 1-3 at 20 μM did not enhance TIF2 thermal stability at 46° C. (TIF2 western blotting data not shown). The results indicate that the hydrobenzo-oxazepines do not appear to directly bind to either AR or TIF2.

TABLE 5
Thiadiazol-5-Piperidine Carboxamides

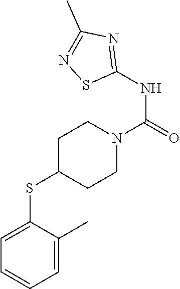 [SMILES: Cc1nsc(NC(=O)N2CCC(Sc3ccccc3C)CC2)n1]

6 (55803564)
C then D IC50 1.06 μM
D then C IC50 5.8 μM
GI50 > 100 μM

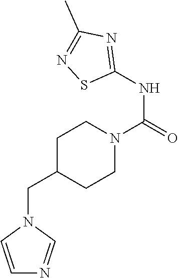 [SMILES: Cc1nsc(NC(=O)N2CCC(Cn3ccnc3)CC2)n1]

7 (26522479)
C then D IC50 > 100 μM
D then C IC50 > 100 μM
GI50 > 100 μM

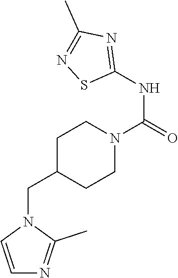 [SMILES: Cc1nsc(NC(=O)N2CCC(Cn3ccnc3C)CC2)n1]

8 (36589964)
C then D IC50 > 100 μM
D then C IC50 > 100 μM
GI50 > 100 μM

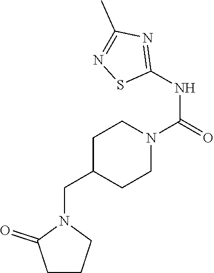 [SMILES: Cc1nsc(NC(=O)N2CCC(CN3CCCC3=O)CC2)n1]

9 (69691085)
C then D IC50 > 100 μM
D then C IC50 > 100 μM
GI50 > 100 μM

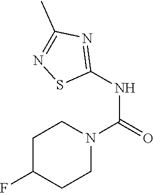 [SMILES: Cc1nsc(NC(=O)N2CCC(F)CC2)n1]

10 (53406864)
C then D IC50 > 100 μM
D then C IC50 > 100 μM
GI50 > 100 μM

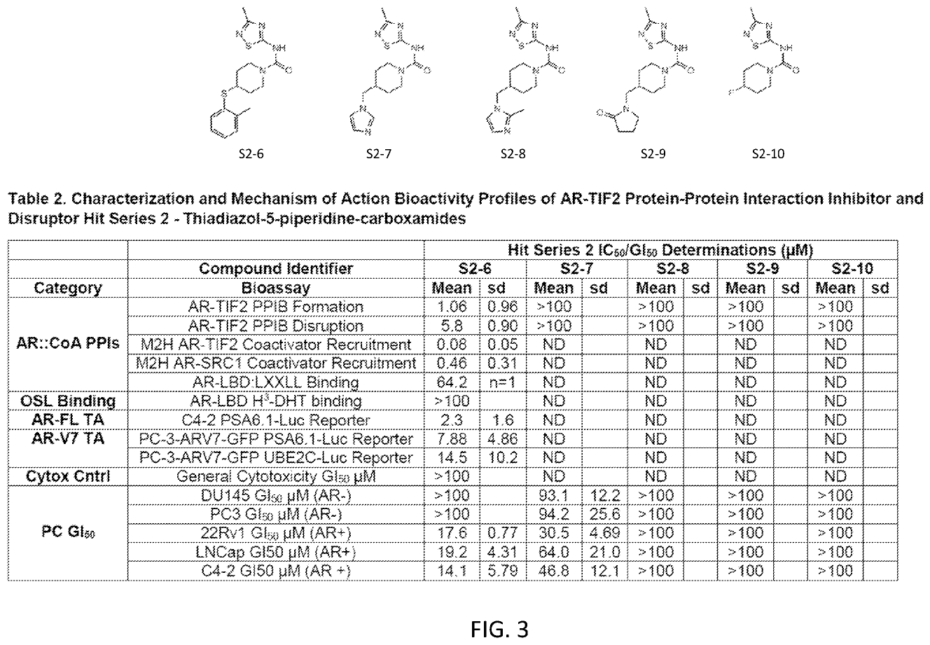

The assays were repeated with thiadiazol-5-piperidine carboxamides. The results are shown in FIG. 3. Compound 6 inhibited DHT-induced AR-TIF2 PPI formation with an IC50˜1 μM. Compounds 7-10 were inactive (IC50>100 μM). Compound 6 disrupted preformed DHT-induced AR-TIF2 PPI complexes with IC50˜6 μM. Compounds 7-10 were inactive (IC50>100 μM). Compound 6 inhibited DHT-induced AR-mediated PSA promoter-driven transcription in C4-2 CRPC cells with an IC50 of 2.3 μM. Compounds 7-10 were inactive (IC50>100 μM). Compound 6 inhibited DHT-induced AR-LBD PPI interactions with TIF2 and SRC1 coactivators with IC50s of 0.1 and 0.4 μM. Compounds 7-10 were not tested. Compound 6 inhibited DHT-induced AR-LBD PPI interactions with TIF2 BOX III-LXXLL peptide with an IC50˜64 μM. Compounds 7-10 were not tested. Compound 6 did not inhibit H³-DHT binding to AR-LBD (IC50>100 μM). Compounds 7-10 were not tested. Compound 6 inhibited DHT-induced AAR-mediated PSA promoter-driven transcription in PC3-AR-FL-GFP cells with an IC50˜28 μM. Compounds 7-10 were not tested. Compound 6 inhibited constitutive AR-V7-mediated PSA promoter-driven transcription in PC3-AR-V7-GFP cells with an IC50˜8 μM. Compounds 7-10 were not tested. Compound 6 inhibited constitutive UBE2C promoter-driven transcription in PC3-AR-V7-GFP cells with an IC50˜15 μM. Compounds 7-10 were not tested. Compound 6 produced GI50s˜14-20 M against the 3 AR+ PCa cell lines and was inactive (IC50>100 PM) against the 2 AR-PCa cell lines. Compound 7 produced GI50s˜30-94 μM range against all 5 PCa cell lines. Compounds 8-10 did not inhibit the growth of any PCa cell lines at ≤100 μM.

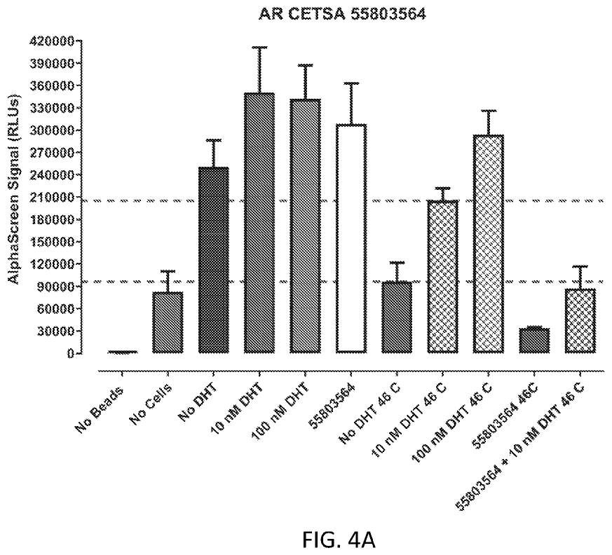

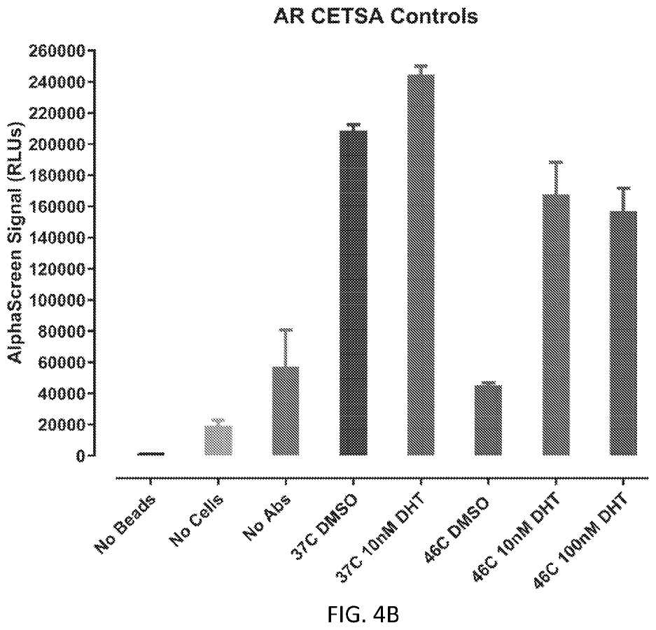

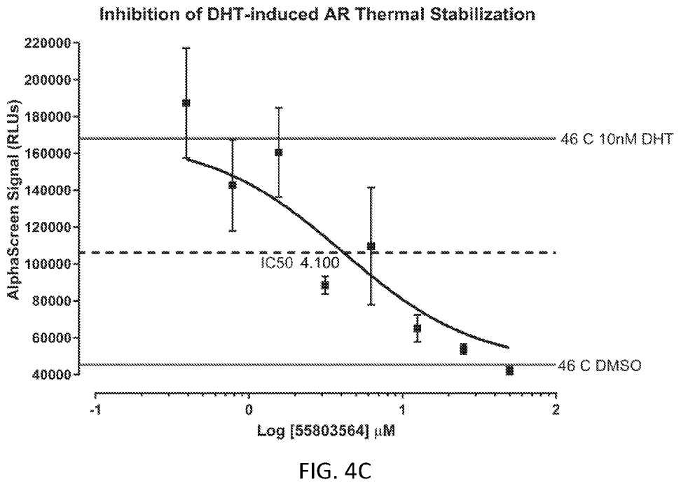

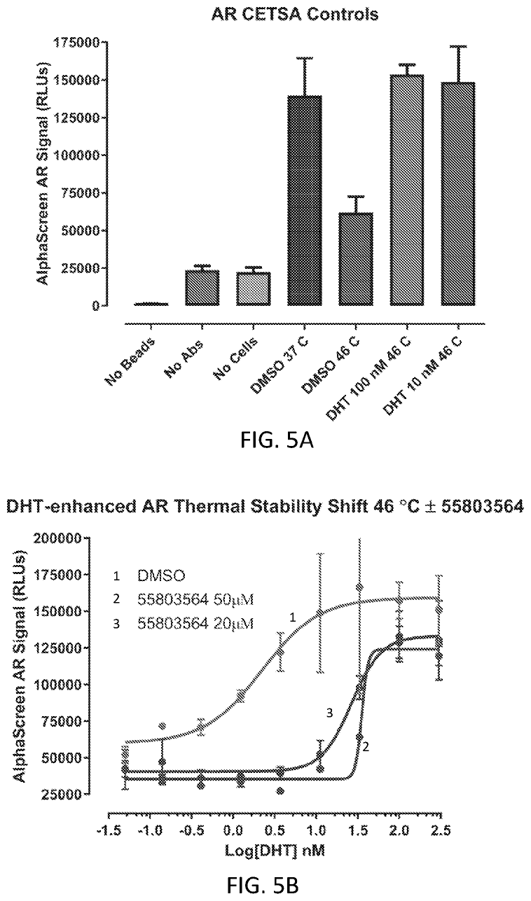

FIGS. 4A-4C show that exposure to 20 μM compound 6 inhibited DHT-induced AR stability at 46° C. Exposure to 20 μM compound 6 did not enhance TIF2 stability at 46° C. (TIF2 western blots—data not shown). Exposure to 20 μM compound 6 did not enhance AR stability at 46° C. (AR western blots and CETSA data; FIGS. 4A-4B). Exposure to 20 μM compound 6 inhibited DHT-induced AR stability at 46° C. (AR western blots and CETSA data; FIGS. 4A-4B). Compound 6 inhibited DHT-induced AR stability at 46° C. with an IC50 of 4.1 μM (AR CETSA data; FIG. 4C). FIGS. 5A-5B show that 20 μM and 50 μM compound 6 inhibited DHT-enhanced AR stability at 46° C.

TABLE 6
Methyl Indoles

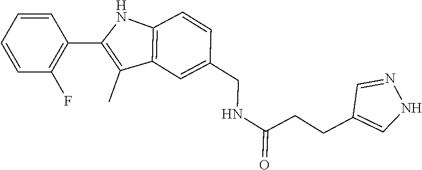 [SMILES: Cc1c(-c2ccccc2F)[nH]c2ccc(CNC(=O)CCc3cn[nH]c3)cc12]

S3-11 (14977726)
C then D IC50 3.25 μM
D then C IC50 24.1 μM
GI50 > 100 μM

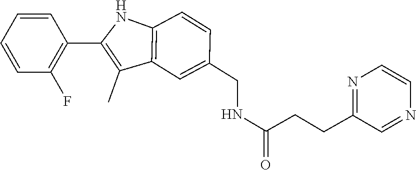 [SMILES: Cc1c(-c2ccccc2F)[nH]c2ccc(CNC(=O)CCc3cnccn3)cc12]

S3-12 (21302587)
C then D IC50 5.13 μM
D then C IC50 24.7 μM
GI50 > 100 μM

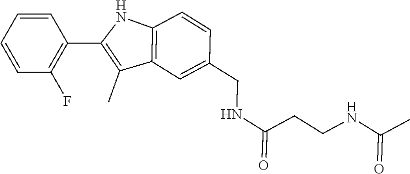 [SMILES: CC(=O)NCCC(=O)NCc1ccc2[nH]c(-c3ccccc3F)c(C)c2c1]

S3-13 (2483178)
C then D IC50 7.98 μM
D then C IC50 37.6 μM
GI50 > 100 μM

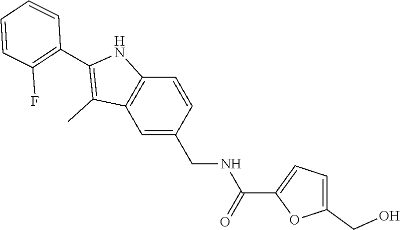 [SMILES: Cc1c(-c2ccccc2F)[nH]c2ccc(CNC(=O)c3ccc(CO)o3)cc12]

S3-14 (36998335)
C then D IC50 2.42 μM
D then C IC50 16.0 μM
GI50 > 100 μM

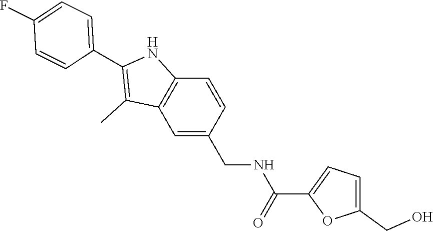 [SMILES: Cc1c(-c2ccc(F)cc2)[nH]c2ccc(CNC(=O)c3ccc(CO)o3)cc12]

S3-15 (60134988)
C then D IC50 6.70 μM
D then C IC50 30.6 μM
GI50 > 100 μM

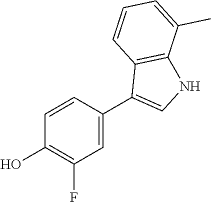 [SMILES: Cc1cccc2c(-c3ccc(O)c(F)c3)c[nH]c12]

S3-17 (63718298)
C then D IC50 3.87 μM
D then C IC50 8.8 μM
GI50 99.0 μM

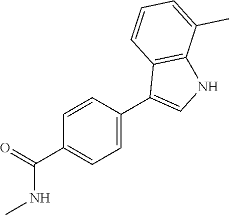 [SMILES: CNC(=O)c1ccc(-c2c[nH]c3c(C)cccc23)cc1]

S3-21 (72508471)
C then D IC50 36.1 μM
D then C IC50 > 100 μM
GI50 > 100 μM

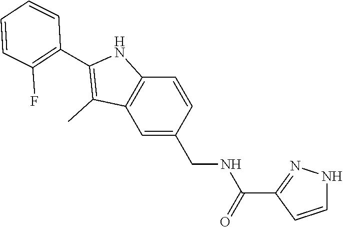 [SMILES: Cc1c(-c2ccccc2F)[nH]c2ccc(CNC(=O)c3cc[nH]n3)cc12]

S3-16 (36023172)
C then D IC50 28.8 μM
D then C IC50 > 100 μM
GI50 > 100 μM

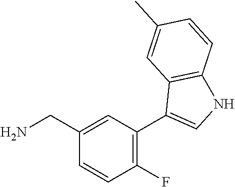 [SMILES: Cc1ccc2[nH]cc(-c3cc(CN)ccc3F)c2c1]

S3-20 (44744397)
C then D IC50 61.5 μM
D then C IC50 71.5 μM
GI50 22.1 μM

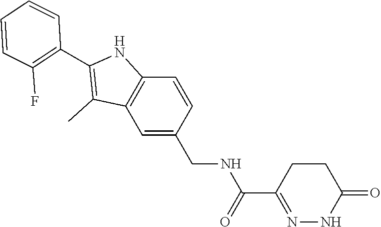 [SMILES: Cc1c(-c2ccccc2F)[nH]c2ccc(CNC(=O)C3=NNC(=O)CC3)cc12]

S3-18 (62182397)
C then D IC50 39.6 μM
D then C IC50 > 100 μM
GI50 > 100 μM

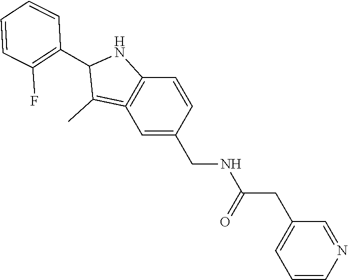

S3-19 (99269016)
C then D IC50 68.5 μM
D then C IC50 > 100 μM
GI50 > 100 μM

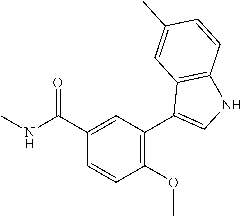 [SMILES: CNC(=O)c1ccc(OC)c(-c2c[nH]c3ccc(C)cc23)c1]

S3-22 (31153802)
C then D IC50 > 100 μM
D then C IC50 > 100 μM
GI50 > 100 μM

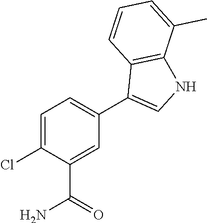 [SMILES: Cc1cccc2c(-c3ccc(Cl)c(C(N)=O)c3)c[nH]c12]

S3-23 (62209680)
C then D IC50 1 μM
D then C IC50 46.2 μM
GI50 > 100 μM

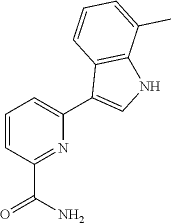 [SMILES: Cc1cccc2c(-c3cccc(C(N)=O)n3)c[nH]c12]

S3-24 (71233599)
C then D IC50 4.10 μM
D then C IC50 26.0 μM
GI50 > 100 μM

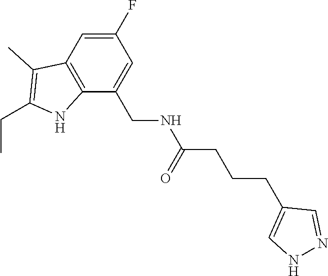 [SMILES: CCc1[nH]c2c(CNC(=O)CCCc3cn[nH]c3)cc(F)cc2c1C]

S3-25 (59706383)
C then D IC50 77.1 μM
D then C IC50 > 100 μM
GI50 > 100 μM

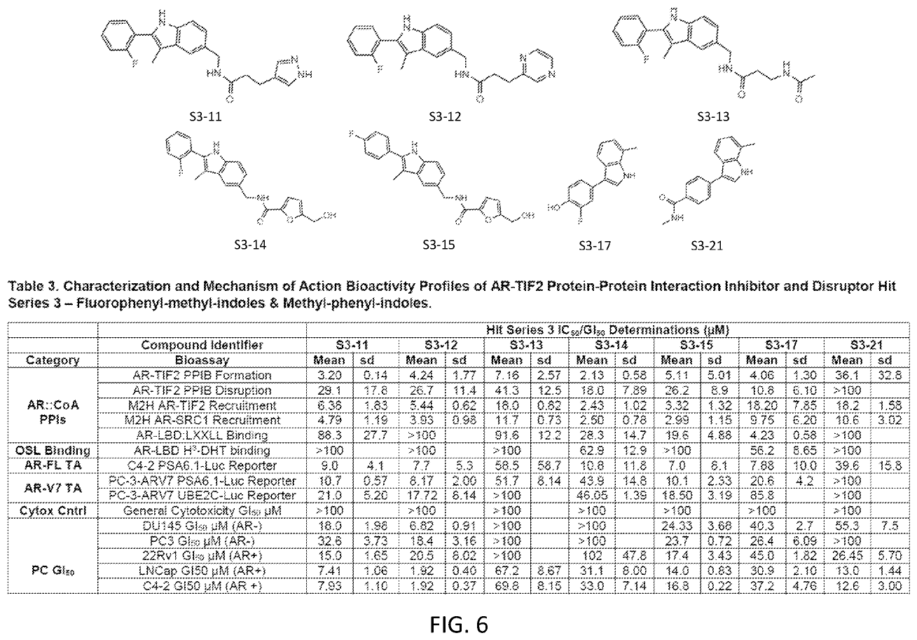

Results for compounds 11-17 are shown in FIG. 6. Compounds 11-16 inhibited DHT-induced AR-TIF2 PPI formation with an IC50s<10 μM. Compound 17 was less potent with an IC50˜36 μM. Compounds 11-16 disrupted preformed DHT-induced AR-TIF2 PPI complexes with IC50s in the 10-41 μM range. Compound 17 was inactive (IC50>100 μM). Compounds 11, 12, and 14-16 inhibited DHT-induced AR-mediated PSA promoter-driven transcription in C4-2 CRPC cells with IC50s in the 7-10 μM range. Compounds 13 and 17 were less potent in the 40-59 M range. Compounds 11, 12, 14, and 15 inhibited DHT-induced AR-LBD PPI interactions with TIF2 and SRC1 coactivators with IC50s<10 μM. Compounds 13, 16, and 17 were less potent with IC50s in the 10-20 μM range. Compounds 14-16 inhibited DHT-induced AR-LBD PPI interactions with TIF2 BOX III-LXXLL peptide with IC50s in the 4-29 μM range. compounds 11 and 13 were less active with IC50s of 88 and 92 μM. Compound 17 was inactive (TC50>100 μM). Compounds 14 and 16 inhibited H³-DHT binding to AR-LBD with IC50s of 63 and 56 μM. Compounds 11-13, 15, and 17 were inactive (IC50>100 μM). Compound 15 inhibited DHT-induced AR-mediated PSA promoter-driven transcription in PC3-AR-FL-GFP cells with an IC50˜10 μM. Compounds 11, 14, and 16 were less active with IC50˜50 μM. Compounds 12, 13, and 17 were inactive (IC50>100 μM). Compounds 11-16 inhibited constitutive AR-V7-mediated PSA promoter-driven transcription in PC3-AR-V7-GFP cells with an IC50s in the 10-50 μM range. Compound 17 was inactive (IC50>100 μM). Compounds 11, 12, and 14-6 inhibited constitutive UBE2C promoter-driven transcription in PC3-AR-V7-GFP cells with an IC50s in the 18-86 μM range. Compounds 13 and 17 were inactive (IC50>100 μM). Compounds 11, 12, 15, and 16 produced GI50s in the 2-45 μM range against all 5 PCa cell lines. Compound 17 produced GT50s˜13-55 μM range against 4 PCa cell lines, with lower IC50s against AR+ cell lines. Compound 14 produced GI50s in the 30-100 μM range against the 3 AR+ PCa cell lines. Compound 13 produced GI50s˜68-70 μM against 2 AR+ PCa cell lines. In general, compounds 11-17 exhibited better potency against AR+ PCa cell lines.

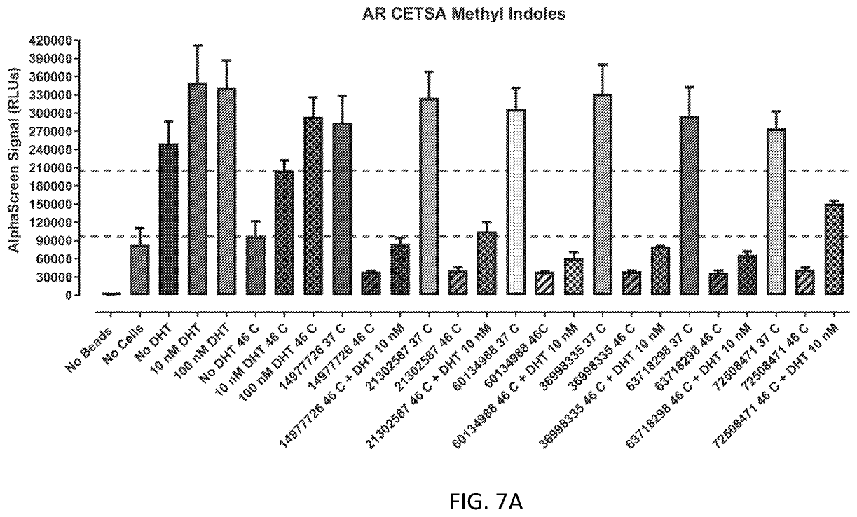

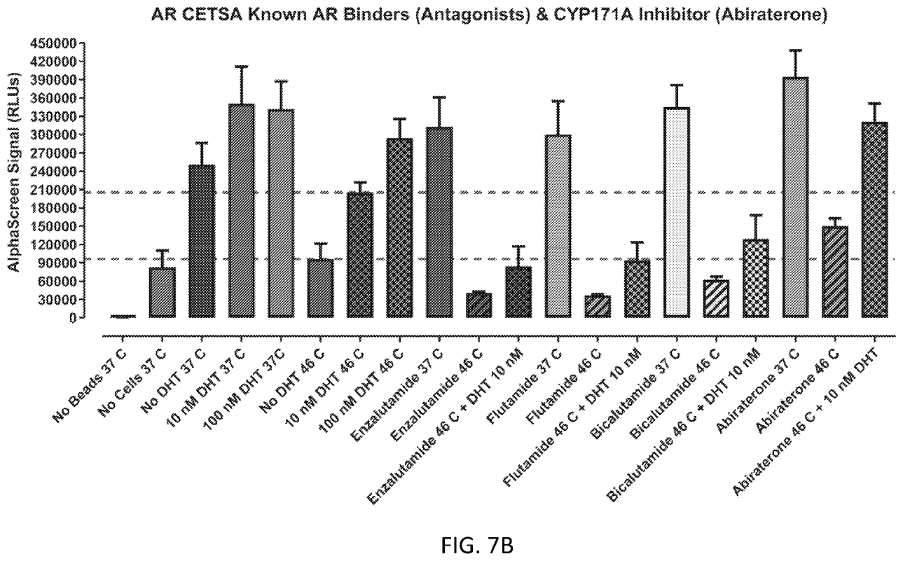

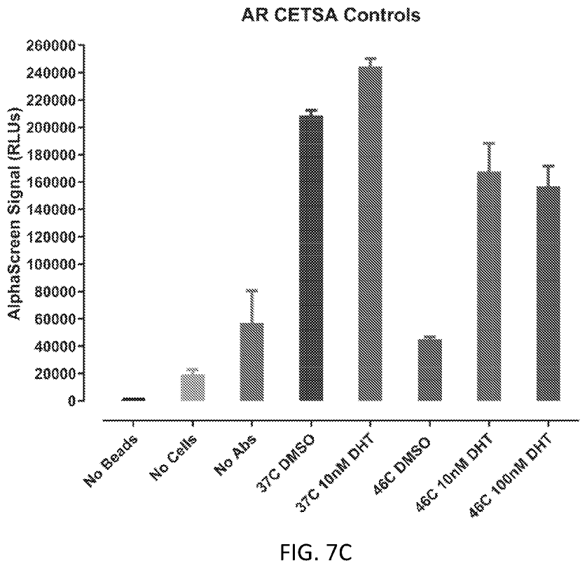

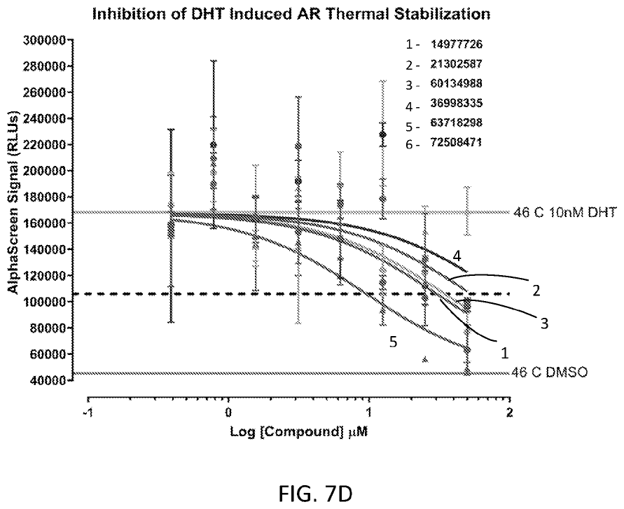

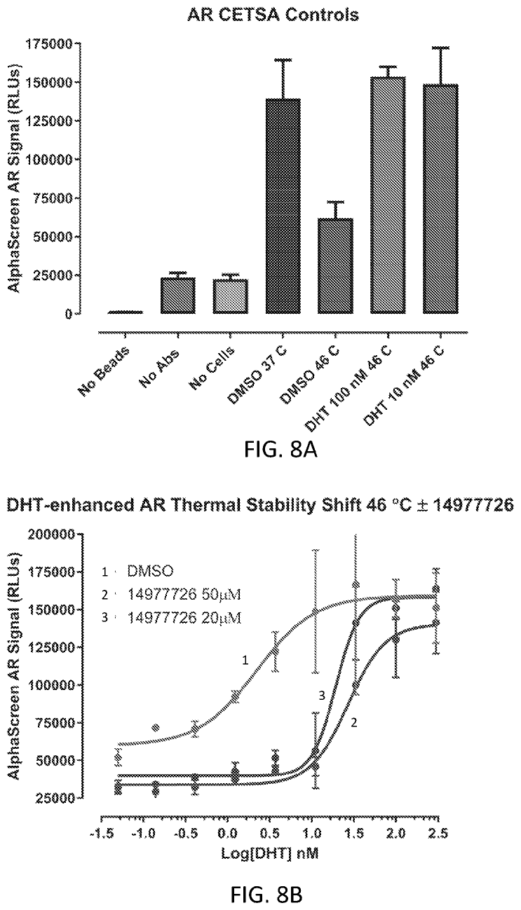

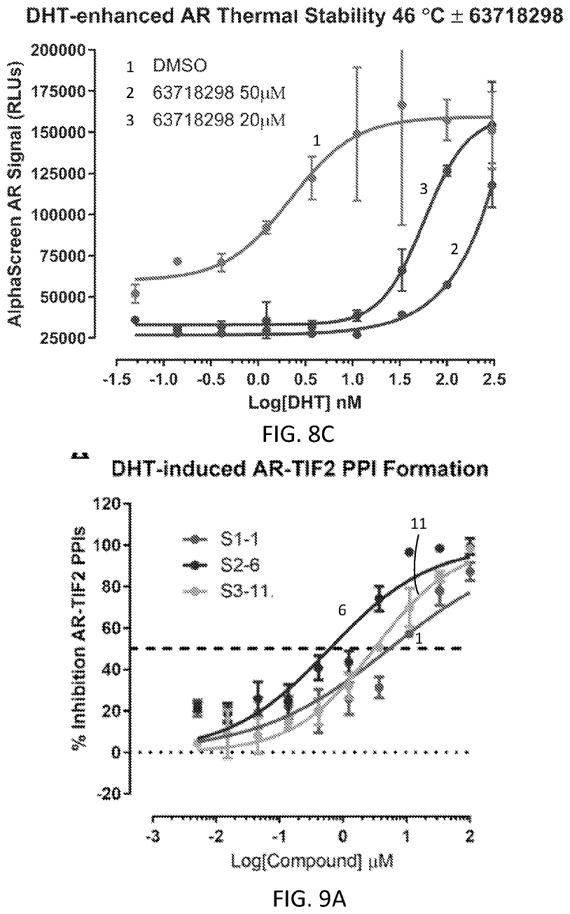

FIGS. 7A-7D show results of AR CETSA assays with the methyl indoles. Exposure of C4-2 cells to AR control and methyl indole compounds for 2 h at 20 μM at 37° C. produced AR signals consistent with the levels observed in untreated cells or cells exposed to 10 or 100 nM DHT (FIG. 7A). Exposure of C4-2 cells to AR antagonists (enzalutamide, flutamide, and bicalutamide) and methyl indole compounds for 2 h at 20 μM did not enhance the thermal stability of AR incubated at 46° C. for 5 min (FIG. 7B). The CYP171A inhibitor abiraterone appeared to enhance the thermal stability of AR incubated at 46° C. for 5 min (FIG. 7B). AR antagonists and methyl indole compound exposure may have further destabilized AR at 46° C. for 5 min. Exposure of C4-2 cells to AR antagonists and methyl indole compounds for 2 h at 20 μM inhibited the ability of 10 nM DHT to enhance the thermal stability of AR incubated at 46° C. for 5 min (FIG. 7B). Abiraterone appeared to enhance the DHT-induced thermal stability of AR incubated at 46° C. for 5 min (FIG. 7B). Exposure of C4-2 cells to the methyl indoles, except compound 17, for 2 h inhibited the ability of 10 nM DHT to enhance the thermal stability of AR incubated at 46° C. for 5 min in a concentration-dependent manner (FIG. 7D). The IC50s were as follows: compound 11-29.91 μM, compound 12-52.57 μM, compound 14-9.459, compound 15-86.09 μM, compound 16-9.459 μM. Exposure to 20 μM methyl-indole hits did not enhance TIF2 stability at 46° C. (TIF2 western blots—data not shown). FIGS. 8A-8C show that 20 μM and 50 μM compound 11 (FIG. 8B) and compound 16 (FIG. 8C) inhibited DHT-enhanced AR stability at 46° C.

The bioactivity profiles of the most active compounds in each series, compounds S1-1, S2-6, and S3-11, are shown in Table 7.

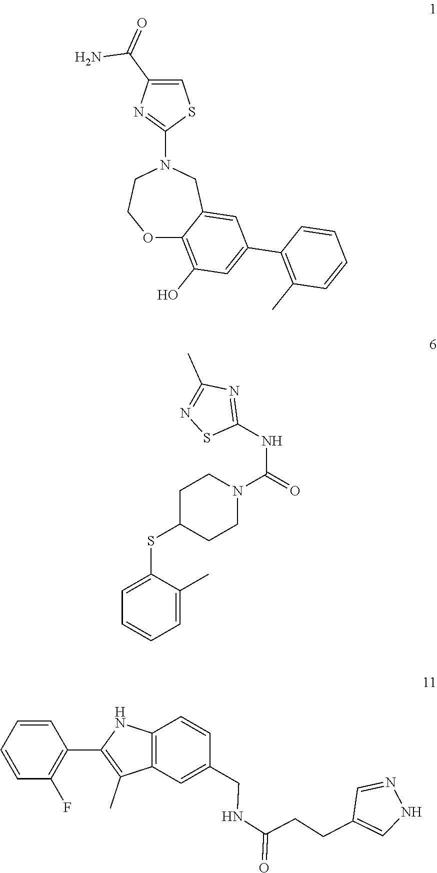 [SMILES: Cc1ccccc1-c1cc(O)c2c(c1)CN(c1nc(C(N)=O)cs1)CCO2 ; Cc1nsc(NC(=O)N2CCC(Sc3ccccc3C)CC2)n1 ; Cc1c(-c2ccccc2F)[nH]c2ccc(CNC(=O)CCc3cn[nH]c3)cc12]

TABLE 7

| | Bioactivity Profiles | | |
|---|---|---|---|
| | Compound and IC₅₀ (μM) | | |
| Bioassay | 1 | 6 | 11 |
| Inhibition DHT-induced AR-TIF2 PPI Formation | 5.38 | 0.62 | 3.11 |
| Disruption of DHT-induced AR-TIF2 PPIs | 24.8 | 5.64 | 26.1 |
| Inhibition DHT-induced AR-TIF2 mammalian 2 hybrid assay | 0.91 | 0.05 | 0.89 |
| Inhibition DHT-induced AR-SRC1 mammalian 2 hybrid assay | 0.38 | 0.29 | 0.60 |
| Inhibition DHT-induced TIF2 NR Box 3 LXXLL peptide | 12.0 | 49.5 | 45.2 |
| binding to AR-LBD | | | |
| Inhibition H³-DHT ligand binding to AR-LBD | 26.7 | >100 | >100 |
| Inhibition of DHT-induced full length AR PSA reporter | 5.27 | 0.51 | 4.21 |
| Inhibition AR-V7 PSA-reporter | 10.5 | 3.82 | 3.13 |
| Inhibition AR-V7 UBE2C-reporter | 21.2 | 10.9 | 12.0 |
| AR negative DU-145 GI₅₀ | 60.5 | >100 | 15.9 |
| AR negative PC-3 GI₅₀ | 61.1 | >100 | 28.4 |
| AR positive 22Rv1 GI₅₀ | 42.5 | 17.9 | 13.2 |
| AR positive LnCaP GI₅₀ | 24.4 | 15.6 | 6.35 |
| AR positive C4-2 GI₅₀ | 30.7 | 8.66 | 6.92 |

As shown in Table 7 and the figures, compounds 1, 6, and 11 inhibited DHT-induced AR-TIF2 PPI formation with IC₅₀s in the 0.62 to 5.4 μM range. Importantly, they also disrupted preformed AR-TIF2 PPI complexes, albeit with 5- to 8-fold higher IC50s. Established biochemical and cell based assays were used to characterize hits and determine their modes of action (MOAs) (Fancher et al., Assay Drug Dev Technol. 2016, 14 (8): 453-477; Fancher et al., Assay Drug Dev Technol. 2019, 17 (8): 364-386). Mammalian 2-Hybrid (M2H) assays are the gold standard for assessing NR-co-regulator interactions that modulate TA (Lievens et al., Trends Biochem Sci 2009, 34(11):579-88; Mendonca et al., Methods Mol Biol 2013, 977:323-38; Stynen et al., Microbiol Mol Biol Rev 2012, 76(2):331-82; Ravasi et al., Cell 2010, 140(5):744-52). In M2H PPI assays between AR-LBD and TIF2 or SRC-1 (Fancher et al., Assay Drug Dev Technol 2019, 17(8):364-368), the hits exhibited IC₅₀s in the 50-600 nM range. The AR-LBD AF-2 surface interacts with CoAs containing LXXLL binding motifs to regulate androgen dependent TA (Bevan et al., Mol Cell Biol 1999, 19(12):8383-92; Dubbink et al., Mol Endocrinol 2004, 18(9):2132-50; He et al., J Biol Chem 2002, 277(12):10226-35; Dubbink et al., Mol Endocrinol 2006, 20(8):1742-55). All three hits inhibited the DHT-induced TIF2 box 3 LXXLL-peptide binding to AR-LBD AlphaScreen® assay (Fancher et al., Assay Drug Dev Technol 2019, 17(8):364-368), with IC₅₀s in the 12 μM (compound 1) to 50 μM (compounds S2-6 and S3-11) range. None of the hits bind to the AF-2 groove and these data are consistent with an allosteric mediated conformation shift reducing CoA binding to AF-2 (Buzón et al., Mol Cell Endocrinol 2012, 384(2):394-402; Estébanez-Perpiñá et al., PNAS USA 2007, 104(41):10674-10679; Solène Grosdidier et al., Mol Endocrinol 2012, 26(7):1078-1090; Lallous et al., Mol Cancer Ther 2016, 15(12):2936-2945). The hits inhibited H³-DHT binding to AR-LBD in a concentration dependent manner (Fancher et al., Assay Drug Dev Technol. 2016, 14(8):453-477; Fancher et al., Assay Drug Dev Technol. 2019, 17(8):364-386), but only compound 1 produced a calculable IC₅₀ (˜27 μM). The hits inhibited DHT-induced PSA-6.1-Luc activity with IC₅₀s in the 0.5 to 5 μM range in a PSA-6.1-luciferase reporter controlled by a PSA promoter fragment with >3 AREs that provides a readout of AR-TA controlled by full length AR in C4-2 CRPC cells (Id.). Ubiquitin-conjugating enzyme E2C (UBE2C) is a specific target gene of AR splice variants (Hu et al., Cancer Res 2012, 72(14):3457-62; Cao et al., Oncotarget 2014, 5(6):1646-56; Xu et al., Cancer Res 2015, 75(17):3663-71). The UBE2C luciferase reporter is driven by 3 AR-V7-specific promoter element repeats from the UBE2C gene (Xu et al., Cancer Res 2015, 75(17):3663-71). To determine if hits block ligand-independent AF-1 directed splice variant TA, the PSA6-6.1-Luc and UBE2C-Luc reporters were transfected into PC3-AR-V7-EGFP cells (Fancher et al., Assay Drug Dev Technol. 2019, 17(8):364-386). The hits inhibited AR-V7 driven PSA6.1-Luc and UBE2C-Luc reporter assays with IC₅₀s in the 3 μM to 10-20 μM range. The inhibition of constitutive TA by AR NTD splice variants lacking an LBD was surprising even though splice variants like AR-V7 also require CoAs like SRC-1 and TIF2 to activate TA (Beavan et al., Mol Cell Biol 1999, 19(12):8383-92; Callewaert et al., Cancer Res 2006, 66(1):543-53; Christiaens et al., J Biol Chem 2002, 277(51):49230-7; Lavery et al., Biochem J 2005, 391 (Pt 3):449-64; Ueda et al., J Biol Chem 2002, 277(41):38087-94). One potential MOA is that the hits may disrupt AR-V7s interactions with full length AR (Xu et al., Cancer Res 2015, 75(17):3663-71, Lv et al., J Clin Invest 2021, 131). Compounds that inhibit CoA recruitment and AR-TA by both AF-2 and AF-1 surfaces would be desirable novel drug candidates for development into CRPC therapies. In growth inhibition assays, the hits exhibited differential cytotoxicity in AR positive PC cell lines. Five Series 1 and Series 2, and fifteen Series 3 structurally related HCS hits or purchased analogs were profiled in these bioassays and the medicinal chemistry evaluation of nascent SARs, drug-like properties, and synthetic/SAR tractability led to their prioritization.

AR-TIF2 protein-protein interaction biosensor inhibition/disruption: The three representative hits from the three series S1-1, 52-6, and S3-11 inhibited DHT-induced AR-TIF2 PPI formation with IC₅₀s in the 1.06 to 5.64 μM range (Tables 4-6, FIGS. 1, 3, 6, 9A). They also disrupted preformed AR-TIF2 PPI complexes, albeit with 5- to 8-fold higher IC₅₀s (FIGS. 1, 3, 6, 9B). Substitution of a 2-methyltiophene group for the toluene group at the R2 position of the S1-1 hydro benzo ring in S1-5 produced a 4-fold reduction in relative potency in both AR-TIF2 PPIB assay formats (Table 4, FIG. 1). For the S1-2, S1-3, and 51-4 analogs, changing the thiazole-4-carboxamide group at the R1 position of the oxazepane ring while maintaining a toluene group at R2 on the hydro benzo ring also reduced their relative potencies in both AR-TIF2 PPIB assay formats (Table 4, FIG. 1). Changing the thiazole-4-carboxamide group at the R1 position of S1-1 to a pyrimidin-4-amine group in S1-4 did not achieve ≥50% inhibition at ≤100 μM, while the 2-methylpyrimidin-4-amine substitution in S1-2 led to only ˜2-fold loss in potency (Table 4, FIG. 1). The four analogs S2-7, S2-8, 52-9, and S2-10 of the thiadiazol-5-piperidine-carboxamide hit S2-6 that have different substituents than the o-tolylthio group at the single R position of the piperidine ring were inactive at ≤100 μM in both AR-TIF2 PPI assay formats (Table 5, FIG. 3). For analogs of the fluorophenyl-methyl indole hits S3-11 and S3-14, the position of the fluorine in S3-15 and S3-17 was different from the hits and other analogs (Table 6). However, most of the analogs differed in the substituents at the R position of the methyl indole region (Table 6). Altering the position of the fluorine in the phenyl ring between the S3-14 hit and S3-15 analog reduced the relative potency in both AR-TIF2 PPIB assay formats by ˜2-fold (Table 6, FIG. 6). Changing the groups at the R position of the methyl indole region of the analogs was reasonably well tolerated, except in S3-18, S3-19, and S3-20 (Table 6). Altering the phenyl ring substitutions and their positions in the analogs of the S3-23 phenyl-methyl indole hit modulated their relative potencies in both AR-TIF2 PPI inhibitor/disruptor assay formats (Table 6). Cells were exposed to compounds at the indicated concentrations for only 4.5 h in the AR-TIF2 PPIB assays. None of the hits and analogs reduced the number of Hoechst stained nuclei below DMSO controls, indicating that cell loss and/or acute cytotoxicity did not contribute to their AR-TIF2 PPI inhibitor/disruptor IC₅₀s. The exploration of the structure activity relationships (SAR) for the three chemical series was limited by the availability of analogs for purchase (Tables 4-6, FIGS. 1, 3, 6, 9A-9L), and future studies will apply medicinal chemistry directed synthesis to expand the nascent SARs.

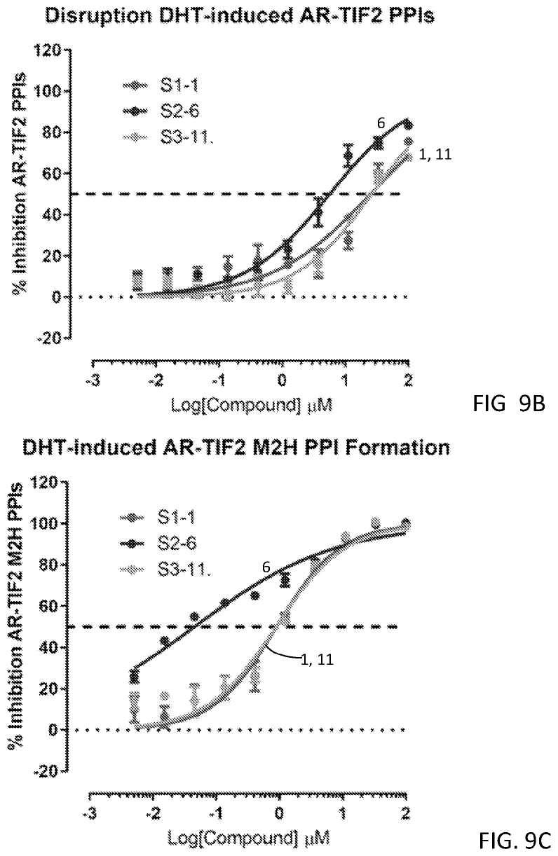

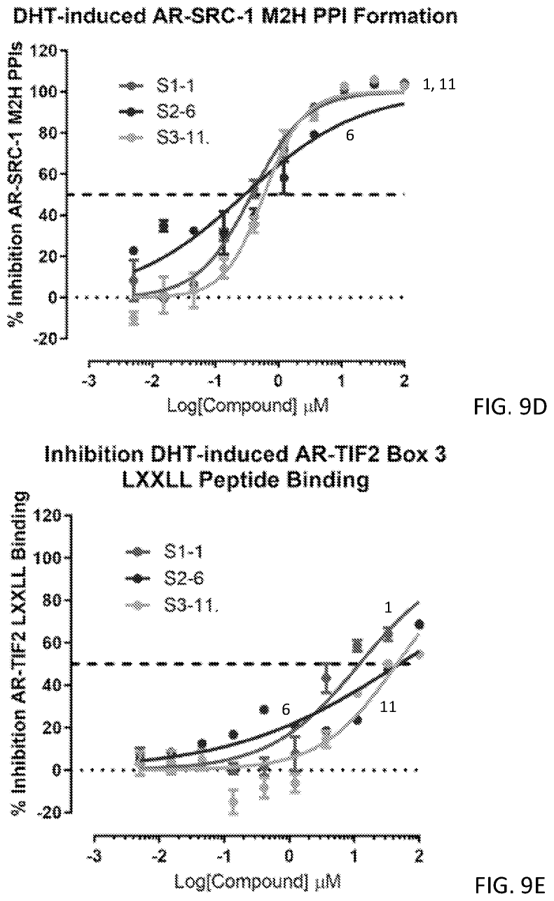

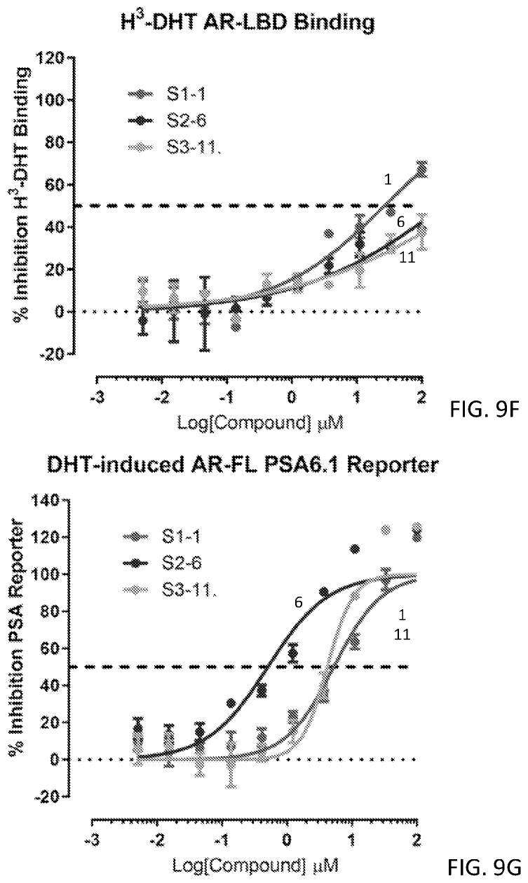

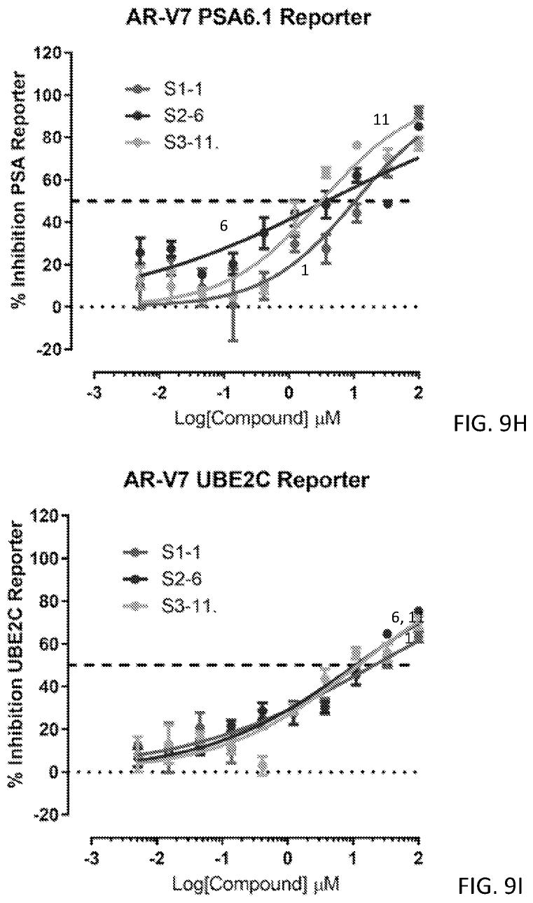

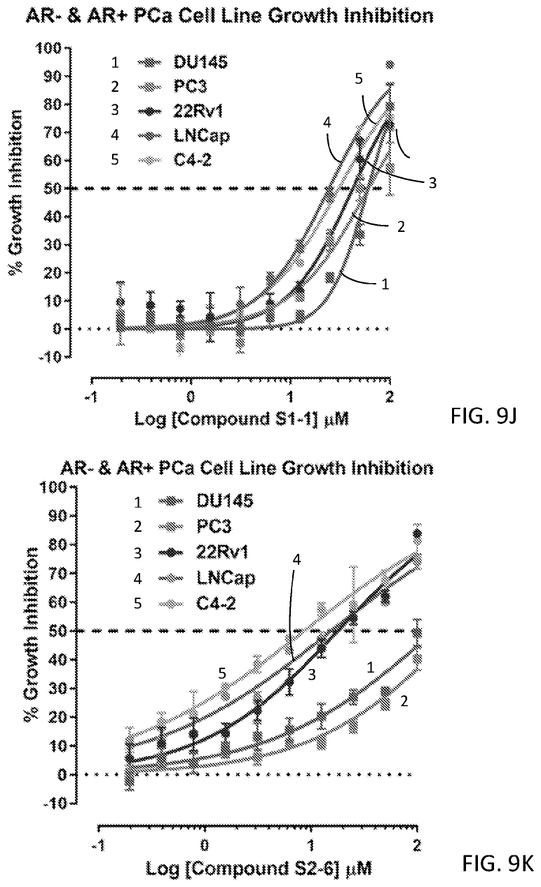

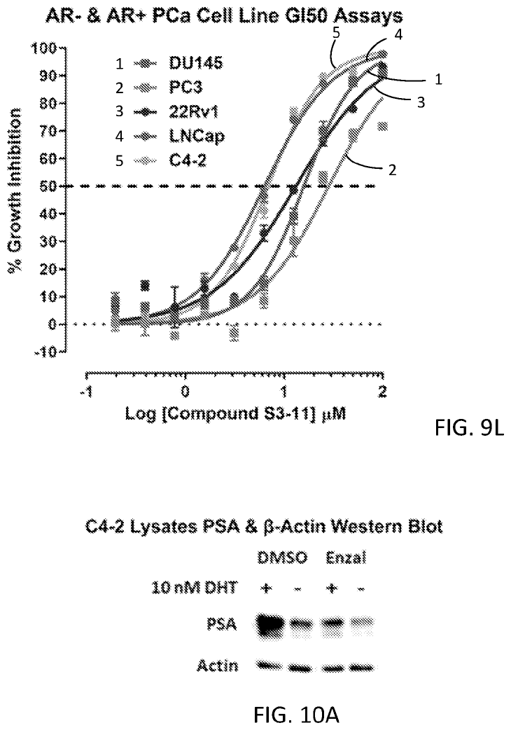

Inhibition of AR—p160 steroid receptor coactivator mammalian 2-hybrid transcriptional activation: For >14 years, mammalian 2-hybrid (M2H) assays have been the gold standard for measuring NR interactions with co-regulators that modulate TA (Lievens et al., Trends Biochem Sci 2009, 34679-88; Mendonca et al., Methods Mol Biol 2013, 977:323-38; Stynen et al., Microbiol Mol Biol Rev 2012, 76:331-82; Ravasi et al., Cell 2010, 140:744-52). Assays were performed to determine whether AR-TIF2 PPI inhibitor/disruptor hits and analogs from the three chemical series would block AR-TIF2 interactions and TA in orthogonal M2H assays, and if they might exhibit selectivity for TIF2 (SRC-2) over the SRC-1 p160 CoA family member. In DHT-activated M2H assays between AR-LBD and either TIF2 or SRC-1 (Fancher et al., Assay Drug Dev Technol. 2019, 17:364-386), the S1-1 hit produced IC₅₀s in the low μM (1 to 10 μM) range for both CoAs consistent with its biosensor IC₅₀ for AR-TIF2 PPI formation, but˜5-fold more potent than its IC₅₀ for AR-TIF2 PPI disruption (FIGS. 1, 9C, 9D). S1-2 and S1-3 analogs inhibited M2H assays with IC₅₀s in the low μM range, 51-5 was less potent with IC₅₀s in the mid μM (10-100 μM) range, and S1-4 was inactive at ≤100 μM (FIG. 1). In TIF2 and SRC1 M2H assays, cells were exposed to compounds at the indicated concentrations for 27 h. The S1-2 analog was the only compound that was active in cytotoxicity counter screens, producing an IC₅₀ of 45.6 μM, >10-fold higher than its corresponding IC₅₀s for the TIF2 and SRC1 M2H assays respectively. The S2-6 hit produced sub-μM (<1 μM) potencies in the TIF2 and SRC-1 M2H assays respectively, ˜10-fold less than it's corresponding AR-TIF2 biosensor IC₅₀s (FIGS. 3, 9C, 9D). S2-6 was the only hit that exhibited evidence of CoA selectivity with ˜5-fold lower IC₅₀ for TIF2 than SRC1 (FIGS. 3, 9C, 9D). S2-6 analogs that were inactive in AR-TIF2 biosensor assays were not tested in M2H assays. The S3-11 and S3-14 hits produced IC₅₀s in the low μM range in both M2H assays consistent with their IC₅₀s for inhibition of DHT-induced AR-TIF2 PPI formation, and >5-fold less potent than their IC₅₀s for AR-TIF2 PPI disruption (FIGS. 6, 9C, 9D). The S3-12 and S3-15 analogs exhibited comparable activity in the M2H assays with IC₅₀s in the low μM range, while S3-13 and S3-17 were less active with IC₅₀s in the mid μM range (FIG. 6). The S3-21 analog was also less active in the M2H assays with IC₅₀s in the mid μM range (FIG. 6). Overall, hits and analogs that inhibited and/or disrupted AR-TIF2 PPIs in biosensor assays also blocked AR TA responses in orthogonal M2H assays between AR-LBD and both p160 CoAs (FIGS. 1, 3, 6, 9C, 9D).

Inhibition of DHT-induced TIF2 box 3 LXXLL-peptide binding to AR-LBD: The LXXLL motifs of p160/SRc CoAs mediate binding to the AF-2 surface of AR resulting in activation of gene transcription (Bevan, et al., Mol Cell Biol 1999, 19:8383-92; Dubbink et al., Mol Endrocrinol 2004, 18:2132-50; He et al., J Biol Chem 2002, 277:10226-35; Dubbink et al., Mol Endocrinol 2006, 20:1742-55). In an AlphaScreen® assay that measures DHT-induced binding of a TIF2 box 3 LXXLL-peptide to recombinant AR-LBD (Fancher et al., Assay Drug Dev Technol. 2019, 17:364-38), representative hits from the three chemical series (S1-1, S2-6, and S3-11) produced IC₅₀s in the mid μM range (FIGS. 1, 3, 6, 9E). S1-2 and S1-3 analogs also inhibited DHT-induced TIF2 LXXLL-peptide binding to AR-LBD with IC₅₀s in the mid 44 μM range, while S1-5 was inactive at ≤100 μM. S1-4 and S2-6 analogs were not tested in the TIF2 LXXLL-peptide binding assay because they were inactive in both AR-TIF2 PPIB formats (FIGS. 1, 3). The S3 hits (S3-11 and S3-14) and analogs (S3-13 and S3-15) produced mid μM IC₅₀s in the TIF2 LXXLL-peptide AR-LBD binding assay, while the S3-17 analog produced a low μM IC₅₀ and both S3-12 and S3-21 analogs were inactive at ≤100 μM (FIGS. 6, 9E). Five compounds with IC₅₀s˜50 μM were identified in an HTS campaign of 55,000 compounds performed in a fluorescence polarization assay that measured the binding of a 15 amino acid LXXLL peptide from TIF2 to the AR-LBD; flufenamic acid, tolefenamic acid, meclofenamic acid, tri-iodothyronine, and triiodothyroacetic acid (Estébanez-Perpiñá, et al., PNAS USA 2007, 104:10674-10679). X-ray diffraction analysis of AR-LBD crystal soaking experiments in the presence of DHT indicated that the five compounds bind in the BF-3 pocket of AR to allosterically remodel the adjacent AF-2 surface thereby weakening its ability to engage in contacts with CoAs (Estébanez-Perpiñá, et al., PNAS USA 2007, 104:10674-10679; Solène Grosdidier et al., Mol Endocrinol. 2012, 26:1078-1090). The relatively high mid μM IC₅₀s in the AR-LBD TIF2 LXXLL-peptide binding assay (FIGS. 1, 3, 6, 9E) suggests that direct antagonism of LXXLL motif binding to the AF-2 surface of AR may not be the primary MOA of the AR-TIF2 inhibitor/disruptor hits and analogs. However, they may be allosteric modulators (AM) capable of inducing AR conformational changes that diminish CoA binding (Estébanez-Perpiñá, et al., PNAS USA 2007, 104:10674-10679; Solène Grosdidier et al., Mol Endocrinol. 2012, 26:1078-1090; Buzón et al., Mol Cell Endocrinol. 2012, 384:394-402; Lallous et al., Mol Cancer Ther. 2016, 15:2936-2945).

Inhibition of H³-DHT binding to AR-LBD: It has previously been shown that AR antagonists and steroid NR ligands that competitively displace H³-DHT binding to recombinant AR-LBD inhibit both formats of the AR-TIF2 PPIB assay and TA reporter assays driven by full length AR and/or AR-V7 splice variants (Fancher et al., Assay Drug Dev Technol. 2016, 14:453-477; Fancher et al., Assay Drug Dev Technol. 2019, 17:364-386). In competitive H³-DHT displacement binding assays to recombinant AR-LBD, six AR antagonists and seven steroid NR ligands produced IC₅₀s in the sub to mid μM range (Fancher et al., Assay Drug Dev Technol. 2016, 14:453-477; Fancher et al., Assay Drug Dev Technol. 2019, 17:364-386). Although representative hits from the three series displayed evidence of concentration dependent inhibition of H³-DHT binding to AR-LBD, only S1-1 produced a calculable IC₅₀ (˜44 μM) (FIGS. 1, 3, 6, 9F). The S1-2, S1-3, and S1-5 analogs did not achieve ≥50% inhibition of H³-DHT binding at ≤100 μM, and S1-4 was not tested (FIG. 1). S2-6 analogs inactive in the AR-TIF2 biosensor assays were also not tested in the H³-DHT AR-LBD binding assay. The S3-14 hit and S3-17 analog produced mid μM IC₅₀s in the H³-DHT binding assay, while the S3-12, S3-13, S3-15, and S3-21 analogs were inactive at ≤100 μM (FIG. 6). The original intent was to use the AR-LBD H³-DHT binding assay to identify and deprioritize AR antagonist hits (Fancher et al., Assay Drug Dev Technol. 2016, 14:453-477; Fancher et al., Assay Drug Dev Technol. 2019, 17:364-386), in part because of the many approved PC drugs that share this MOA, but also because drug resistance inevitably limits the duration of anti-androgen efficacy against CRPC (Harris et al., Nat Clin Pract Urol 2009, 6:76-85; Karantanos et al., Oncogene 2013, 32:5501-11; Gregory et al., Cancer Res 2001, 61:4315-9). Since most of the AR-TIF2 inhibitor/disruptor hits and analogs failed to achieve ≥50% inhibition at ≤100 μM in the H³-DHT AR-LBD binding assay, it is unlikely that direct antagonism of DHT binding to AR is the MOA of these compounds. However, since AM induced conformational changes may also reduce orthosteric ligand binding (Conn et al., Nat Rev Drug Discov 2009, 8:41-54; Bingson Han et al., ACS Med Chem Lett 2020, 11:1810-1819; Zhang et al., J Med Chem. 2020, 63:15258-15278), compounds that exhibited weak or partial inhibition of H³-DHT binding to AR-LBD were not deprioritized.

Inhibition of full length AR transcriptional activation: To determine if AR-TIF2 PPI inhibitor/disruptor hits and analogs blocked DHT-induced full length AR directed TA, a luciferase reporter assay controlled by the PSA promoter (PSA-6.1-Luc) containing ≥3 AREs was conducted in C4-2 CRPC cells (Fancher et al., Assay Drug Dev Technol. 2016, 14:453-477; Fancher et al., Assay Drug Dev Technol. 2019, 17:364-386). Representative hits inhibited DHT-induced AR PSA-Luc reporter activity with IC₅₀s in the 2 to 17 μM range (FIGS. 1, 3, 6, 9G). All four S1 analogs inhibited DHT-induced PSA-Luc reporter activity with IC50s in the mid μM range, comparable to the S1-1 hit (FIG. 1). The S2-6 hit produced an IC₅₀ of 2 μM in the PSA-Luc reporter assay, but the 4 analogs that were inactive in the AR-TIF2 biosensor assays were not tested (FIG. 3). The S3-11 and S3-14 hits produced IC₅₀s in the low μM range in the PSA-Luc reporter assay, comparable to the low μM IC50s of the S3-12, S3-15, and S3-17 analogs (FIG. 6). The S3-14 and S3-21 analogs were less potent in the PSA-Luc reporter assay with mid μM IC₅₀s (FIG. 6). Cells were exposed to the indicated compound concentrations for 24 h in the PSA-Luc reporter assay. Only the S1-2 analog exhibited activity in the cytotoxicity counter screen, producing an IC₅₀ of 45.6 μM, >4-fold higher than its corresponding PSA-Luc reporter IC₅₀. Overall, hits and analogs that inhibited and/or disrupted AR-TIF2 PPIs in the PPIB and M2H assays also blocked DHT-activated full length AR directed TA responses in C4-2 CRPC cells.

Inhibition of AR-V7 splice variant transcription activation: AR splice variants including AR-V7 are upregulated in CRPC patients that have relapsed on ADT (Bevan et al., Mol Cell Biol 1999, 19:8383-92; Callewaert et al., Cancer Res 2006, 66:543-53; Christiaens et al., J Biol Chem 2002, 277:49230-7; Wierman et al., Adv Physiol Educ. 2007, 31:26-33). To determine if AR-TIF2 PPI inhibitor/disruptor hits and analogs inhibited ligand-independent AR-V7 directed TA, the PSA6-6.1-Luc and UBE2C-Luc reporters were transfected into PC3-AR-V7-EGFP cells (Fancher et al., Assay Drug Dev Technol. 2019, 17:364-386). Ubiquitin-conjugating enzyme E2C (UBE2C) is a specific target gene of AR splice variants (Hu et al., Cancer Res. 2012, 72:3457-62; Xu et al., Cancer Res. 2015, 75:3663-71; Cao et al., Oncotarget 2014, 5:1646-56). The UBE2C luciferase reporter is driven by three AR-V7-specific promoter element repeats from the UBE2C gene (Xu et al., Cancer Res. 2015, 75:3663-71). The S1-1 hit and S1-2 analog inhibited the constitutive activation of both the PSA6-6.1-Luc and UBE2C-Luc reporters in PC3-AR-V7-EGFP cells with mid μM IC₅₀s (FIGS. 1, 9H, 9I). The S1-3, S1-4 and S1-5 analogs were not tested in the two AR-V7 reporter assays. The S2-6 hit produced IC₅₀s of 8 and 14.5 μM in the AR-V7 driven PSA6-6.1-Luc and UBE2C-Luc reporters respectively (FIG. 3). S2-6 analogs that were inactive in both AR-TIF2 PPIB assay formats were not tested in the two AR-V7 reporter assays. The S3-11 and S3-14 hits produced mid μM IC₅₀s of in the PSA-Luc AR-V7 reporter assay, the S3-12 and S3-15 analogs produced IC₅₀s in the low μM range, while the S3-13 and S3-17 analogs were less active with mid μM IC₅₀s, and the S3-21 analog was inactive at ≤100 μM (FIG. 6). The S3-11 and S3-14 hits produced mid μM IC₅₀s in the UBE2C-Luc AR-V7 reporter assay, as did the S3-12, S3-15, and S3-17 analogs (FIG. 6). The S3-13 and S3-21 analogs were inactive at ≤100 μM in the UBE2C-Luc AR-V7 reporter assay (FIG. 6). Cells were exposed to compounds at the indicated concentrations for 24h in the AR-V7 driven PSA-Luc and UBE2C-Luc reporter assays. Only the S1-2 analog exhibited activity in the cytotoxicity counter screen, producing an IC₅₀ of 45.9 μM, >3.5-fold higher than its corresponding IC₅₀s of 12.2 μM and 8.3 μM in the PSA-Luc and UBE2C-Luc reporter assays respectively. It is perhaps surprising that AR-TIF2 PPI inhibitor/disruptor hits and analogs inhibited constitutive TA driven by the AR-V7 splice variant that lacks a LBD, even though splice variants like AR-V7 also require CoAs like SRC-1 and TIF2 to activate transcription (Bevan et al., Mol Cell Biol 1999, 19:8383-92; Callewaert et al., Cancer Res 2006, 66:543-53; Christiaens et al., J Biol Chem 2002, 277:49230-7; Lavery et al., Biochem J 2005, 291:449-64; Ueda et al., J Biol Chem 2002, 277:38087-94). One potential mechanism is that the molecules may disrupt AR-V7s interactions with full length AR (Xu et al., Cancer Res 2015, 75:3663-71; Lv et al., J Clin Invest 2021, 131). Nevertheless, novel small molecules that inhibit CoA recruitment and AR-TA by either or both the AF-2 and/or AF-1 surfaces of full length AR and/or AR splice variants would be desirable leads for development into CRPC therapies.

Growth inhibition assays in AR positive and negative prostate cancer cell lines: To evaluate the cytotoxicity of AR-TIF2 PPI hits and analogs towards prostate cancer cells after longer compound exposure times, AR-TIF2 PPI inhibitor/disruptor hits and analogs were tested at the indicated concentrations (≤100 μM) for 72 h in established growth inhibition assays conducted in TIF2 expressing PC cell lines that are positive (LNCaP, C4-2, & 22Rv1 cells) or negative (PC-3 & DU-145) for AR (FIGS. 1, 3, 6, 9J, 9K, 9L) (Fancher et al., Assay Drug Dev Technol. 2016, 14:453-477; Fancher et al., Assay Drug Dev Technol. 2019, 17:364-386). The S1-1 hit produced calculable GI₅₀s in the 29 to 56 μM range against AR positive PC cell lines, and GI₅₀s of 70 μM and >100 μM respectively in AR negative PC-3 and DU-145 cell lines (FIGS. 1, 9J). The S1-3 analog also exhibited differential cytotoxicity in AR positive PC cell lines, while S1-2 and S1-4 analogs were equipotent against all 5 PC cell lines. The S1-5 analog did not achieve ≥50% growth inhibition in any PC cell line at ≤100 μM (FIG. 1). The S2-6 hit exhibited differential cytotoxicity in AR positive PC cell lines with GI₅₀s in the 14-19 μM range but failed to achieve ≥50% growth inhibition in AR negative cell lines at ≤100 μM (FIGS. 3, 9K). Similarly, the S2-7 analog produced GI₅₀s in the 30-64 μM range in AR positive PC cell lines, and GI₅₀s˜94 μM in AR negative cell lines (FIG. 3). The S2-8, S2-9, and S2-10 analogs failed to achieve ≥50% growth inhibition in any PC cell line at ≤100 μM (FIG. 3). The S3-11 and S3-14 hits also exhibited differential cytotoxicity in AR positive PC cell lines relative to AR negative cell lines (FIGS. 6, 9L). However, the S3-12, S3-13, S3-15, and S3-17 analogs were roughly equipotent against all 5 PC cell lines (FIG. 6). The S3-21 analog exhibited differential cytotoxicity in AR positive PC cell lines relative to AR negative cell lines (FIG. 6). Overall, 72 h exposure to compounds from the three series that are active in the AR-TIF2 PPIB, M2H, and AR or AR-V7 reporter assays also inhibited the growth of PC cell lines, with many exhibiting differential cytotoxicity towards AR positive cell lines (FIGS. 1, 3, 6, 9J, 9K, 9L).

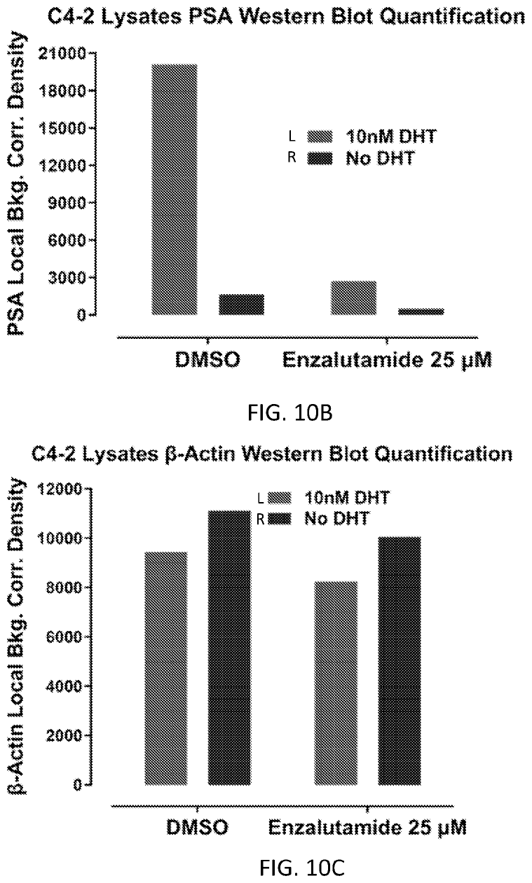

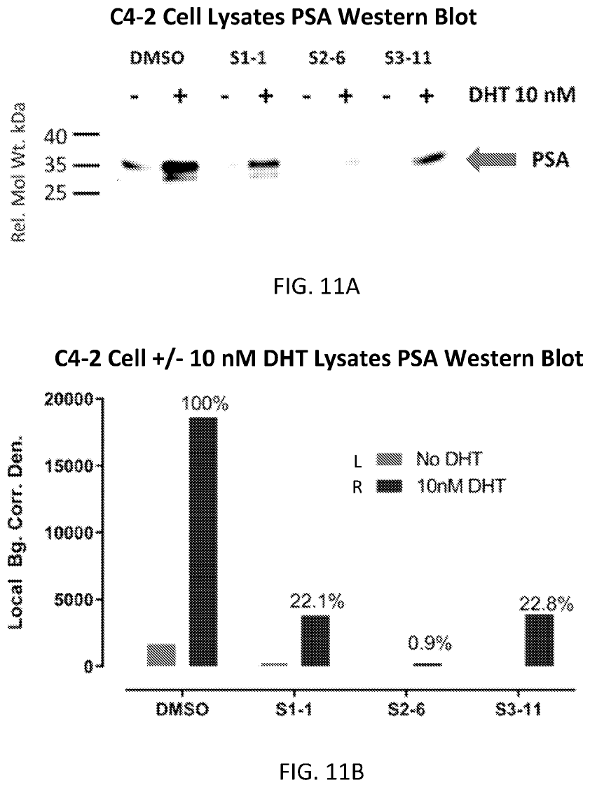

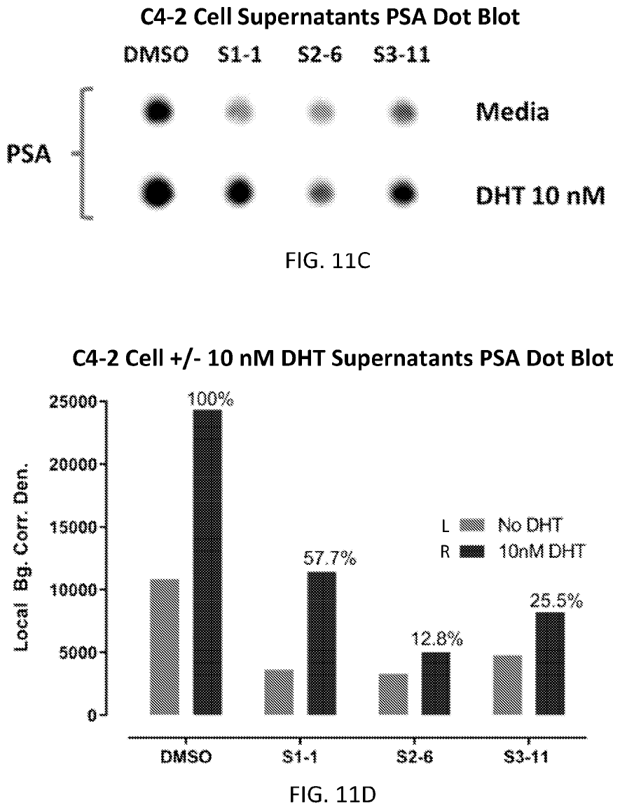

Inhibition of AR regulated prostate specific antigen biomarker expression and secretion: Screening and early detection of PC involves measurement of elevated serum levels of the PSA biomarker. PSA is a member of the kallikrein family of serine proteases (kallikrein 3) produced by prostatic luminal epithelial cells and widespread PSA testing is credited with the 45-70% decrease in PC mortality observed in the 1990s (Salami et al., Ther. Adv. Urol. 2022, 14:1-18). However, PSA is organ-specific but not cancer-specific, and serum PSA can be elevated in benign conditions like benign prostatic hyperplasia (BPH) and prostatitis leading to unnecessary biopsies, over diagnosis, and over treatment of indolent diseases (Ibid.). Despite these limitations, PSA remains the most widely used oncologic biomarker which has revolutionized PC screening and early detection, reducing the proportion of PC patients presenting with advanced disease (Ibid.). A combination of SDS-PAGE, western blots probed with specific antibodies to PSA and β-actin (FIG. 10A) and scanning densitometry were used to compare the levels of the cell associated PC biomarker PSA (FIG. 10B) and the R-actin housekeeping protein (FIG. 10C) in C4-2 cells cultured for 24 h in the presence or absence of 10 nM DHT after pre-exposure to DMSO or 25 μM enzalutamide for 3 h. The bicinchoninic acid (BCA) assay was used to determine the protein concentrations of C4-2 cell lysates and adjusted them to equal protein concentrations for loading onto SDS-PAGE gels that were transferred to western blots for probing with specific antibodies to PSA and β-actin (FIGS. 10A-10C). Compared to untreated controls, exposure of C4-2 cells to 10 nM DHT for 24 h substantially increased PSA levels by 12.3-fold over endogenous media controls (FIGS. 10A, 10B). In marked contrast, exposure to 10 nM DHT for 24 h did not substantially alter expression levels of β-actin compared to media controls (FIGS. 10A, 10C). In C4-2 cells exposed to 25 μM of the ADT drug enzalutamide for 3 h prior to the addition of media or 10 nM DHT for an additional 24 h, enzalutamide substantially reduced both the endogenous and DHT-enhanced PSA expression levels by 3.3-fold and 7.5-fold respectively (FIGS. 10A, 10B). Exposure to 25 μM enzalutamide did not substantially alter either the endogenous or DHT-treated expression levels of R-actin (FIGS. 10A, 10C). Across all treatment conditions the relative expression of the R-actin housekeeping protein was on average 0.98±0.17 indicating that the application of the BCA protein assay to determine and equalize protein loading was accurate and effective (FIGS. 10A, 10C). Exposure of C4-2 cells to the ADT drug enzalutamide effectively reduced both the endogenous and DHT-enhanced expression of the PC biomarker PSA. Compounds from the three chemical series that inhibited DHT-induced PSA-Luc reporter activity (FIGS. 1, 3, 6, 9G) were evaluated to determine whether they would also reduce PSA expression and/or secretion by C4-2 CRPC cells (FIGS. 11A-11D). Conditioned media collected from the same C4-2 cultures was centrifuged then transferred to dot blots that were probed with the same PSA antibody and scanning densitometry was used to quantify the relative levels of secreted PSA (FIG. 11C, 11D). Compared to untreated controls, exposure of C4-2 cells to 10 nM DHT for 24 h substantially increased PSA levels in cells and conditioned media by 11.4-fold and 2.4-fold respectively. C4-2 cells were exposed to S1-1, S2-6, and S3-11 at 20 μM for 3 h prior to the addition of DMSO or 10 nM DHT and incubation for an additional 24 h. Consistent with their inhibition of the DHT-induced AR-driven PSA-Luc reporter activity (FIG. 9G), all 3 hits substantially reduced both endogenous and DHT-enhanced expression and secretion of the PSA PC biomarker by C4-2 CRPC cells (FIGS. 11A-11D).

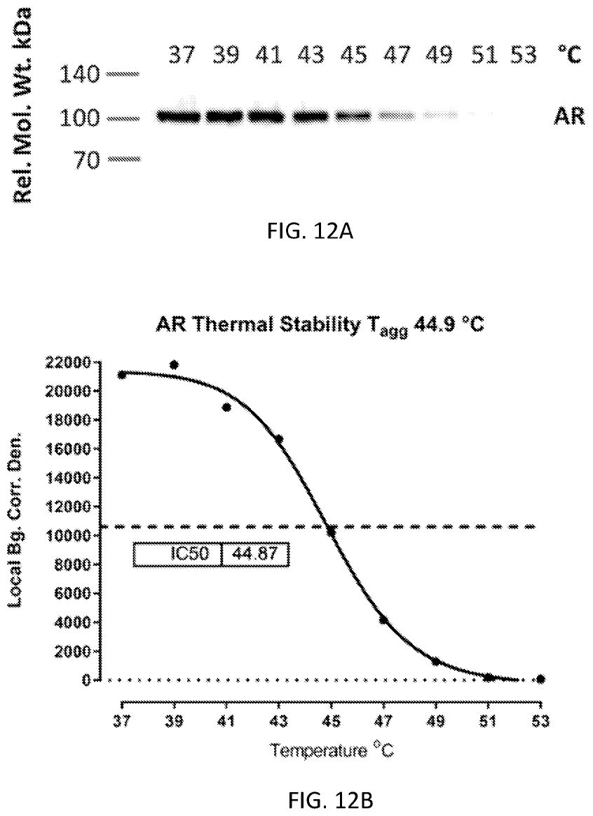

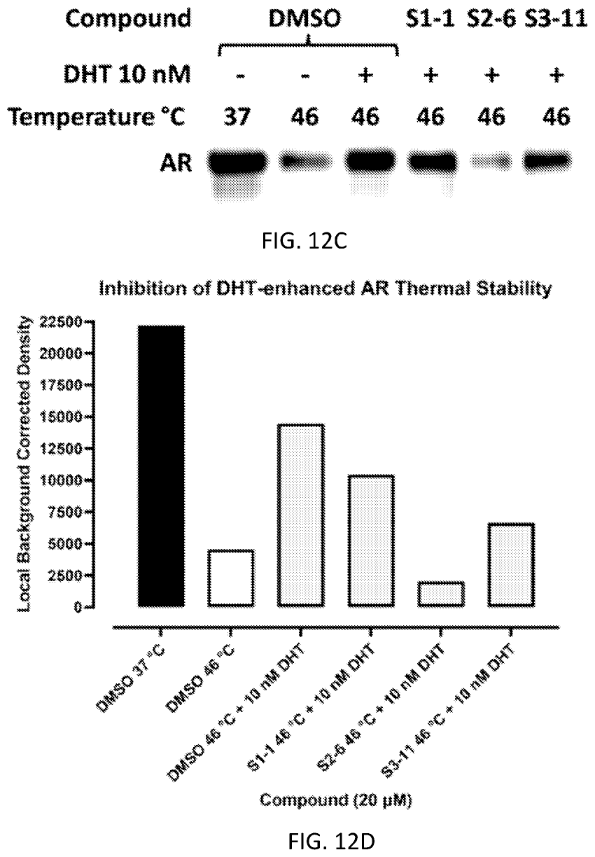

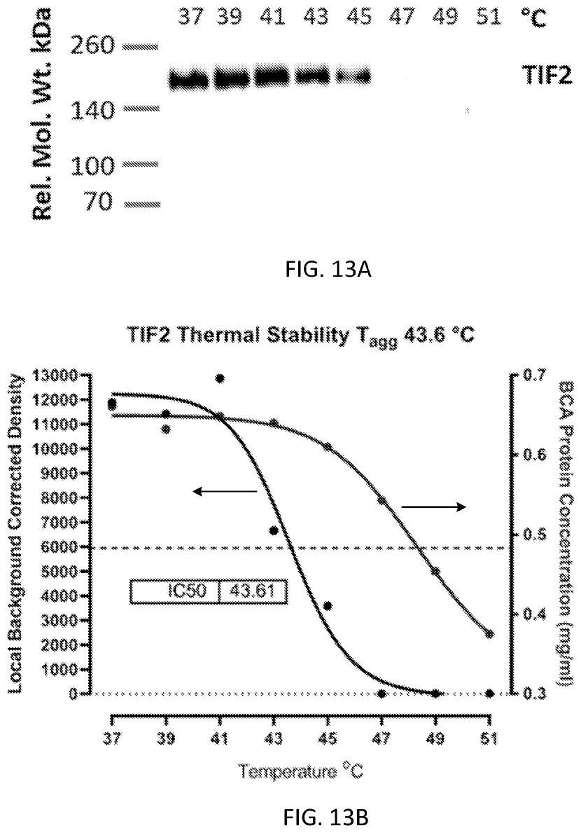

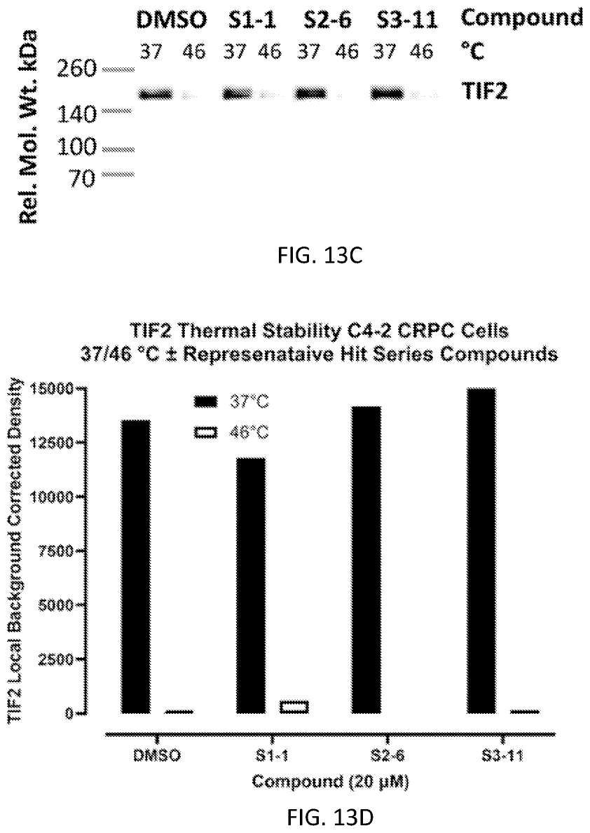

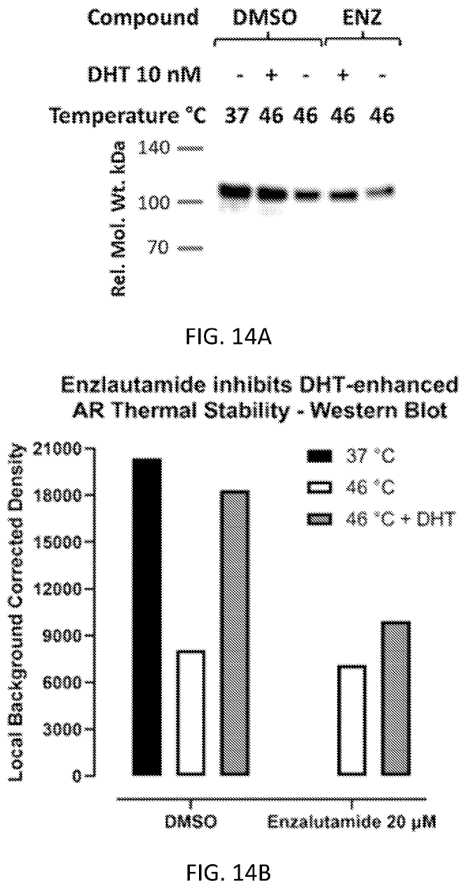

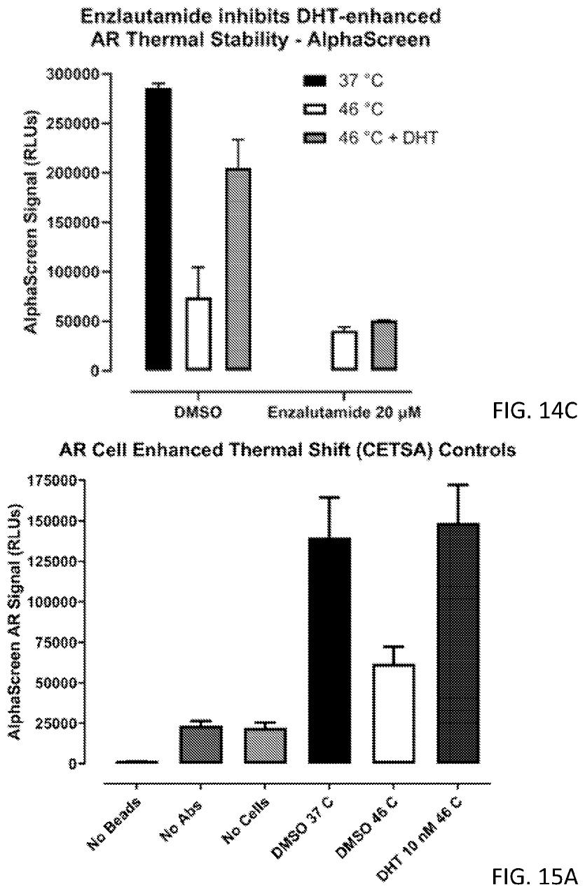

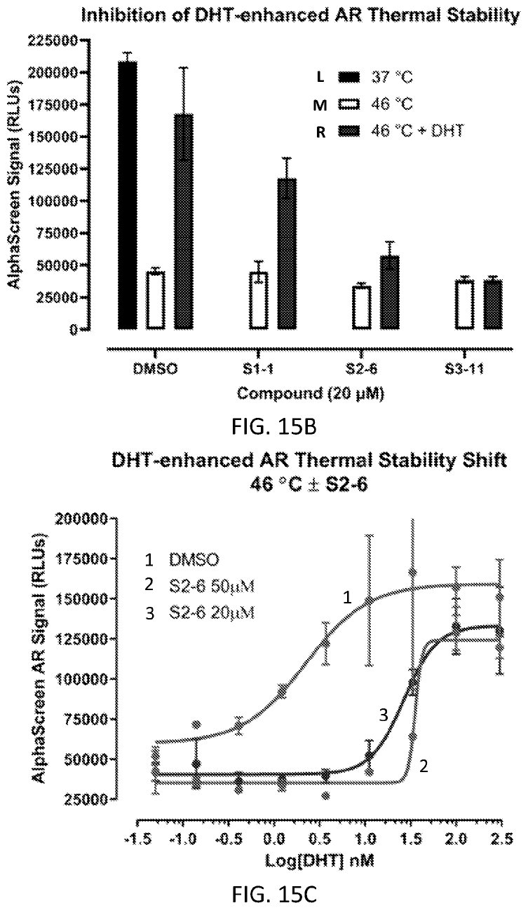

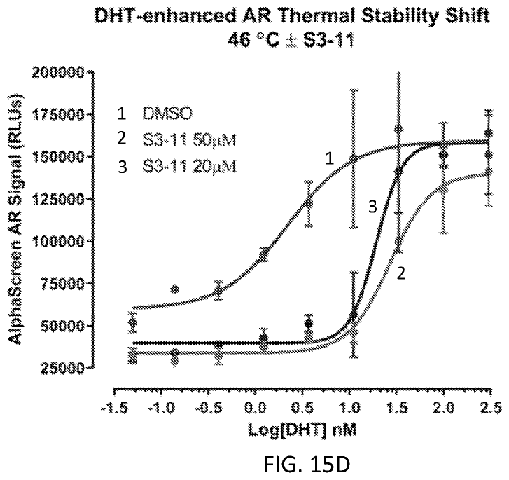

Cell enhanced thermal shift (CETSA) TIF2 and AR target engagement assays: Western blotting (FIGS. 12A-12D, 13A-13D, 14A, 14B) and AlphaScreen (FIGS. 14C, 15A-15D) cell enhanced thermal shift target engagement assays (CETSA) in C4-2 CRPC cell lines were used to determine if hits bound to either AR or TIF2 (Shaw et al., Sci Rep 2018, 8(1):163-174); Henderson et al., SLAS Discovery 2020, 25(2):137-147). C4-2 CRPC cells were subjected to heat shock in a PCR instrument where a temperature gradient was ramped up at 2° C. intervals from 37° C. to 53° C. to denature and aggregate proteins. The amount of soluble AR or TIF2 detected in cell lysates after centrifugation was determined by SDS-PAGE and western blots probed with specific antibodies to TIF2 (FIGS. 13A-13D) or AR (FIGS. 12A-12D, 14A, 14B) and quantified by densitometry. On western blots of lysates prepared from C4-2 cells that were heat shocked and probed with a specific TIF2 antibody (FIG. 13A), the amount of soluble TIF2 was reduced at increasing temperatures and characterized by a 50% reduction T_{agg} value of 43.6° C. (FIG. 13B). A a 5 min heat shock denaturation temperature of 46° C. was used to determine if pre-exposure of C4-2 cells to hit compounds would enhance TIF2 thermal stability (FIGS. 13C, 13D). Pre-exposure of C4-2 cells to 20 μM of the S1-1, S2-6, or S3-11 hits for 1 h at 37° C. prior to heat shock at 46° C. did not enhance TIF2 thermal stability over DMSO (FIGS. 13C, 13D), suggesting that they do not bind to or engage TIF2.

AR exhibited a characteristic reduction in soluble protein at increasing temperatures with a 50% reduction T_{agg} value of 44.9° C. (FIGS. 12A, 12B). Similar to a published method (Shaw et al., Sci Rep. 2018, 8:163-174), a 5 min heat shock denaturation temperature of 46° C. was used to determine the effects of compound exposure on the thermal stability of AR in C4-2 cells (FIGS. 12C, 12D, 14A, 14B). Pre-exposure of C4-2 cells to 10 nM of the AR agonist DHT for 1 h at 37° C. prior to heat shock at 46° C. substantially enhanced the amount of soluble AR in cell lysates compared to untreated and/or DMSO treated cells (FIGS. 12C, 12D, 14A, 14B). As reported previously (Ibid.), exposure of C4-2 cells to the AR antagonist enzalutamide prior to heat shock did not increase the thermal stability of AR at 46° C., but blocked DHT-enhanced AR thermal stability thereby confirming enzalutamide AR target engagement (FIGS. 14A, 14B). Pre-exposure of C4-2 cells to 20 μM of S1-1, S2-6, or the S3-11 hit for 1 h at 37° C. prior to heat shock at 46° C. did not stabilize AR, but blocked DHT-enhanced AR stabilization (FIGS. 12C, 12D). At 20 PM, the S2-6 hit blocked DHT-enhanced AR thermal stabilization below DMSO baseline levels at 46° C., while the S3-11 and S1-1 hits only partially blocked DHT-enhanced AR stabilization to levels below DHT but above DMSO controls (FIGS. 12C, 12D). To provide a CETSA assay with higher throughput and capacity than western blotting a modified AlphaScreen® AR CETSA assay was used where one of the antibodies in the published pair was changed (Ibid.) (FIG. 14C, 15A-15D). Consistent with the existing AlphaScreen® AR CETSA (Ibid.) and the AR western blotting data (FIGS. 12A-12D, 13A-13D), pre-treatment of C4-2 cells with 10 nM DHT for 1 h at 37° C. prior to heat shock at 46° C. enhanced the thermal stability of AR in cell lysates compared to DMSO treated cells (FIG. 14C, 15A-15D). Similarly, pre-treatment of C4-2 cells with enzalutamide prior to heat shock did not enhance AR thermal stability in the AlphaScreen® assay, but did block DHT-enhanced AR stabilization (FIG. 14C) (Ibid.). In agreement with the AR western blotting data (FIGS. 12A-12D, 14A, 14B), exposure of C4-2 cells to 20 μM of S1-1, S2-6, or the S3-11 hits for 1 h at 37° C. prior to heat shock did not stabilize AR in the AlphaScreen® assay but blocked DHT-enhanced AR thermal stabilization (FIG. 15B). To determine the isothermal concentration fingerprint of DHT (Ibid.), C4-2 cells were pre-exposed to different agonist concentrations prior to heat shock at 46° C. (FIGS. 15C, 15D). DHT exhibited an EC₅₀ of 2.22 nM for in-cell AR thermal stabilization (FIGS. 15C, 15D). Pre-treatment of C4-2 cells with 20 or 50 μM of S2-6 reduced the maximum efficacy of DHT-enhanced AR thermal stabilization and right shifted the DHT EC₅₀ by >10-fold to 26.6 nM and 35.5 nM respectively (FIG. 15C). Pre-treatment of C4-2 cells with 20 or 50 μM of S3-11 also right shifted the DHT EC₅₀ for in-cell AR thermal stabilization by ≥10-fold to 19.3 nM and 27.0 nM respectively, and at 50 μM reduced the maximum efficacy of DHT (FIG. 15C). The ability of S2-6 and S3-11 to decrease the efficacy and right shift the DHT EC₅₀ for enhancing AR thermal stability in heat shocked C4-2 cells are consistent with the effects of a negative allosteric modulator (Conn et al., Nat Rev Drug Discov. 2009, 8:41-54). Although S1-1 also blocked the ability of DHT to enhance AR thermal stability in heat shocked C4-2 cells it was less effective than S2-6 or S3-11 (FIGS. 12C, 12D, 15B). Based on these data (FIGS. 12A-12D, 13A-13D, 14A-14C, 15A-15D), the hit series compounds bind to AR where they behave as negative allosteric modulators (AMs) that inhibit coactivator recruitment and transcriptional activation.

### Example 3

### Synthesis and Evaluation of Compounds

The core structure (2,3,4,5-tetrahydrobenzo[f][1,4]oxazepin-9-ol) of hdyrobenzo-oxazepines is shown in Scheme 1.

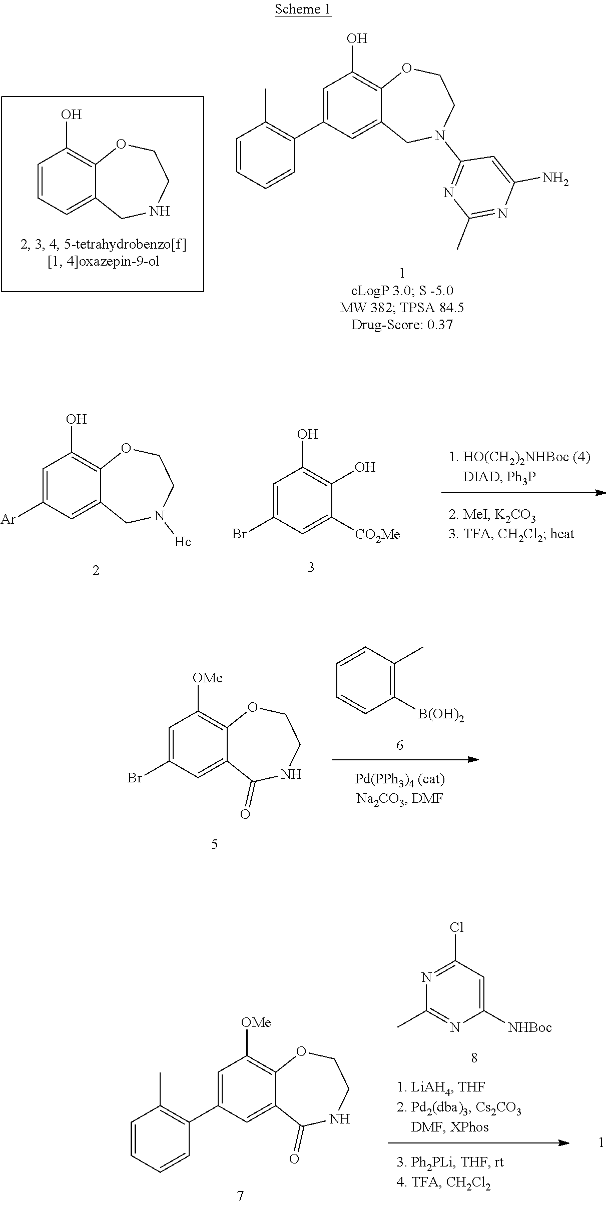

These compounds can be synthesized by subjecting commercially available methyl salicylate 3 to a Mitsunobu reaction with alcohol 4, which engages the more acidic ortho-hydroxy group, followed by methylation of the meta-hydroxy and Boc-cleavage to generate amide 5 after thermal amide ring closure. A Suzuki coupling with boronic acid 6 provides intermediate 7 which is reduced with LiAlH₄ to give the benzylic amine intermediate. Preparation of compound 1 is then completed by a Buchwald-Hartwig coupling with pyrimidine 8 followed by demethylation (Wipf, et al., J Org Chem 2000, 65(20):6319-6337) and Boc cleavage. Substituents on the core scaffold can be readily modified by exchanging boronic acid 6 and chloride 8 with alternative aryl and heteroaryl reagents, allowing for an extension of the SAR without the need to develop a new synthetic route.

The core structure (an N-(1,2,4-thiadiazol-5-yl)piperidine-1-carboxamide) of the thiadiazol-5-piperidine carboxamides is shown in Scheme 2.

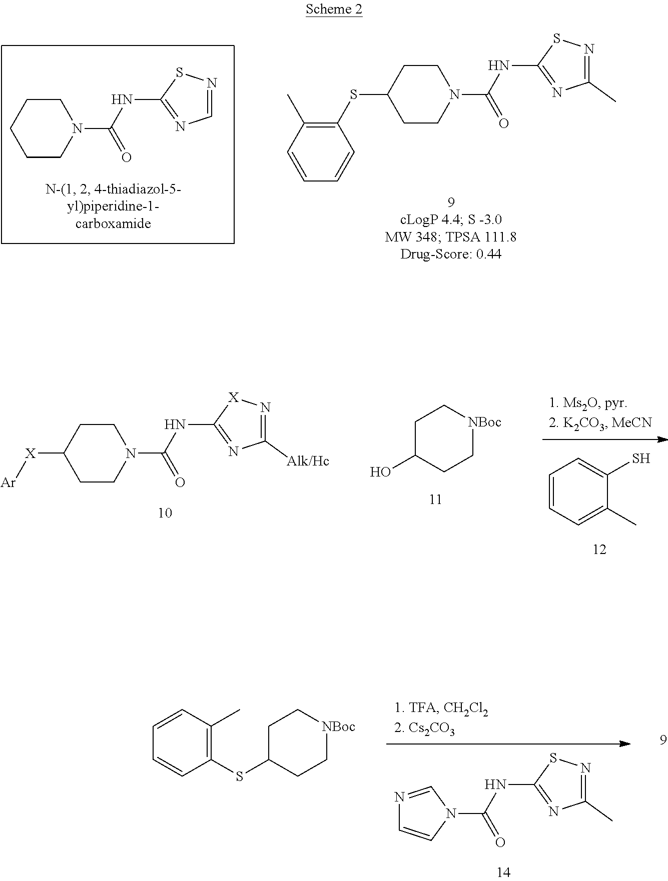

Compound 9 can be synthesized by mesylating the commercially available piperidine 11 and displacing the mesylate with commercially available thiol 12 to generate thioether 13. Boc-deprotection with TFA and condensation with activated urea 14 completes the synthesis of 9.

The series 3 compounds feature two closely related core structures (2-phenylindole and 3-phenylindole) shown in Scheme 3.

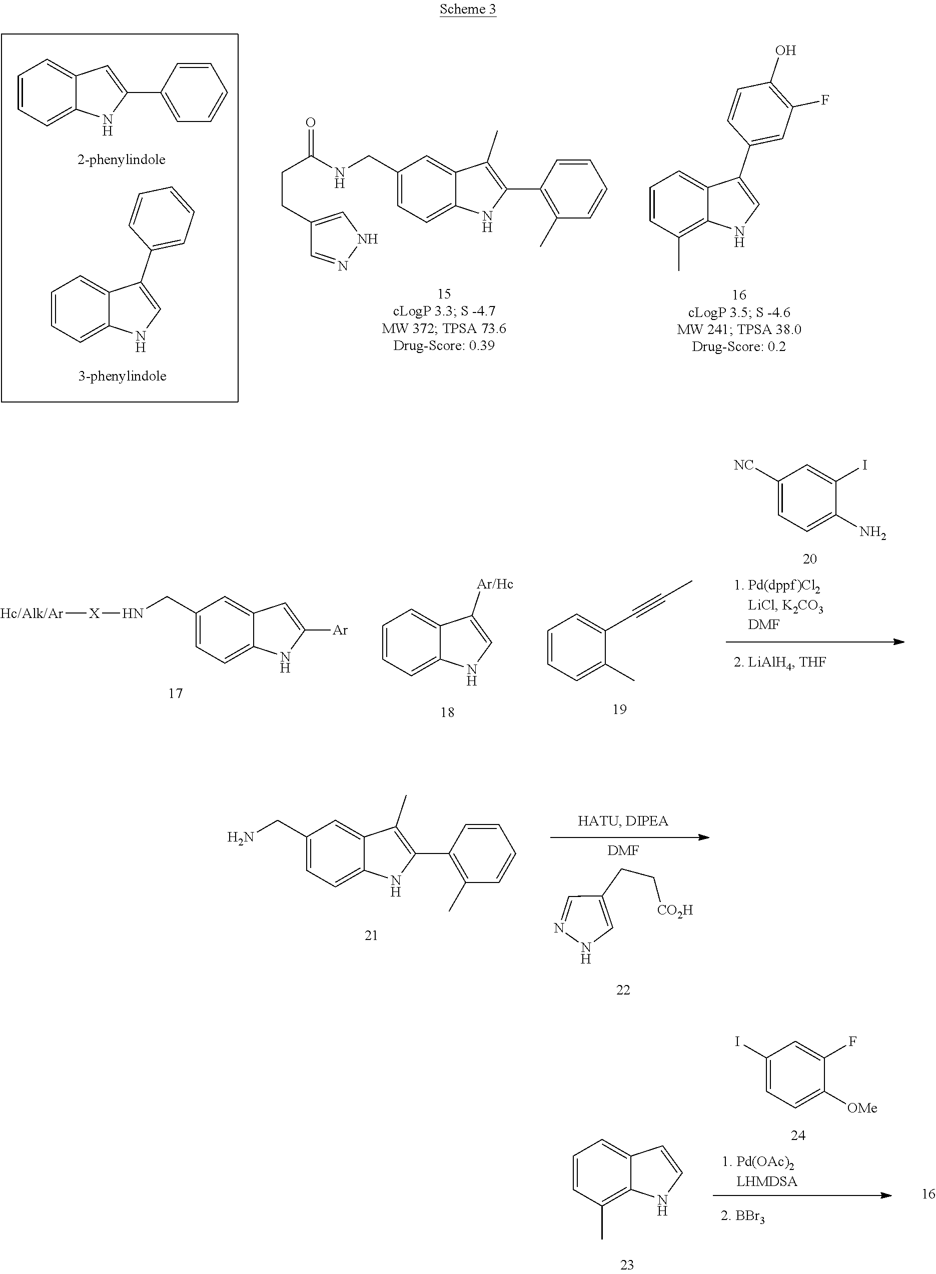

Compound 15 is synthesized using a Larock indole synthesis from alkyne 19 and ortho-iodoaniline 20 to access intermediate 21 after reduction of the nitrile. Simple amide coupling with acid 22 completes the synthesis of 15. There are many methods available to generate hit 16, and a practical route involves the coupling (Mohr et al., ACS Catalysis 2020, 10(4):2713-2719) of indole 23 with aryliodide 24 followed by demethylation. Alternative methods use the Fischer indole synthesis or the inventors' own synthesis (Xu et al., Org Biomol Chem 2017, 15(34):7093-7096). Both synthetic strategies readily lend themselves to the generation of analogs of 15 and 16.

Synthesized analogs are profiled in the biochemical and cell-based assays disclosed in Example 1.

Drug combination matrices (DCMs) can be prepared for prostate cancer active compounds and selected analogs (Close et al., SLAS Discov 2018, 24:242-263; Fent et al., J Chem Biol 2015, 8:79-93; Kochanek et al., SLAS Discov 2019, 24(6):653-668; Close et al., SLAS Discov 2021, 26(5):712-729). It is anticipated that pairwise DCMs for ≤30 drugs will cover the PC DC space −20 FDA approved drugs and 10 analogs. DCs will be arrayed in 4×4 DCMs, 20 DCMs per plate. Labeled analogs may be prepared: e.g., radiolabeled with tritium or other radioisotopes, fluorescent (BODIPY, etc.) or photoaffinity (diazo or diaziridines tags) to aid in target ID, metabolic profiling, pharmacokinetic (PK) studies, etc. Standard pharmaceutical profiling may be performed, such as solubility, log P and microsomal stability, and studies used to interpret cellular activity or lack thereof, prioritize compounds and aid in the design of more efficacious analogs into leads.

The in vitro stability (microsomal/hepatocyte metabolism) and absorption potential (Caco2 permeability) of lead compounds will be performed as described, and the half-life and intrinsic clearance parameters will be calculated. Bioavailability, drug distribution (plasma protein binding), and excretion will be tested in in vivo mouse studies. To assess bioavailability, both intravenous (IV) and oral gavage (PO) dosing of the lead compounds (5 mg/kg) will be performed. Compounds will be dissolved in normal saline or in a mixture of Cremophor® emulsifier (BASF Corporation, Florham Park, NJ) and alcohol and diluted in normal saline and administered to mice IV or PO. A set of mice (n=3) will be euthanized by CO₂ inhalation and tissues will be collected (blood, liver, kidney, brain, lungs), at various time points (15, 30, 60, 240, 360, 480, 720, 1440 minutes). Blood samples will be taken by cardiac puncture, transferred to microcentrifuge tubes, and plasma separated by centrifugation (12,000 g for 5 min) at room temperature and frozen at −80° C. Tissue samples will be frozen and stored at −80° C. Urine and fecal samples will be collected from mice in the 1440-minute group that will be housed in metabolic cages. Plasma, urine, and fecal samples will be assayed for intact parent compound using our validated UPLC-MS-MS methods. Bioavailability will be predicted based on IV clearance parameter (Bioavailability=(1−(CL/Q), where CL is the total body clearance of the compound (Dose/area under the plasma concentration vs time (AUC)) and Q is the hepatic blood flow. Bioavailability will also be calculated as (AUC) for PO/AUC for IV dosing. The tissue concentrations will be measured by pulverizing the tissues and extracting the chemical using methanol, as described previously (Feturi et al., Pharm Res 2020, 37(11)). To evaluate excretion, urine samples collected after IV administration will be analyzed for the parent compound. Renal clearance will be calculated as the amount excreted in the urine at 24 hrs/AUC. A comparison of the renal clearance to the total body clearance will provide an estimate of relative contribution of kidney and liver to the clearance of the lead compounds. Safety will be assessed at 4 doses after a single dose study. Doses will be based on the PK profiles of the lead compounds and their potency/efficacy in bioassays. Hematological, renal, and liver injury parameters will be assessed for the leads. Hematological complete blood counts (CBC) will be performed. For kidney, 24-hour urine collections will be performed to measure urinary creatinine levels (Cr urine), and blood samples will be collected for serum creatinine levels (Cr serum). Creatinine Clearance (ml/min) will be calculated (Cr urine×Urine volume)/Cr serum)/1440). Liver enzymes AST, ALT, and bilirubin levels will be measured as liver injury markers.

The efficacy of molecules with favorable PK and safety profiles will be evaluated in 3 different subcutaneous CRPC tumor models. C4-2 tumors in castrated mice are a widely used CRPC tumor model (Thalmann et al., Cancer Res 1994, 54(10):2577-2581). VCaP tumors in mice provids a model for AR-V7-positive CRPC (Korenchuk et al., In Vivo 2001, 15(2):163-168; Udayakumar et al., Mol Cancer Ther 2016, 15(6):1353-1363). 22Rv1 tumors provide a CRPC model resistant to enzalutamide (Martin et al., Mol Oncol 2014, 9(3):628-639). For each model, tumor cells will be implanted subcutaneously in castrated mice. After the tumor volume reaches 300 μL, animals will be randomized into 2 experimental groups, which will be treated with daily IP injection of vehicle or an analog at 50 mg/kg or a dose based on the PK studies. Tumor volumes will be measured twice a week, until 100 days post-castration or when any one tumor length reaches 20 mm or a volume of 2 cm³.

In view of the many possible aspects to which the principles of the disclosed invention may be applied, it should be recognized that the illustrated aspects are only preferred examples of the invention and should not be taken as limiting the scope of the invention. Rather, the scope of the invention is defined by the following claims. We therefore claim as our invention all that comes within the scope and spirit of these claims.

1. A method of modulating androgen receptor-mediated activity, comprising:
contacting androgen receptor with an effective amount of a compound, wherein the compound is a hydrobenzo-oxazepine, a thiadiazol-5-piperidine carboxamide, a fluorophenyl-methyl-indole, a phenyl-methyl-indole, a heteroaliphatic- or heteroaryl-substituted methyl-indole, or any combination thereof, thereby inhibiting androgen receptor-coactivator (AR-CoA) protein-protein interaction (PPI) complexes, inhibiting prostate specific antigen (PSA) expression in prostate epithelial cells and/or prostate cancer cells, inhibiting PSA secretion by prostate epithelial cells and/or prostate cancer cells, inhibiting AR-mediated PSA promoter-driven transcription in prostate cancer cells, inhibiting androgen receptor splice variant 7 (AR-V7)-mediated PSA promoter driven transcription in prostate cancer cells, inhibiting ubiquitin conjugating enzyme E2 C (UBE2C) promoter-driven transcription in prostate cancer cells, inhibiting growth of prostate cancer cells, or any combination thereof,
wherein the compound has a structure according to any one of formulas I-III: 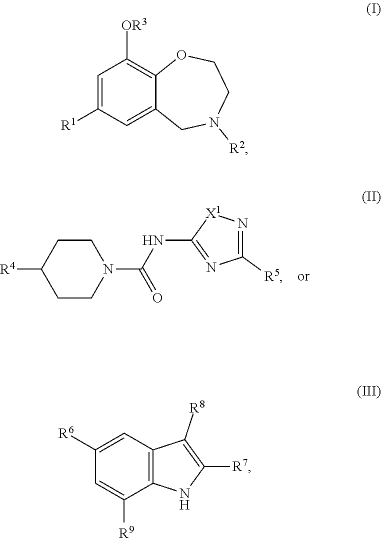
wherein
R¹ is aryl or heteroaryl;
R² is a heterocycle;
R³ is H;
X¹ is S, O, or N;
R⁴ is —X²—R^{a} or halo, where X² is CH₂, S, or O, and R^{a} is aryl or heteroaryl;
R⁵ is aliphatic or a heterocycle;
R⁶ is —CH₂N(H)Y(CH₂)ₘ—R^{b}, H, halo, or alkyl, where Y is C(O) or S(O)₂, R^{b} is a heterocycle or —N(H)Y(CH₂)ₘCH₃, and each m independently is 0, 1, 2, or 3;
R⁷ is aryl, heteroaryl, H, or alkyl:
R⁸ is alkyl, aryl, a heterocycle, or H; and
R⁹ is H, alkyl, or —CH₂N(H)Y(CH₂)ₘ—R,
wherein at least one of R⁶ and R⁹ is not H,
at least one of R⁷ and R¹ is not H, and
one of R⁶-R⁹ is —CH₃.

2. (canceled)

3. The method of claim 21, wherein:
(i) R¹ is phenyl or thiophenyl and R² is thiazolyl or pyrimidinyl; or
(ii) R^{a} is phenyl, imidazolyl, or pyrrolidinyl; or
(iii) R^{b} is diazolyl, pyrimidinyl, pyridinyl, 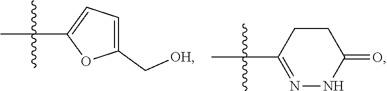 [SMILES: *c1ccc(CO)o1 ; *C1=NNC(=O)CC1]
or —N(H)C(O)CH₃; or
(iv) R⁷ is 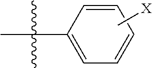
where X is halo; or
(v) R⁸ is —CH₃, 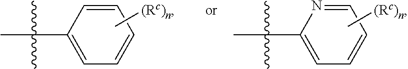
where n is 1 or 2, and each R^{c} independently is halo, —OR^{d}, —C(O)NHR^{d}, or aminoalkyl, where each R^{d} independently is H or C₁-C₅ alkyl; or
(vi) any combination of two or more of (i)-(v).

4. The method of claim 1, wherein the compound has a structure according to formula III where:
when R⁶ is H and R⁹ is methyl, then R⁷ is H and R⁸ is aryl or heteroaryl; or
when R⁶ is aryl and R⁹ is H, then R⁷ is aryl and R⁸ is methyl; or
when R⁶ is alkyl and R⁹ is H, then R⁷ is H and R⁸ is methyl; or
when R⁶ is halo and R⁹ is methyl, then R⁷ and R⁸ are not H.

5. The method of claim 1, wherein:
(i) the compound has a structure according to formula I 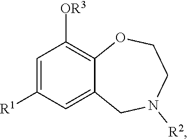
wherein
R¹ is 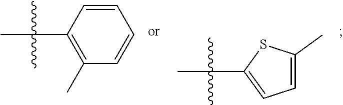
R² is 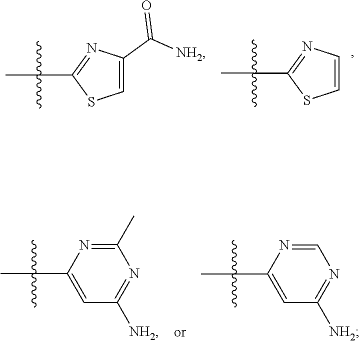
and R³ is H; or
(ii) the compound has a structure according to formula II 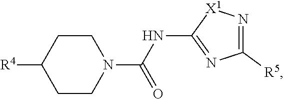
wherein
X¹ is S, R⁵ is —CH₃, R⁴ is —S—R^{a}, —CH₂—R^{a}, or F, and R^{a} is 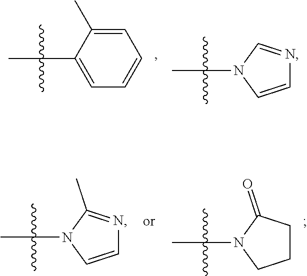
or
(iii) the compound has a structure according to formula III 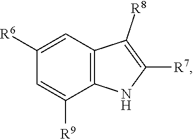
wherein
R⁶ is —CH₂N(H)C(O)(CH₂)ₘ—R^{b}, H, F, or —CH₃, where m is 0, 1, or 2, and R^{b} is 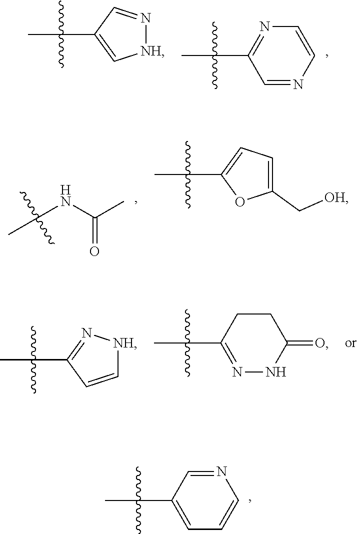
R⁷ is 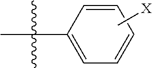
where X is halo, or R⁷ is H or —CH₂CH₃,
R⁸ is —CH₃ 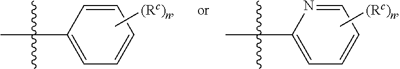
where n is 1 or 2, and each R^{c} independently is halo, —OR^{d}, —C(O)NHR^{d}, or aminoalkyl, where each R^{d} independently is H or C₁-C₃ alkyl, and
R⁹ is H, —CH₃, or —CH₂N(H)C(O)(CH₂)ₘ—R^{b}, where m is 3, and R^{b} is

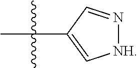 [SMILES: *c1cn[nH]c1]

6. The method of claim 1, wherein the compound has a structure according to formula IIIA, IIIB, or IIIC: 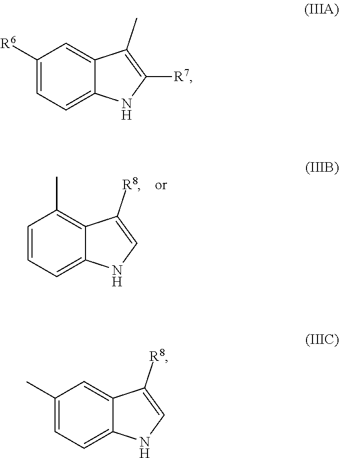
where
R⁶ is —CH₂N(H)Y(CH₂)ₘ—R^{b};
Y is CO or S(O)₂;
R^{b} is a heterocycle or —N(H)C(O)(CH₂)ₘCH₃;
R⁷ is aryl or heteroaryl; and
R⁸ is aryl or a heterocycle.

7. The method of claim 6, wherein:
(i) the compound has a structure according to formula IIIA, where R⁷ is fluorophenyl, H, or —CH₂CH₃, and R⁶ 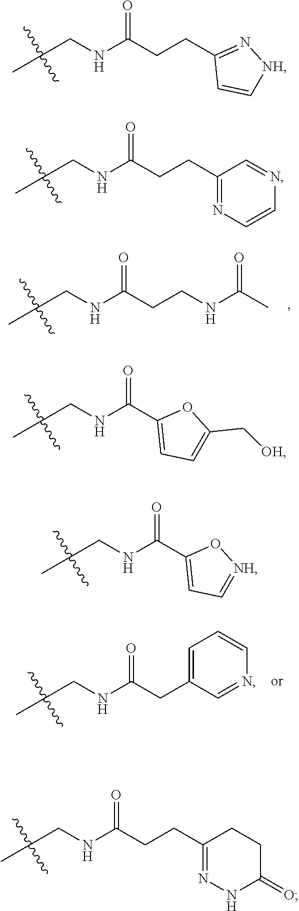
or
(ii) the compound has a structure according to formula IIIB or IIIC, where R⁸ is

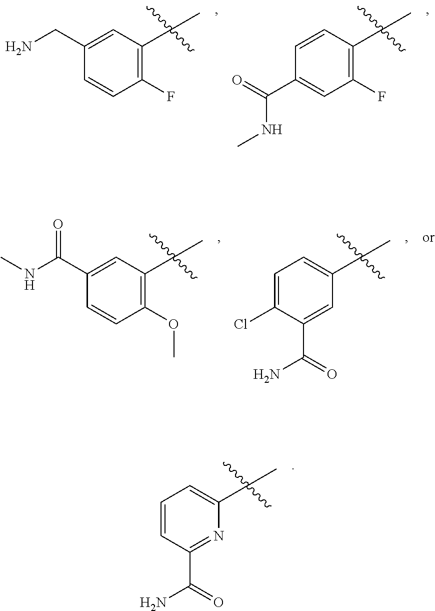

8. The method of claim 1, wherein the compound is: 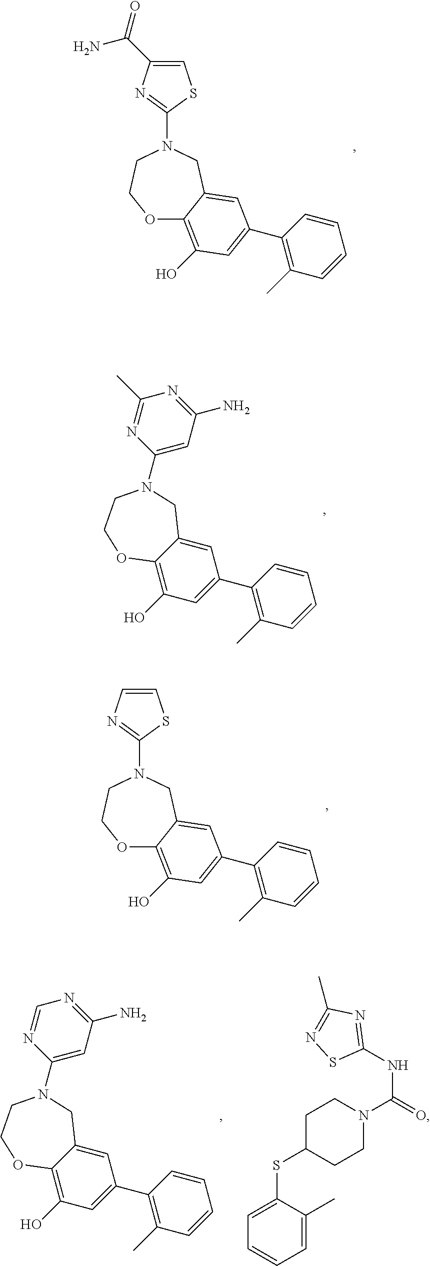 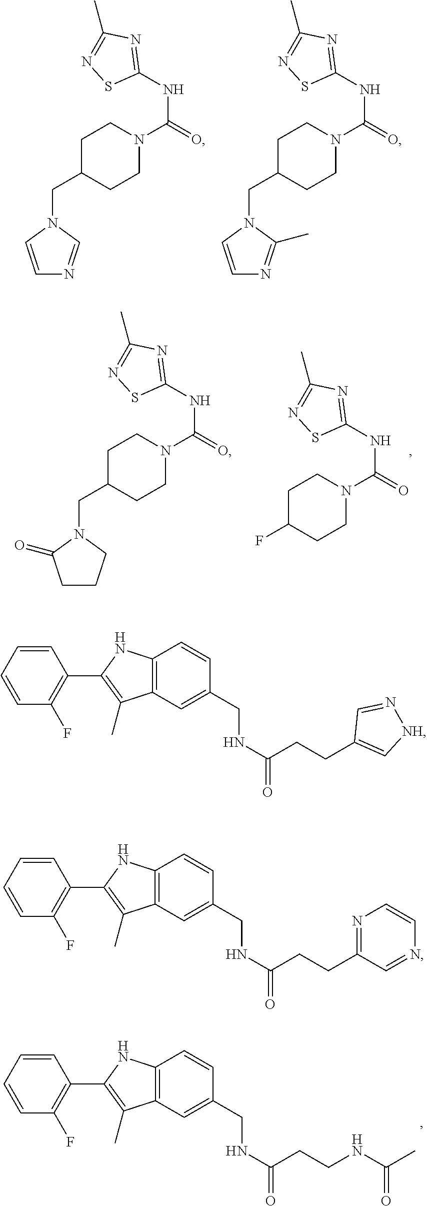 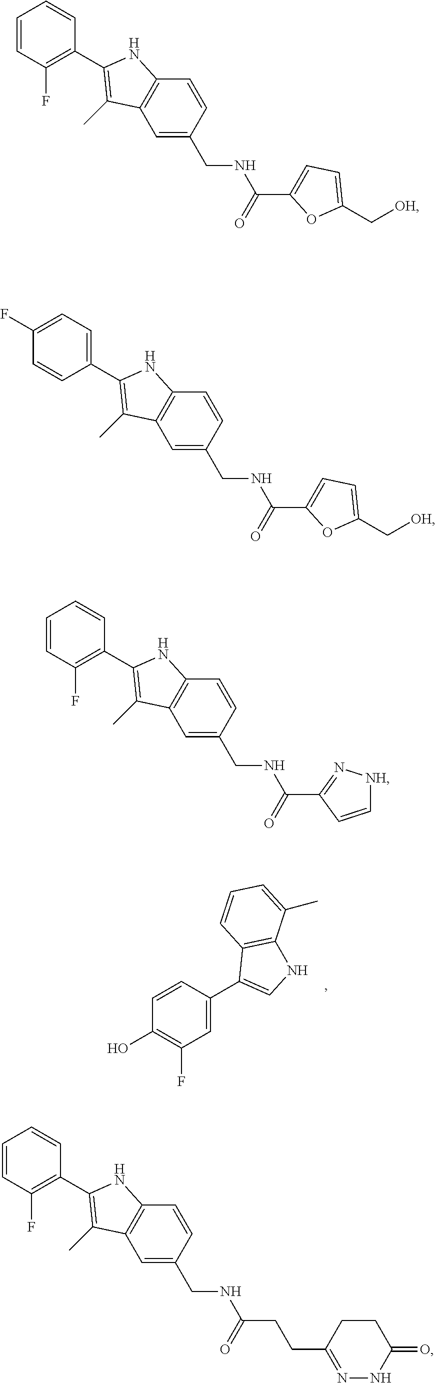 [SMILES: Cc1c(-c2ccccc2F)[nH]c2ccc(CNC(=O)c3ccc(CO)o3)cc12 ; Cc1c(-c2ccc(F)cc2)[nH]c2ccc(CNC(=O)c3ccc(CO)o3)cc12 ; Cc1c(-c2ccccc2F)[nH]c2ccc(CNC(=O)c3cc[nH]n3)cc12 ; Cc1cccc2c(-c3ccc(O)c(F)c3)c[nH]c12 ; Cc1c(-c2ccccc2F)[nH]c2ccc(CNC(=O)CCC3=NNC(=O)CC3)cc12] 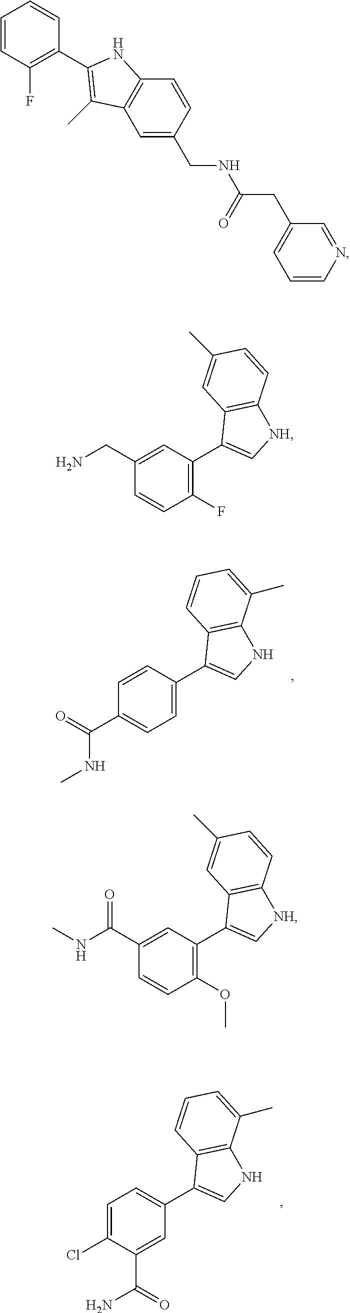 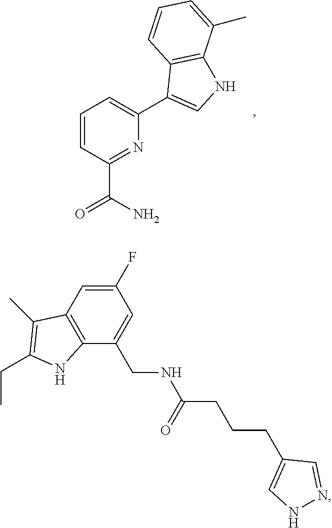
or any combination thereof.

9. The method of claim 1, wherein the compound is: 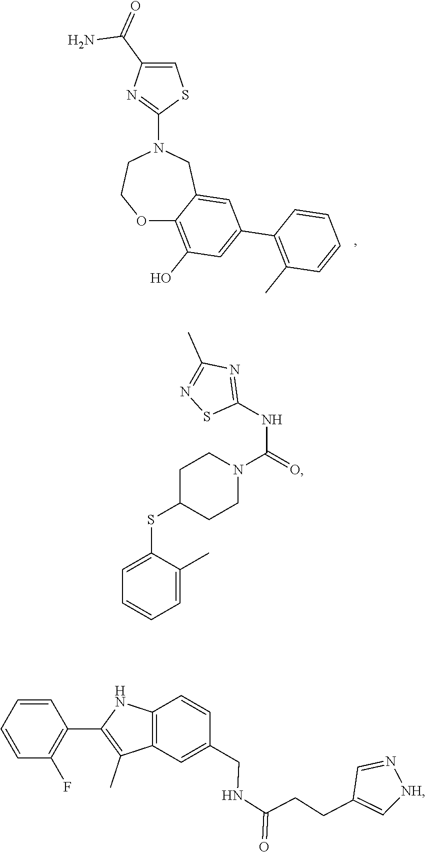 [SMILES: Cc1ccccc1-c1cc(O)c2c(c1)CN(c1nc(C(N)=O)cs1)CCO2 ; Cc1nsc(NC(=O)N2CCC(Sc3ccccc3C)CC2)n1 ; Cc1c(-c2ccccc2F)[nH]c2ccc(CNC(=O)CCc3cn[nH]c3)cc12]
or any combination thereof.

10. The method of claim 1, wherein inhibiting AR-CoA PPI complexes comprises:
(i) reducing formation of AR-CoA PPI complexes; or
(ii) disrupting formed AR-CoA PPI complexes; or
(iii) both (i) and (ii).

11. The method of claim 1, wherein the CoA comprises transcriptional intermediary factor 2 (TIF2), steroid receptor coactivator (SRC1), or a combination thereof.

12. The method of claim 1, wherein contacting is performed in vivo by administering the effective amount of the compound to a subject.

13. (canceled)

14. A method for treating prostate cancer in a subject, comprising administering to the subject a therapeutically effective amount of a compound, wherein the compound is a hydrobenzo-oxazepine, a thiadiazol-5-piperidine carboxamide, a fluorophenyl-methyl-indole, a phenyl-methyl-indole, a heteroaliphatic- or heteroaryl-substituted methyl-indole, or any combination thereof, the compound having a structure according to any one of formulas I-III: 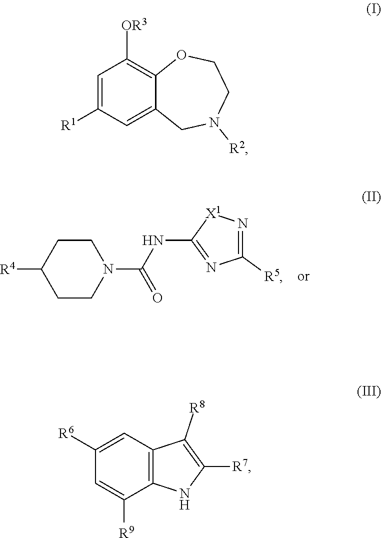
wherein
R¹ is aryl or heteroaryl;
R² is a heterocycle;
R³ is H;
X is S, O, or N;
R⁴ is —X²—R^{a} or halo, where X² is CH₂, S, or O, and R^{a} is aryl or heteroaryl;
R⁵ is aliphatic or a heterocycle;
R⁶ is —CH₂N(H)Y(CH₂)ₘ—R^{b}, H, halo, or alkyl, where Y is C(O) or S(O)₂, R^{b} is a heterocycle or —N(H)Y(CH₂)ₘCH₃, and each m independently is 0, 1, 2, or 3;
R⁷ is aryl, heteroaryl, H, or alkyl;
R⁸ is alkyl, aryl, a heterocycle, or H; and
R⁹ is H, alkyl, or —CH₂N(H)Y(CH₂)ₘ—R^{b},
wherein at least one of R⁶ and R⁹ is not H,
at least one of R⁷ and R¹ is not H, and
one of R⁶-R⁹ is —CH₃.

15. (canceled)

16. The method of claim 14, wherein:
(i) R¹ is phenyl or thiophenyl and R² is thiazolyl or pyrimidinyl; or
(ii) R^{a} is phenyl, imidazolyl, or pyrrolidinyl; or
(iii) R^{b} is diazolyl, pyrimidinyl, pyridinyl, 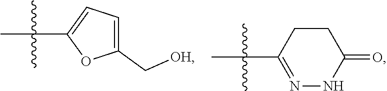 [SMILES: *c1ccc(CO)o1 ; *C1=NNC(=O)CC1]
or —N(H)C(O)CH₃; or
(iv) R⁷ is 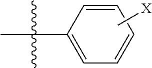
where X is halo; or
(v) R¹ is —CH₃, 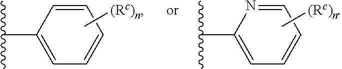
where n is 1 or 2, and each R^{c} independently is halo, —OR^{d}, —C(O)NHR^{d}, or aminoalkyl, where each R^{d} independently is H or C₁-C₅ alkyl; or
(vi) any combination of two or more of (i)-(v).

17. The method of claim 14, wherein:
when R⁶ is H and R⁹ is methyl, then R⁷ is H and R⁸ is aryl or heteroaryl; or
when R⁶ is aryl and R⁹ is H, then R⁷ is aryl and R⁸ is methyl; or
when R⁶ is alkyl and R⁹ is H, then R⁷ is H and R⁸ is methyl; or
when R⁶ is halo and R⁹ is methyl, then R⁷ and R⁸ are not H.

18. The method of claim 14, wherein:
(i) the compound has a structure according to formula I 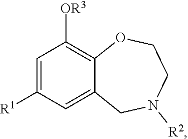
wherein
R¹ is 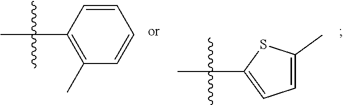
R² is 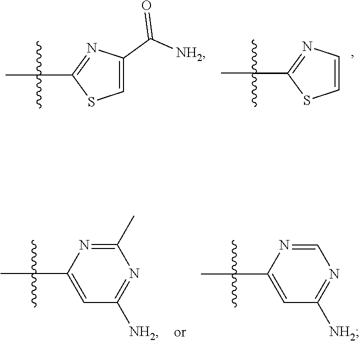
and R³ is H; or
(ii) the compound has a structure according to formula II 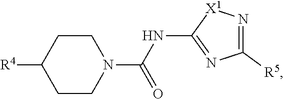
wherein
X¹ is S, R⁵ is —CH₃, R⁴ is —S—R^{a}, —CH₂—R^{a}, or F, and R^{a} is 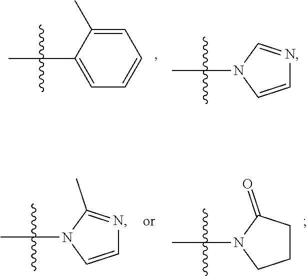
or
(iii) the compound has a structure according to formula III 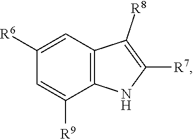
wherein
R⁶ is —CH₂N(H)C(O)(CH₂)ₘ—R^{b}, H, F, or —CH₃, where m is 0, 1, or 2, and R^{b} is 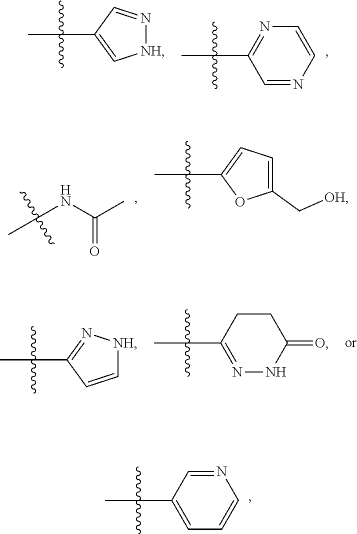
R⁷ is 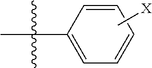
where X is halo, or R⁷ is H or —CH₂CH₃,
R⁸ is —CH₃ 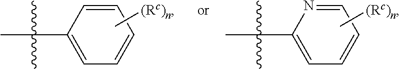
where n is 1 or 2, and each R^{c} independently is halo, —OR^{d}, —C(O)NHR^{d}, or aminoalkyl, where each R^{d} independently is H or C₁-C₃ alkyl, and
R⁹ is H, —CH₃, or —CH₂N(H)C(O)(CH₂)ₘ—R^{b}, where m is 3, and R^{b} is

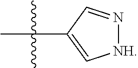 [SMILES: *c1cn[nH]c1]

19. The method of claim 14, wherein the compound has a structure according to formula IIIA, IIIB, or IIIC: 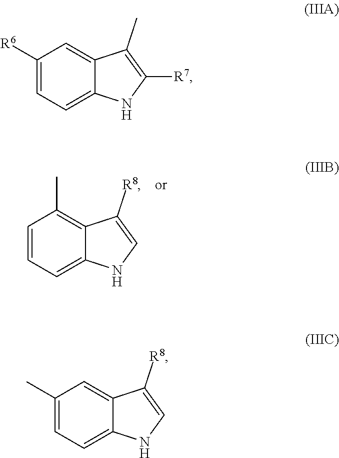
where
R⁶ is —CH₂N(H)Y(CH₂)ₘ—R^{b};
Y is CO or S(O)₂;
R^{b} is a heterocycle or —N(H)C(O)(CH₂)ₘCH₃;
R⁷ is aryl or heteroaryl; and
R⁸ is aryl or a heterocycle.

20. The method of claim 19, wherein:
(i) the compound has a structure according to formula IIIA, where R⁷ is fluorophenyl, H, or —CH₂CH₃, and R⁶ is 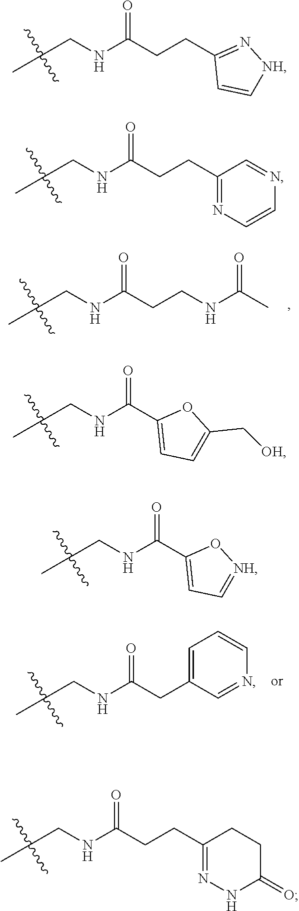
or
(ii) the compound has a structure according to formula IIIB or IIIC, where R⁸ is

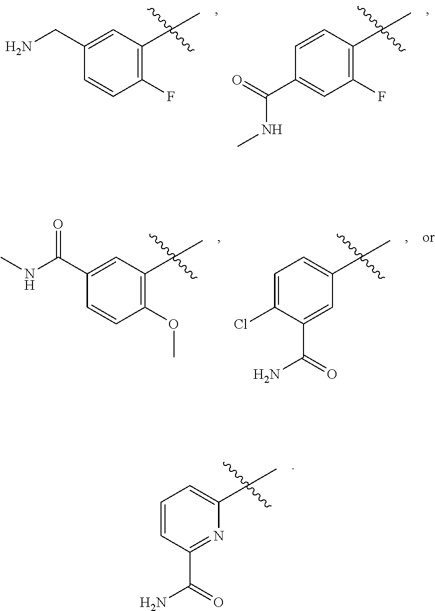

21. The method of claim 14, wherein compound is: 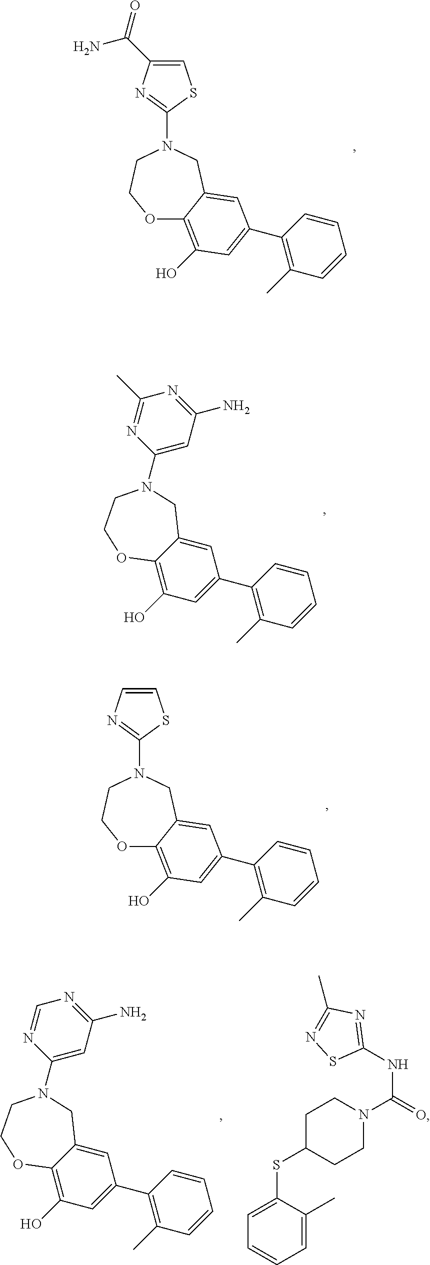 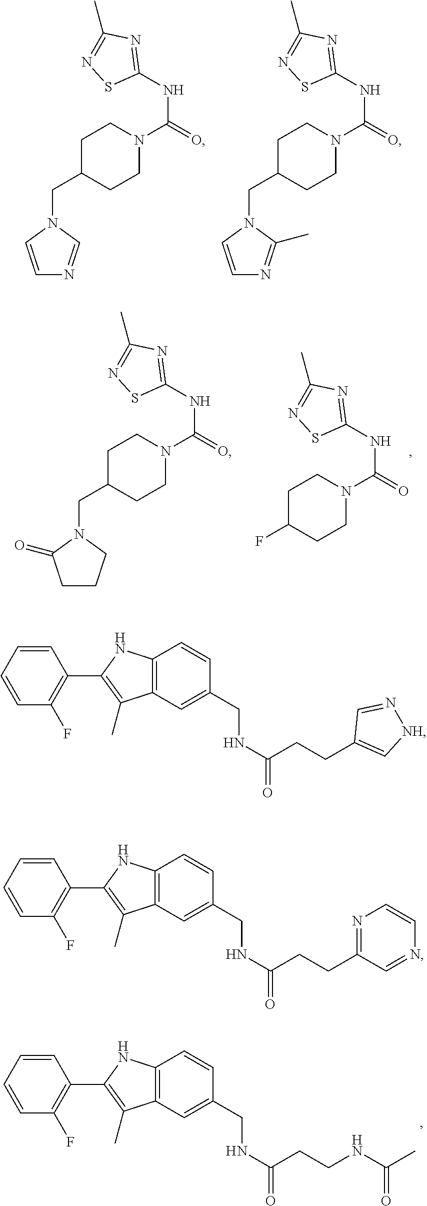 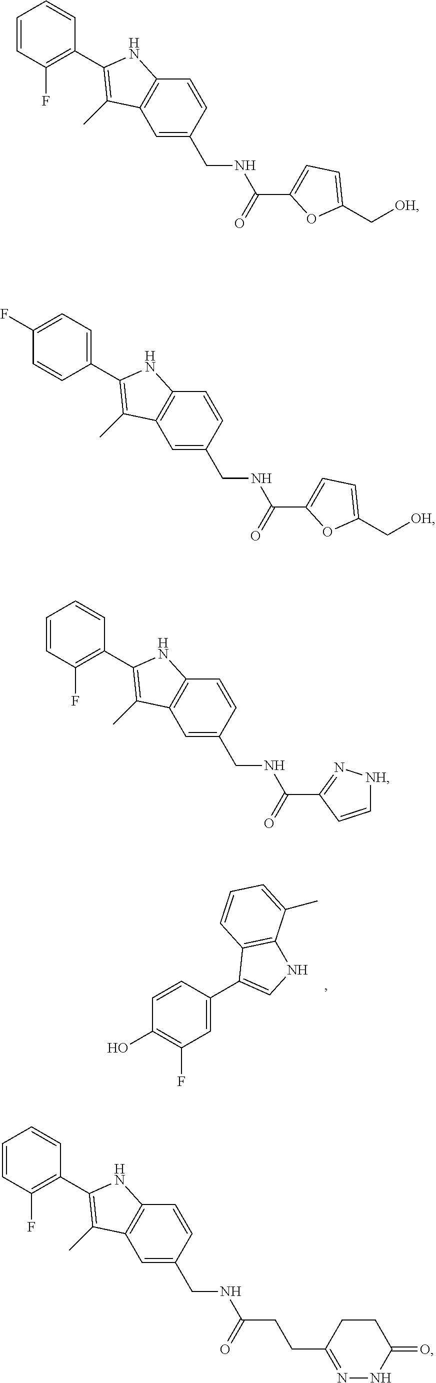 [SMILES: Cc1c(-c2ccccc2F)[nH]c2ccc(CNC(=O)c3ccc(CO)o3)cc12 ; Cc1c(-c2ccc(F)cc2)[nH]c2ccc(CNC(=O)c3ccc(CO)o3)cc12 ; Cc1c(-c2ccccc2F)[nH]c2ccc(CNC(=O)c3cc[nH]n3)cc12 ; Cc1cccc2c(-c3ccc(O)c(F)c3)c[nH]c12 ; Cc1c(-c2ccccc2F)[nH]c2ccc(CNC(=O)CCC3=NNC(=O)CC3)cc12] 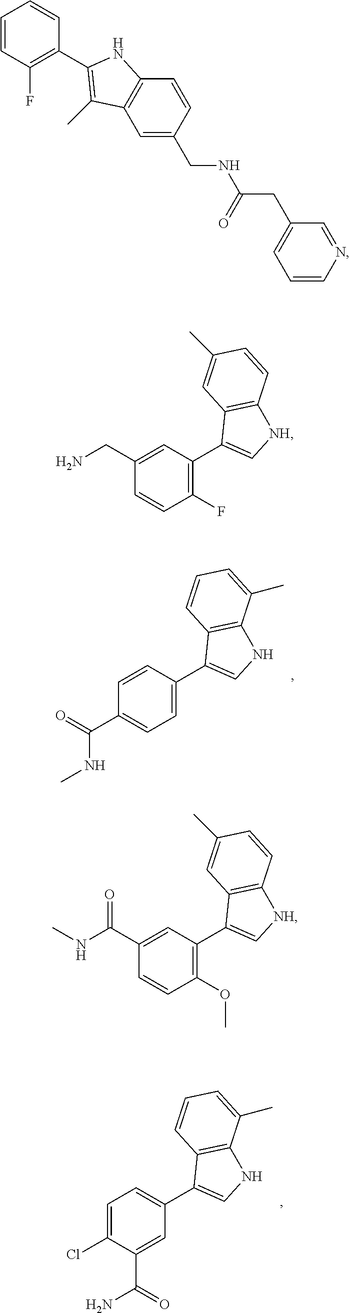 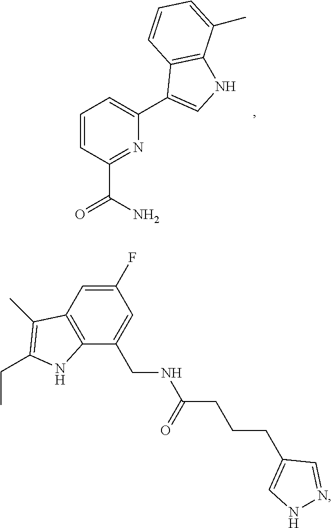
or any combination thereof.

22. The method of claim 14, wherein the compound is: 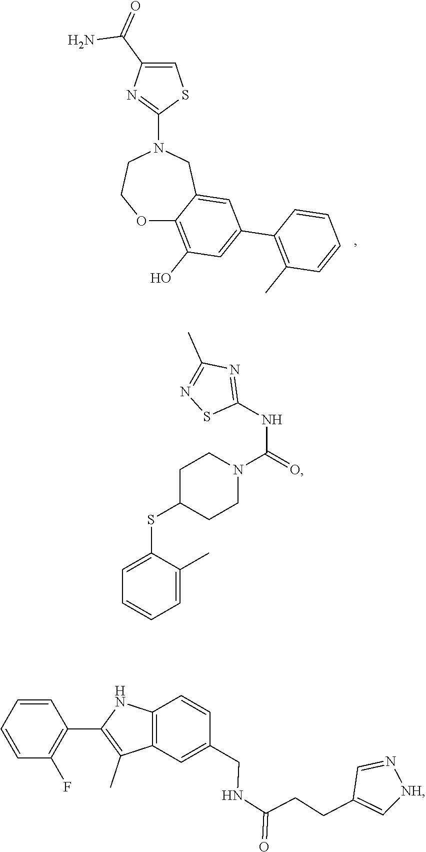 [SMILES: Cc1ccccc1-c1cc(O)c2c(c1)CN(c1nc(C(N)=O)cs1)CCO2 ; Cc1nsc(NC(=O)N2CCC(Sc3ccccc3C)CC2)n1 ; Cc1c(-c2ccccc2F)[nH]c2ccc(CNC(=O)CCc3cn[nH]c3)cc12]
or any combination thereof.

23. The method of claim 14, wherein the prostate cancer is castration-resistant prostate cancer.

24. (canceled)
